SEQUENCE LISTING (62 sequences)

SEQ ID NO: 1 - protein, 1476 aa, Streptococcus mutans
MDKKVRYKLRKVKKRWVTVSVASAVMTLTTLSGGLVKADSNESKSQISNDSNTSVVTANEESNVTTEATSKQEAASSQTNHTVTTSSSSTSVVNPKEVVSNPYTVGETASNGEKLQNQTTTVDKTSEAAANNISKQTTEADTDVIDDSNAANIQILEKLPNVKEIDGKYYYYDNNGKVRTNFTLIADGKILHFDETGAYTDTSIDTVNKDIVTTRSNLYKKYNQVYDRSAQSFEHVDHYLTAESWYRPKYILKDGKTWTQSTEKDFRPLLMTWWPSQETQRQYVNFMNAQLGINKTYDDTSNQLQLNIAAATIQAKIEAKITTLKNTDWLRQTISAFVKTQSAWNSDSEKPFDDHLQNGAVLYDNEGKLTPYANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKAMDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFNISDNKEINFLPKTLLNQDSQVGFSYDGKGYVYYSTSGYQAKNTFISEGDKWYYFDNNGYMVTGAQSINGVNYYFLPNGLQLRDAILKNEDGTYAYYGNDGRRYENGYYQFMSGVWRHFNNGEMSVGLTVIDGQVQYFDEMGYQAKGKFVTTADGKIRYFDKQSGNMYRNRFIENEEGKWLYLGEDGAAVTGSQTINGQHLYFRANGVQVKGEFVTDRHGRISYYDGNSGDQIRNRFVRNAQGQWFYFDNNGYAVTGARTINGQHLYFRANGVQVKGEFVTDRHGRISYYDGNSGDQIRNRFVRNAQGQWFYFDNNGYAVTGARTINGQHLYFRANGVQVKGEFVTDRYGRISYYDGNSGDQIRNRFVRNAQGQWFYFDNNGYAVTGARTINGQHLYFRANGVQVKGEFVTDRYGRISYYDANSGERVRIN

SEQ ID NO: 2 - DNA, 3942 bp, Streptococcus mutans
atgattgacggcaaatactactactatgacaacaacggcaaagtacgcaccaatttcacgttgatcgcggacggtaaaatcctgcattttgatgaaactggcgcgtacaccgacactagcattgataccgtgaacaaggatattgtcacgacgcgtagcaacctgtataagaaatacaatcaagtgtatgatcgcagcgcgcagagcttcgagcatgttgatcactacctgacggcggaatcttggtaccgtccgaaatacattctgaaagatggcaagacctggacccagagcaccgagaaggacttccgtcctctgctgatgacctggtggccgagccaggaaacgcagcgccagtatgtcaacttcatgaacgcccagttgggtatcaacaaaacgtacgacgacaccagcaatcagctgcaattgaacatcgctgctgcaacgatccaagcaaagatcgaagccaaaatcacgacgctgaagaacaccgattggctgcgtcaaacgatcagcgcgttcgtcaaaacccaaagcgcttggaatagcgacagcgaaaagccgtttgatgaccatctgcaaaacggtgcggttctgtatgataacgaaggtaaattgacgccgtatgccaatagcaactatcgtattctgaaccgcacgccgaccaaccagaccggtaagaaggacccgcgttataccgccgacaacacgatcggcggctacgagtttctgctggccaacgacgtggataatagcaacccggtggttcaggccgagcagctgaactggctgcacttcctgatgaactttggtaatatctacgcaaacgaccctgacgctaacttcgactccatccgcgttgacgctgtcgataatgtggacgccgatctgttacagatcgcgggtgactatctgaaagcggcaaagggcatccataagaatgacaaagcggcgaacgaccacctgtccattctggaagcgtggagcgacaatgacactccgtatctgcatgatgatggcgacaacatgattaacatggataacaaactgcgcctgagcctgctgttctccctggcgaaaccgctgaatcagcgtagcggtatgaacccgttgattacgaacagcctggtcaaccgtactgatgataatgccgaaacggcggcagtgccaagctactcttttatccgtgcccacgatagcgaggtccaggatttgattcgtgatatcattaaggctgagattaacccgaacgtcgtcggttacagcttcacgatggaagagattaagaaggcatttgagatctacaataaggacctgttggccacggagaagaagtatacccactataacaccgcattgagctacgcgttgctgctgacgaacaagagcagcgtgccgcgtgtctactatggtgatatgtttacggacgatggtcaatacatggcccacaagaccattaactacgaggcaatcgaaaccctgctgaaagcacgtatcaagtacgtgtccggtggtcaggctatgcgcaaccagcaagtgggtaattcggagatcatcaccagcgtgcgttacggtaaaggtgcgctgaaggcgatggatacgggtgaccgcactacccgtacctctggtgtggcggtcattgagggcaacaacccgagcttgcgcctgaaggcttctgatcgtgtggttgtgaatatgggtgcggcccacaaaaatcaagcctatcgcccgctgctgttgacgaccgataacggcattaaggcctatcacagcgaccaagaagcggcaggcctggtgcgttacaccaacgaccgtggcgaactgatctttaccgcagccgacattaagggctacgcaaatccgcaagttagcggctacctgggcgtctgggtccctgttggcgcagcagctgatcaggacgttcgtgttgcggcgagcaccgcgccaagcacggacggcaagagcgttcaccagaacgcggctctggacagccgtgtgatgttcgagggtttctcgaacttccaggcatttgctaccaagaaagaagagtataccaatgtggtcatcgctaagaatgtggataagttcgcggagtggggtgtcaccgatttcgagatggctccgcaatacgtttctagcaccgacggtagctttttggatagcgtgattcaaaacggttatgcttttaccgaccgttacgacctgggcatcagcaagccgaacaaatatggcaccgcggacgatctggttaaagcgattaaggcattgcacagcaaaggcatcaaagttatggcggattgggttccggaccagatgtatgccctgccggaaaaagaggttgtgacggcaacccgtgttgacaaatacggtacgccggtagctggcagccagatcaaaaacacgctgtacgtggtcgatggtaaatctagcggtaaggaccagcaggcgaagtacggtggtgccttcctggaagagctgcaagcgaagtatccggaactgttcgcgcgcaaacagattagcaccggtgttccgatggacccgagcgtcaagattaagcaatggagcgcaaaatacttcaacggcacgaatatcctgggtcgtggtgctggttacgtgctgaaagatcaggcaaccaacacctactttaacatcagcgacaataaagagatcaatttcctgccaaagacgttgctgaaccaggattctcaagttggctttagctacgacggtaagggctatgtgtactacagcacctcgggctaccaggctaaaaacacgttcatcagcgagggtgacaagtggtattacttcgacaataacggttatatggttaccggcgcacagagcattaatggtgtgaactattacttcctgccgaatggtttacagctgcgtgatgcgattctgaaaaatgaggacggtacgtacgcgtattatggcaatgatggtcgccgctacgagaatggctattatcagtttatgagcggtgtttggcgccatttcaataatggcgagatgtccgttggtctgaccgtcattgacggtcaagttcaatactttgacgagatgggttaccaggcgaaaggcaaattcgttaccaccgcggatggtaagatccgttacttcgataagcagagcggcaatatgtatcgtaatcgtttcattgagaacgaagagggcaaatggctgtacctgggtgaggacggcgcggcagtcaccggtagccagacgatcaatggtcagcacctgtattttcgtgctaacggcgttcaggttaagggtgagttcgtgaccgatcgtcatggccgcatctcttattacgacggcaacagcggtgatcagatccgcaaccgtttcgtccgcaatgcgcaaggccagtggttttactttgacaacaatggctatgcagtaactggtgctcgtacgatcaacggccagcacctgtatttccgcgcgaacggtgttcaggtaaaaggtgagtttgttacggaccgccacggccgcattagctattatgatggtaatagcggtgaccaaattcgcaatcgtttcgtgcgtaatgcacagggtcagtggttctacttcgacaataatggttatgcagtcacgggtgcacgtaccattaacggccaacacctgtactttcgcgccaatggtgtgcaagtgaaaggcgaatttgttactgatcgttatggtcgtatcagctactatgatggcaattctggcgaccaaattcgcaatcgctttgttcgtaacgcccaaggtcaatggttctatttcgacaacaacggttacgcggtgaccggtgcccgcacgattaatggtcaacacttgtacttccgtgccaacggtgtccaggtgaagggtgaatttgtgaccgaccgctatggtcgcatttcttactacgacgcaaattccggtgaacgcgtccgtatcaattaa

SEQ ID NO: 3 - protein, 1313 aa, Streptococcus mutans
MIDGKYYYYDNNGKVRTNFTLIADGKILHFDETGAYTDTSIDTVNKDIVTTRSNLYKKYNQVYDRSAQSFEHVDHYLTAESWYRPKYILKDGKTWTQSTEKDFRPLLMTWWPSQETQRQYVNFMNAQLGINKTYDDTSNQLQLNIAAATIQAKIEAKITTLKNTDWLRQTISAFVKTQSAWNSDSEKPFDDHLQNGAVLYDNEGKLTPYANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKAMDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFNISDNKEINFLPKTLLNQDSQVGFSYDGKGYVYYSTSGYQAKNTFISEGDKWYYFDNNGYMVTGAQSINGVNYYFLPNGLQLRDAILKNEDGTYAYYGNDGRRYENGYYQFMSGVWRHFNNGEMSVGLTVIDGQVQYFDEMGYQAKGKFVTTADGKIRYFDKQSGNMYRNRFIENEEGKWLYLGEDGAAVTGSQTINGQHLYFRANGVQVKGEFVTDRHGRISYYDGNSGDQIRNRFVRNAQGQWFYFDNNGYAVTGARTINGQHLYFRANGVQVKGEFVTDRHGRISYYDGNSGDQIRNRFVRNAQGQWFYFDNNGYAVTGARTINGQHLYFRANGVQVKGEFVTDRYGRISYYDGNSGDQIRNRFVRNAQGQWFYFDNNGYAVTGARTINGQHLYFRANGVQVKGEFVTDRYGRISYYDANSGERVRIN

SEQ ID NO: 4 - protein, 1146 aa, Paenibacillus humicus
MRIRTKYMNWMLVLVLIAAGFFQAAGPIAPATAAGGANLTLGKTVTASGQSQTYSPDNVKDSNQGTYWESTNNAFPQWIQVDLGASTSIDQIVLKLPSGWETRTQTLSIQGSANGSTFTNIVGSAGYTFNPSVAGNSVTINFSAASARYVRLNFTANTGWPAGQLSELEIYGATAPTPTPTPTPTPTPTPTPTPTPTVTPAPSATPTPTPPAGSNIAVGKSITASSSTQTYVAANANDNNTSTYWEGGSNPSTLTLDFGSNQSITSVVLKLNPASEWGTRTQTIQVLGADQNAGSFSNLVSAQSYTFNPATGNTVTIPVSATVKRLQLNITANSGAPAGQIAEFQVFGTPAPNPDLTITGMSWTPSSPVESGDITLNAVVKNIGTAAAGATTVNFYLNNELAGTAPVGALAAGASANVSINAGAKAAATYAVSAKVDESNAVIEQNEGNNSYSNPTNLVVAPVSSSDLVAVTSWSPGTPSQGAAVAFTVALKNQGTLASAGGAHPVTVVLKNAAGATLQTFTGTYTGSLAAGASANISVGSWTAASGTYTVSTTVAADGNEIPAKQSNNTSSASLTVYSARGASMPYSRYDTEDAVLGGGAVLRTAPTFDQSLIASEASGQKYAALPSNGSSLQWTVRQGQGGAGVTMRFTMPDTSDGMGQNGSLDVYVNGTKAKTVSLTSYYSWQYFSGDMPADAPGGGRPLFRFDEVHFKLDTALKPGDTIRVQKGGDSLEYGVDFIEIEPIPAAVARPANSVSVTEYGAVANDGKDDLAAFKAAVTAAVAAGKSLYIPEGTFHLSSMWEIGSATSMIDNFTVTGAGIWYTNIQFTNPNASGGGISLRIKGKLDFSNIYMNSNLRSRYGQNAVYKGFMDNFGTNSIIHDVWVEHFECGMWVGDYAHTPAIYASGLVVENSRIRNNLADGINFSQGTSNSTVRNSSIRNNGDDGLAVWTSNTNGAPAGVNNTFSYNTIENNWRAAAIAFFGGSGHKADHNYIIDCVGGSGIRMNTVFPGYHFQNNTGITFSDTTIINSGTSQDLYNGERGAIDLEASNDAIKNVTFTNIDIINAQRDGVQIGYGGGFENIVFNNITIDGTGRDGISTSRFSGPHLGAAIYTYTGNGSATFNNLVTRNIAYAGGNYIQSGFNLTIK

SEQ ID NO: 5 - DNA, 3351 bp, Paenibacillus humicus
atggctagcgcagcaggaggcgcgaatctgacgctcggcaaaaccgtcaccgccagcggccagtcgcagacgtacagccccgacaatgtcaaggacagcaatcagggaacttactgggaaagcacgaacaacgccttcccgcagtggatccaagtcgaccttggcgccagcacgagcatcgaccagatcgtgctcaaacttccgtccggatgggagactcgtacgcaaacgctctcgatacagggcagcgcgaacggctcgacgttcacgaacatcgtcggatcggccgggtatacattcaatccatccgtcgccggcaacagcgtcacgatcaacttcagcgctgccagcgcccgctacgtccgcctgaatttcacggccaatacgggctggccagcaggccagctgtcggagcttgagatctacggagcgacggcgccaacgcctactcccacgcctactccaacaccaacgccaacgccaacaccaacgccaacccctacagtaacccctgcgccttcggccacgccgactccgactcctccggcaggcagcaacatcgccgtagggaaatcgattacagcctcttccagcacgcagacctacgtagctgcaaatgcaaatgacaacaatacatccacctattgggagggaggaagcaacccgagcacgctgactctcgatttcggttccaaccagagcatcacttccgtcgtcctcaagctgaatccggcttcggaatgggggactcgcacgcaaacgatccaagttcttggagcggatcagaacgccggctccttcagcaatctcgtctctgcccagtcctatacgttcaatcccgcaaccggcaatacggtgacgattccggtctccgcgacggtcaagcgcctccagctgaacattacggcgaactccggcgcccctgccggccagattgccgagttccaagtgttcggcacgccagcgcctaatccggacttgaccattaccggcatgtcctggactccgtcttctccggtcgagagcggcgacattacgctgaacgccgtcgtcaagaacatcggaactgcagctgcaggcgccacgacggtcaatttctacctgaacaacgaactcgccggcaccgctccggtaggcgcgcttgcggcaggagcttctgcaaatgtatcgatcaatgcaggcgccaaagcagccgcaacgtatgcggtaagcgccaaagtcgacgagagcaacgccgtcatcgagcagaatgaaggcaacaacagctactcgaacccgactaacctcgtcgtagcgccggtgtccagctccgacctcgtcgccgtgacgtcatggtcgccgggcacgccgtcgcagggagcggcggtcgcatttaccgtcgcgcttaaaaatcagggtacgctggcttccgccggcggagcccatcccgtaaccgtcgttctgaaaaacgctgccggagcgacgctgcaaaccttcacgggcacctacacaggttccctggcagcaggcgcatccgcgaatatcagcgtgggcagctggacggcagcgagcggcacctataccgtctcgacgacggtagccgctgacggcaatgaaattccggccaagcaaagcaacaatacgagcagcgcgagcctcacggtctactcggcgcgcggcgccagcatgccgtacagccgttacgacacggaggatgcggtgctcggcggcggagctgtcctgagaacggcgccgacgttcgatcagtcgctcatcgcttccgaagcatcgggacagaaatacgccgcacttccgtccaacggctccagcctgcagtggaccgtccgtcaaggccagggcggtgcaggcgtcacgatgcgcttcacgatgcccgacacgagcgacggcatgggccagaacggctcgctcgacgtctatgtcaacggaaccaaagccaaaacggtgtcgctgacctcttattacagctggcagtatttctccggcgacatgccggctgacgctccgggcggcggcaggccgctcttccgcttcgacgaagtccacttcaagctggatacggcgttgaagccgggagacacgatccgcgtccagaagggcggtgacagcctggagtacggcgtcgacttcatcgagatcgagccgattccggcagcggttgcccgtccggccaactcggtgtccgtcaccgaatacggcgctgtcgccaatgacggcaaggatgatctcgccgccttcaaggctgccgtgaccgcagcggtagcggccggaaaatccctctacatcccggaaggcaccttccacctgagcagcatgtgggagatcggctcggccaccagcatgatcgacaacttcacggtcacgggtgccggcatctggtatacgaacatccagttcacgaatcccaatgcatcgggcggcggcatctccctgagaatcaaaggaaagctggatttcagcaacatctacatgaactccaacctgcgttcccgttacgggcagaacgccgtctacaaaggctttatggacaatttcggcactaattcgatcatccatgacgtctgggtcgagcatttcgaatgcggcatgtgggtcggcgactacgcccatactcctgcgatctatgcgagcgggctcgtcgtggaaaacagccgcatccgcaacaatcttgccgacggcatcaacttctcgcagggaacgagcaactcgaccgtccgcaacagcagcatccgcaacaacggcgatgacggcctcgccgtctggacgagcaacacgaacggcgctccggccggcgtgaacaacaccttctcctacaacacgatcgagaacaactggcgcgcggcggccatcgccttcttcggcggcagcggccacaaggctgaccacaactacatcatcgactgtgtcggcggctccggcatccggatgaatacggtgttcccaggctaccacttccagaacaacaccggcatcaccttctcggatacgacgatcatcaacagcggcaccagccaggatctgtacaacggcgagcgcggagcgattgatctggaagcatccaacgacgcgatcaaaaacgtcaccttcaccaacatcgacatcatcaatgcccagcgcgacggcgttcagatcggctatggcggcggcttcgagaacatcgtgttcaacaacatcacgatcgacggcaccggccgcgacgggatatcgacatcccgcttctcgggacctcatcttggcgcagccatctatacgtacacgggcaacggctcggcgacgttcaacaacctggtgacccggaacatcgcctatgcaggcggcaactacatccagagcgggttcaacctgacgatctaa

SEQ ID NO: 6 - protein, 1116 aa, Paenibacillus humicus
MASAAGGANLTLGKTVTASGQSQTYSPDNVKDSNQGTYWESTNNAFPQWIQVDLGASTSIDQIVLKLPSGWETRTQTLSIQGSANGSTFTNIVGSAGYTFNPSVAGNSVTINFSAASARYVRLNFTANTGWPAGQLSELEIYGATAPTPTPTPTPTPTPTPTPTPTPTVTPAPSATPTPTPPAGSNIAVGKSITASSSTQTYVAANANDNNTSTYWEGGSNPSTLTLDFGSNQSITSVVLKLNPASEWGTRTQTIQVLGADQNAGSFSNLVSAQSYTFNPATGNTVTIPVSATVKRLQLNITANSGAPAGQIAEFQVFGTPAPNPDLTITGMSWTPSSPVESGDITLNAVVKNIGTAAAGATTVNFYLNNELAGTAPVGALAAGASANVSINAGAKAAATYAVSAKVDESNAVIEQNEGNNSYSNPTNLVVAPVSSSDLVAVTSWSPGTPSQGAAVAFTVALKNQGTLASAGGAHPVTVVLKNAAGATLQTFTGTYTGSLAAGASANISVGSWTAASGTYTVSTTVAADGNEIPAKQSNNTSSASLTVYSARGASMPYSRYDTEDAVLGGGAVLRTAPTFDQSLIASEASGQKYAALPSNGSSLQWTVRQGQGGAGVTMRFTMPDTSDGMGQNGSLDVYVNGTKAKTVSLTSYYSWQYFSGDMPADAPGGGRPLFRFDEVHFKLDTALKPGDTIRVQKGGDSLEYGVDFIEIEPIPAAVARPANSVSVTEYGAVANDGKDDLAAFKAAVTAAVAAGKSLYIPEGTFHLSSMWEIGSATSMIDNFTVTGAGIWYTNIQFTNPNASGGGISLRIKGKLDFSNIYMNSNLRSRYGQNAVYKGFMDNFGTNSIIHDVWVEHFECGMWVGDYAHTPAIYASGLVVENSRIRNNLADGINFSQGTSNSTVRNSSIRNNGDDGLAVWTSNTNGAPAGVNNTFSYNTIENNWRAAAIAFFGGSGHKADHNYIIDCVGGSGIRMNTVFPGYHFQNNTGITFSDTTIINSGTSQDLYNGERGAIDLEASNDAIKNVTFTNIDIINAQRDGVQIGYGGGFENIVFNNITIDGTGRDGISTSRFSGPHLGAAIYTYTGNGSATFNNLVTRNIAYAGGNYIQSGFNLTI

SEQ ID NO: 7 - protein, 26 aa, Bacillus subtilis
MRSKKLWISLLFALTLIFTMAFSNMS

SEQ ID NO: 8 - DNA, 3426 bp, Paenibacillus humicus
gtgagaagcaaaaaattgtggatcagcttgttgtttgcgttaacgttaatctttacgatggcgttcagcaacatgtctgctagcgcagcaggaggcgcgaatctgacgctcggcaaaaccgtcaccgccagcggccagtcgcagacgtacagccccgacaatgtcaaggacagcaatcagggaacttactgggaaagcacgaacaacgccttcccgcagtggatccaagtcgaccttggcgccagcacgagcatcgaccagatcgtgctcaaacttccgtccggatgggagactcgtacgcaaacgctctcgatacagggcagcgcgaacggctcgacgttcacgaacatcgtcggatcggccgggtatacattcaatccatccgtcgccggcaacagcgtcacgatcaacttcagcgctgccagcgcccgctacgtccgcctgaatttcacggccaatacgggctggccagcaggccagctgtcggagcttgagatctacggagcgacggcgccaacgcctactcccacgcctactccaacaccaacgccaacgccaacaccaacgccaacccctacagtaacccctgcgccttcggccacgccgactccgactcctccggcaggcagcaacatcgccgtagggaaatcgattacagcctcttccagcacgcagacctacgtagctgcaaatgcaaatgacaacaatacatccacctattgggagggaggaagcaacccgagcacgctgactctcgatttcggttccaaccagagcatcacttccgtcgtcctcaagctgaatccggcttcggaatgggggactcgcacgcaaacgatccaagttcttggagcggatcagaacgccggctccttcagcaatctcgtctctgcccagtcctatacgttcaatcccgcaaccggcaatacggtgacgattccggtctccgcgacggtcaagcgcctccagctgaacattacggcgaactccggcgcccctgccggccagattgccgagttccaagtgttcggcacgccagcgcctaatccggacttgaccattaccggcatgtcctggactccgtcttctccggtcgagagcggcgacattacgctgaacgccgtcgtcaagaacatcggaactgcagctgcaggcgccacgacggtcaatttctacctgaacaacgaactcgccggcaccgctccggtaggcgcgcttgcggcaggagcttctgcaaatgtatcgatcaatgcaggcgccaaagcagccgcaacgtatgcggtaagcgccaaagtcgacgagagcaacgccgtcatcgagcagaatgaaggcaacaacagctactcgaacccgactaacctcgtcgtagcgccggtgtccagctccgacctcgtcgccgtgacgtcatggtcgccgggcacgccgtcgcagggagcggcggtcgcatttaccgtcgcgcttaaaaatcagggtacgctggcttccgccggcggagcccatcccgtaaccgtcgttctgaaaaacgctgccggagcgacgctgcaaaccttcacgggcacctacacaggttccctggcagcaggcgcatccgcgaatatcagcgtgggcagctggacggcagcgagcggcacctataccgtctcgacgacggtagccgctgacggcaatgaaattccggccaagcaaagcaacaatacgagcagcgcgagcctcacggtctactcggcgcgcggcgccagcatgccgtacagccgttacgacacggaggatgcggtgctcggcggcggagctgtcctgagaacggcgccgacgttcgatcagtcgctcatcgcttccgaagcatcgggacagaaatacgccgcacttccgtccaacggctccagcctgcagtggaccgtccgtcaaggccagggcggtgcaggcgtcacgatgcgcttcacgatgcccgacacgagcgacggcatgggccagaacggctcgctcgacgtctatgtcaacggaaccaaagccaaaacggtgtcgctgacctcttattacagctggcagtatttctccggcgacatgccggctgacgctccgggcggcggcaggccgctcttccgcttcgacgaagtccacttcaagctggatacggcgttgaagccgggagacacgatccgcgtccagaagggcggtgacagcctggagtacggcgtcgacttcatcgagatcgagccgattccggcagcggttgcccgtccggccaactcggtgtccgtcaccgaatacggcgctgtcgccaatgacggcaaggatgatctcgccgccttcaaggctgccgtgaccgcagcggtagcggccggaaaatccctctacatcccggaaggcaccttccacctgagcagcatgtgggagatcggctcggccaccagcatgatcgacaacttcacggtcacgggtgccggcatctggtatacgaacatccagttcacgaatcccaatgcatcgggcggcggcatctccctgagaatcaaaggaaagctggatttcagcaacatctacatgaactccaacctgcgttcccgttacgggcagaacgccgtctacaaaggctttatggacaatttcggcactaattcgatcatccatgacgtctgggtcgagcatttcgaatgcggcatgtgggtcggcgactacgcccatactcctgcgatctatgcgagcgggctcgtcgtggaaaacagccgcatccgcaacaatcttgccgacggcatcaacttctcgcagggaacgagcaactcgaccgtccgcaacagcagcatccgcaacaacggcgatgacggcctcgccgtctggacgagcaacacgaacggcgctccggccggcgtgaacaacaccttctcctacaacacgatcgagaacaactggcgcgcggcggccatcgccttcttcggcggcagcggccacaaggctgaccacaactacatcatcgactgtgtcggcggctccggcatccggatgaatacggtgttcccaggctaccacttccagaacaacaccggcatcaccttctcggatacgacgatcatcaacagcggcaccagccaggatctgtacaacggcgagcgcggagcgattgatctggaagcatccaacgacgcgatcaaaaacgtcaccttcaccaacatcgacatcatcaatgcccagcgcgacggcgttcagatcggctatggcggcggcttcgagaacatcgtgttcaacaacatcacgatcgacggcaccggccgcgacgggatatcgacatcccgcttctcgggacctcatcttggcgcagccatctatacgtacacgggcaacggctcggcgacgttcaacaacctggtgacccggaacatcgcctatgcaggcggcaactacatccagagcgggttcaacctgacgatctaa

SEQ ID NO: 9 - protein, 1141 aa, Paenibacillus humicus
MRSKKLWISLLFALTLIFTMAFSNMSASAAGGANLTLGKTVTASGQSQTYSPDNVKDSNQGTYWESTNNAFPQWIQVDLGASTSIDQIVLKLPSGWETRTQTLSIQGSANGSTFTNIVGSAGYTFNPSVAGNSVTINFSAASARYVRLNFTANTGWPAGQLSELEIYGATAPTPTPTPTPTPTPTPTPTPTPTVTPAPSATPTPTPPAGSNIAVGKSITASSSTQTYVAANANDNNTSTYWEGGSNPSTLTLDFGSNQSITSVVLKLNPASEWGTRTQTIQVLGADQNAGSFSNLVSAQSYTFNPATGNTVTIPVSATVKRLQLNITANSGAPAGQIAEFQVFGTPAPNPDLTITGMSWTPSSPVESGDITLNAVVKNIGTAAAGATTVNFYLNNELAGTAPVGALAAGASANVSINAGAKAAATYAVSAKVDESNAVIEQNEGNNSYSNPTNLVVAPVSSSDLVAVTSWSPGTPSQGAAVAFTVALKNQGTLASAGGAHPVTVVLKNAAGATLQTFTGTYTGSLAAGASANISVGSWTAASGTYTVSTTVAADGNEIPAKQSNNTSSASLTVYSARGASMPYSRYDTEDAVLGGGAVLRTAPTFDQSLIASEASGQKYAALPSNGSSLQWTVRQGQGGAGVTMRFTMPDTSDGMGQNGSLDVYVNGTKAKTVSLTSYYSWQYFSGDMPADAPGGGRPLFRFDEVHFKLDTALKPGDTIRVQKGGDSLEYGVDFIEIEPIPAAVARPANSVSVTEYGAVANDGKDDLAAFKAAVTAAVAAGKSLYIPEGTFHLSSMWEIGSATSMIDNFTVTGAGIWYTNIQFTNPNASGGGISLRIKGKLDFSNIYMNSNLRSRYGQNAVYKGFMDNFGTNSIIHDVWVEHFECGMWVGDYAHTPAIYASGLVVENSRIRNNLADGINFSQGTSNSTVRNSSIRNNGDDGLAVWTSNTNGAPAGVNNTFSYNTIENNWRAAAIAFFGGSGHKADHNYIIDCVGGSGIRMNTVFPGYHFQNNTGITFSDTTIINSGTSQDLYNGERGAIDLEASNDAIKNVTFTNIDIINAQRDGVQIGYGGGFENIVFNNITIDGTGRDGISTSRFSGPHLGAAIYTYTGNGSATFNNLVTRNIAYAGGNYIQSGFNLTI

SEQ ID NO: 10 - DNA, 1308 bp, Penicillium marneffei
atgaagcaaaccacttccctcctcctctcagccatcgcggcaaccagcagcttcagcggactaacagccgctcaaaaactcgcctttgcgcacgtcgtcgtcggcaacactgcagcacacacccaatccacctgggaaagcgacattactctcgcccataactccggtctagatgcctttgccttgaacggtggattccccgatggcaacatccccgcacaaatcgccaacgcttttgcggcttgtgaagccctttcaaatggcttcaagctattcatttcgtttgactacctcggtggtggtcagccctggcctgcctcagaggttgtgtctatgctgaagcagtatgccagttccgattgttatttggcctatgatggcaagccctttgtctcaacttttgagggcaccggaaatattgcggattgggcgcacggaggtcccattcggtcggcggtggatgtttactttgtgccggattggacgagtttggggcctgctgggattaagtcgtatctcgacaatatcgatggatttttcagctggaacatgtggcctgtaggtgcggccgatatgaccgacgagcctgatttcgaatggctcgatgcaattgggtccgacaagacgtacatgatgggcgtttcgccatggttcttccacagtgcaagcggaggcaccgactgggtctggcgtggtgatgacctctgggatgaccgatggattcaagtcacctgcgtcgaccctcaatttgtccaggtcgtcacatggaacgactggggtgaatcctcctacatcggccccttcgtgaccgctagcgaagtccccgccggctcattagcctacgtcgacaacatgtcacaccaaagcttccttgacttcttgcctttctacatcgccaccttcaaaggcgacacattcaacatctcccgcgaccagatgcaatactggtaccgcctcgcacccgccgcagcaggcagcgcgtgcggcgtatacggcaatgatcccgatcaaggccagactaccgttgacgtcaactccatcgttcaggacaaggtgtttttcagtgctttgttgacggctgatgctactgtaacggtgcagattggtagtaatgctgcggtttcatatgatggtgttgctggtatgaaccactggagtcaggactttaatggccagaccggcgcggttacgtttagtgttgtcaggggtggcgctacagttaagagtggtattggagccgagattacggcttcgacttcgttgtcgaatgggtgcactaattacaacccttgggttggtagtttctaa

SEQ ID NO: 11 - protein, 435 aa, Penicillium marneffei
MKQTTSLLLSAIAATSSFSGLTAAQKLAFAHVVVGNTAAHTQSTWESDITLAHNSGLDAFALNGGFPDGNIPAQIANAFAACEALSNGFKLFISFDYLGGGQPWPASEVVSMLKQYASSDCYLAYDGKPFVSTFEGTGNIADWAHGGPIRSAVDVYFVPDWTSLGPAGIKSYLDNIDGFFSWNMWPVGAADMTDEPDFEWLDAIGSDKTYMMGVSPWFFHSASGGTDWVWRGDDLWDDRWIQVTCVDPQFVQVVTWNDWGESSYIGPFVTASEVPAGSLAYVDNMSHQSFLDFLPFYIATFKGDTFNISRDQMQYWYRLAPAAAGSACGVYGNDPDQGQTTVDVNSIVQDKVFFSALLTADATVTVQIGSNAAVSYDGVAGMNHWSQDFNGQTGAVTFSVVRGGATVKSGIGAEITASTSLSNGCTNYNPWVGSF

SEQ ID NO: 12 - DNA, 8616 bp, artificial sequence
  plasmid pTrex
aagcttaactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatcatgtatcggaagttggccgtcatctcggccttcttggccacacctcgtgctagactaggcgcgccgcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgc

SEQ ID NO: 13 - protein, 1455 aa, Streptococcus mutans
MEKKVRFKLRKVKKRWVTVSVASAVVTLTSLSGSLVKADSTDDRQQAVTESQASLVTTSEAAKETLTATDTSTATSATSQLTATVTDNVSTTNQSTNTTANTANFDVKPTTTSEQSKTDNSDKIIATSKAVNRLTATGKFVPANNNTAHPKTVTDKIVPIKPKIGKLKQPSSLSQDDIAALGNVKNIRKVNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNTFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGVYRYASHDQRNHWNYDYRRDFGRGSSSAIRFRHSRNGFFDNFFRF

SEQ ID NO: 14 - DNA, 3804 bp, Streptococcus mutans
atggtcaatggcaaatactactactacaaagaggacggtacgttgcagaagaactacgcactgaacattaacggcaagacctttttctttgacgagactggcgccctgagcaataacaccctgccgagcaagaaaggtaacatcaccaataacgacaataccaatagcttcgcgcaatacaatcaggtgtattcgacggatgcagcgaacttcgaacatgtcgatcactacctgacggcggagtcctggtatcgcccgaagtatattctgaaagatggcaagacgtggactcagtccacggagaaagattttcgcccgttgttgatgacctggtggccggatcaggaaacccagcgtcagtatgtaaactatatgaatgcccagctgggtattcaccagacctacaacacggcgaccagcccgttgcaactgaatctggcggcacagacgatccagaccaagattgaagagaagatcacggcggagaagaacactaattggctgcgtcaaacgatttcggcctttgtcaaaacccagagcgcgtggaactcggacagcgaaaaaccgtttgacgatcatctgcaaaagggtgcactgctgtactctaacaatagcaagttgacctctcaagctaatagcaactaccgtattctgaaccgtaccccaaccaaccaaaccggcaagaaagatccgcgttataccgctgaccgtaccatcggtggttatgagttcttgctggcgaacgatgtggataatagcaatcctgttgttcaagcggaacagctgaactggctgcacttcctgatgaactttggcaatatctatgcaaacgaccctgacgccaactttgacagcatccgtgtagacgccgtggacaacgtggatgcagatttgttgcaaatcgctggtgactatctgaaggctgcaaagggcatccataagaacgacaaagcagcgaacgaccacctgtcgatcctggaagcatggagctataatgacaccccgtatctgcacgacgacggtgacaacatgatcaatatggacaaccgtctgcgtctgagcctgctgtatagcctggcgaagccgttgaaccagcgttcgggcatgaacccgctgatcacgaacagcctggttaaccgtaccgatgacaacgcagaaaccgcagcggtcccgagctacagctttatccgtgcacacgatagcgaggttcaagacctgattcgtaacattattcgtgctgagattaatccgaacgtcgtcggttatagcttcacgatggaagagatcaagaaggcctttgagatttacaacaaggatctgctggcgacggaaaagaaatacacccactataacaccgcgctgagctacgcgctgctgctgaccaataagagcagcgttccgcgtgtgtattacggtgatatgtttactgacgacggtcagtacatggcacataaaacgatcaactacgaggctatcgaaacgctgttgaaggcgcgcattaagtacgtgtctggtggccaagcgatgcgtaatcaacaggtgggtaatagcgaaatcattacgagcgtccgctatggcaagggcgcactgaaagcgacggataccggcgatcgtaccacgcgcaccagcggcgttgcggttattgaaggcaataacccgagcctgcgcttgaaggcgagcgaccgcgtcgttgttaacatgggtgcagcacacaagaaccaggcatatcgtccgctgttgctgaccactgataatggcatcaaagcgtatcacagcgatcaggaagctgcgggcctggtgcgctataccaatgatcgtggtgaattgatcttcacggcagctgacattaaaggttatgcaaatccgcaagtcagcggttatctgggcgtctgggtgccggtcggcgcagcggctgatcaagacgtgcgtgtggccgcgagcaccgcgccatcgaccgacggtaaaagcgtgcaccagaatgcggcgctggacagccgtgtcatgtttgagggttttagcaactttcaagcctttgcaacgaagaaagaagagtacaccaacgtcgtcatcgcgaagaacgtcgataagttcgcggaatggggcgttaccgatttcgaaatggcaccgcagtatgtgtctagcaccgatggctcgtttctggattccgtgatccaaaatggttatgcatttaccgaccgctatgacctgggcattagcaagccgaataagtatggtacggcggatgatctggttaaagcgatcaaggcgctgcattctaaaggtattaaggttatggccgactgggttccagatcagatgtatgctttcccggaaaaagaagtggtgacggccacccgcgtggacaaatatggtacgccggtcgcgggcagccagatcaaaaacactctgtatgtcgtggatggcaaaagctccggtaaagatcagcaagcgaaatatggcggtgccttcctggaagagttgcaggcgaaatacccggaactgttcgcgcgtaagcagatcagcactggtgttccgatggacccgagcgtgaagattaaacaatggtccgcgaaatactttaacggcacgaacatcctgggtcgtggtgccggctacgtgctgaaagaccaggcaacgaatacgtactttagcttggtgtccgacaatacgtttctgccgaagtctctggtcaacccgaaccacggtacgagcagctctgtgaccggcctggtgttcgatggtaagggctacgtgtactactctaccagcggttaccaggccaagaatacgttcatcagcctgggtaacaactggtattacttcgacaataacggttacatggtcacgggtgcgcagagcatcaacggtgccaactactattttctgagcaacggcattcagctgcgtaatgcgatttacgacaatggcaataaggttctgagctactacggtaatgacggtcgtcgttatgagaatggctattacctgtttggccaacagtggcgctactttcaaaatggtattatggccgtcggtctgacccgtgtccacggtgcggtgcagtattttgacgccagcggcttccaagccaagggccagttcatcaccactgcggacggtaaactgcgttactttgaccgtgacagcggcaaccaaatcagcaatcgttttgttcgtaacagcaagggtgaatggtttttgttcgatcataacggcgtggcggttaccggcaccgttactttcaatggtcaacgtctgtactttaagccgaacggtgttcaggcaaagggtgagttcattcgcgacgcggatggtcacttgcgttactacgaccctaattccggtaatgaggttcgtaaccgtttcgtccgcaactctaagggcgaatggttcctgtttgaccacaatggcatcgcagtcaccggcgctcgtgtggtcaacggccaacgcttgtacttcaaaagcaatggcgtccaagctaagggtgagctgattaccgaacgtaagggccgtattaagtattatgatcctaacagcggtaacgaagtgcgtaaccgctacgtccgcaccagcagcggtaattggtactattttggtaacgatggttacgcgctgatcggctggcatgttgttgagggtcgtcgtgtgtactttgatgagaacggtgtctatcgttacgcgagccacgaccagcgtaatcattggaactacgactatcgtcgcgatttcggtcgtggtagcagctccgctatccgttttcgccatagccgtaacggctttttcgacaacttcttccgcttctaa

SEQ ID NO: 15 - DNA, 7790 bp, artificial sequence
  synthetic construct
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttaggaggtaaaacatatggtcaatggcaaatactactactacaaagaggacggtacgttgcagaagaactacgcactgaacattaacggcaagacctttttctttgacgagactggcgccctgagcaataacaccctgccgagcaagaaaggtaacatcaccaataacgacaataccaatagcttcgcgcaatacaatcaggtgtattcgacggatgcagcgaacttcgaacatgtcgatcactacctgacggcggagtcctggtatcgcccgaagtatattctgaaagatggcaagacgtggactcagtccacggagaaagattttcgcccgttgttgatgacctggtggccggatcaggaaacccagcgtcagtatgtaaactatatgaatgcccagctgggtattcaccagacctacaacacggcgaccagcccgttgcaactgaatctggcggcacagacgatccagaccaagattgaagagaagatcacggcggagaagaacactaattggctgcgtcaaacgatttcggcctttgtcaaaacccagagcgcgtggaactcggacagcgaaaaaccgtttgacgatcatctgcaaaagggtgcactgctgtactctaacaatagcaagttgacctctcaagctaatagcaactaccgtattctgaaccgtaccccaaccaaccaaaccggcaagaaagatccgcgttataccgctgaccgtaccatcggtggttatgagttcttgctggcgaacgatgtggataatagcaatcctgttgttcaagcggaacagctgaactggctgcacttcctgatgaactttggcaatatctatgcaaacgaccctgacgccaactttgacagcatccgtgtagacgccgtggacaacgtggatgcagatttgttgcaaatcgctggtgactatctgaaggctgcaaagggcatccataagaacgacaaagcagcgaacgaccacctgtcgatcctggaagcatggagctataatgacaccccgtatctgcacgacgacggtgacaacatgatcaatatggacaaccgtctgcgtctgagcctgctgtatagcctggcgaagccgttgaaccagcgttcgggcatgaacccgctgatcacgaacagcctggttaaccgtaccgatgacaacgcagaaaccgcagcggtcccgagctacagctttatccgtgcacacgatagcgaggttcaagacctgattcgtaacattattcgtgctgagattaatccgaacgtcgtcggttatagcttcacgatggaagagatcaagaaggcctttgagatttacaacaaggatctgctggcgacggaaaagaaatacacccactataacaccgcgctgagctacgcgctgctgctgaccaataagagcagcgttccgcgtgtgtattacggtgatatgtttactgacgacggtcagtacatggcacataaaacgatcaactacgaggctatcgaaacgctgttgaaggcgcgcattaagtacgtgtctggtggccaagcgatgcgtaatcaacaggtgggtaatagcgaaatcattacgagcgtccgctatggcaagggcgcactgaaagcgacggataccggcgatcgtaccacgcgcaccagcggcgttgcggttattgaaggcaataacccgagcctgcgcttgaaggcgagcgaccgcgtcgttgttaacatgggtgcagcacacaagaaccaggcatatcgtccgctgttgctgaccactgataatggcatcaaagcgtatcacagcgatcaggaagctgcgggcctggtgcgctataccaatgatcgtggtgaattgatcttcacggcagctgacattaaaggttatgcaaatccgcaagtcagcggttatctgggcgtctgggtgccggtcggcgcagcggctgatcaagacgtgcgtgtggccgcgagcaccgcgccatcgaccgacggtaaaagcgtgcaccagaatgcggcgctggacagccgtgtcatgtttgagggttttagcaactttcaagcctttgcaacgaagaaagaagagtacaccaacgtcgtcatcgcgaagaacgtcgataagttcgcggaatggggcgttaccgatttcgaaatggcaccgcagtatgtgtctagcaccgatggctcgtttctggattccgtgatccaaaatggttatgcatttaccgaccgctatgacctgggcattagcaagccgaataagtatggtacggcggatgatctggttaaagcgatcaaggcgctgcattctaaaggtattaaggttatggccgactgggttccagatcagatgtatgctttcccggaaaaagaagtggtgacggccacccgcgtggacaaatatggtacgccggtcgcgggcagccagatcaaaaacactctgtatgtcgtggatggcaaaagctccggtaaagatcagcaagcgaaatatggcggtgccttcctggaagagttgcaggcgaaatacccggaactgttcgcgcgtaagcagatcagcactggtgttccgatggacccgagcgtgaagattaaacaatggtccgcgaaatactttaacggcacgaacatcctgggtcgtggtgccggctacgtgctgaaagaccaggcaacgaatacgtactttagcttggtgtccgacaatacgtttctgccgaagtctctggtcaacccgaaccacggtacgagcagctctgtgaccggcctggtgttcgatggtaagggctacgtgtactactctaccagcggttaccaggccaagaatacgttcatcagcctgggtaacaactggtattacttcgacaataacggttacatggtcacgggtgcgcagagcatcaacggtgccaactactattttctgagcaacggcattcagctgcgtaatgcgatttacgacaatggcaataaggttctgagctactacggtaatgacggtcgtcgttatgagaatggctattacctgtttggccaacagtggcgctactttcaaaatggtattatggccgtcggtctgacccgtgtccacggtgcggtgcagtattttgacgccagcggcttccaagccaagggccagttcatcaccactgcggacggtaaactgcgttactttgaccgtgacagcggcaaccaaatcagcaatcgttttgttcgtaacagcaagggtgaatggtttttgttcgatcataacggcgtggcggttaccggcaccgttactttcaatggtcaacgtctgtactttaagccgaacggtgttcaggcaaagggtgagttcattcgcgacgcggatggtcacttgcgttactacgaccctaattccggtaatgaggttcgtaaccgtttcgtccgcaactctaagggcgaatggttcctgtttgaccacaatggcatcgcagtcaccggcgctcgtgtggtcaacggccaacgcttgtacttcaaaagcaatggcgtccaagctaagggtgagctgattaccgaacgtaagggccgtattaagtattatgatcctaacagcggtaacgaagtgcgtaaccgctacgtccgcaccagcagcggtaattggtactattttggtaacgatggttacgcgctgatcggctggcatgttgttgagggtcgtcgtgtgtactttgatgagaacggtgtctatcgttacgcgagccacgaccagcgtaatcattggaactacgactatcgtcgcgatttcggtcgtggtagcagctccgctatccgttttcgccatagccgtaacggctttttcgacaacttcttccgcttctaactcgagccccaagggcgacacaaaatttattctaaatgataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagtcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgccctt

SEQ ID NO: 16 - protein, 1267 aa, Streptococcus mutans
MVNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNTFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGVYRYASHDQRNHWNYDYRRDFGRGSSSAIRFRHSRNGFFDNFFRF

SEQ ID NO: 17 - protein, 1455 aa, Streptococcus mutans
MEKKVRFKLRKVKKRWVTVSVASAVVTLTSLSGSLVKADSTDDRQQAVTESQASLVTTSEAAKETLTATDTSTATSATSQPTATVTDNVSTTNQSTNTTANTANFDVKPTTTSEQAKTDNSDKIIATSKAVNRLTATGKFVPANNNTAHPKTVTDKIVPIKPKIGKLKQPSSLSQDDIAALGNVKNIRKVNGKYYYYKEDGTLQKNYALNINGKTFFFDETGALSNNTLPSKKGNITNNDNTNSFAQYNQVYSTDAANFEHVDHYLTAESWYRPKYILKNGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRITRTSGVAVIEGNNPSLRLNDTDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNTFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGVYRYASHDQRNHWNYDYRRDFGRGSSSAIRFRHSRNGFFDNFFRF

SEQ ID NO: 18 - DNA, 3804 bp, Streptococcus mutans
  CDS (1)..(3804)
atggtcaatggcaaatactactactacaaagaggacggtacgttgcagaagaactacgcactgaacattaacggcaagacctttttctttgacgagactggcgccctgagcaataacaccctgccgagcaagaaaggtaacatcaccaataacgacaataccaatagcttcgcgcaatacaatcaggtgtattcgacggatgcagcgaacttcgaacatgtcgatcactacctgacggcggagtcctggtatcgcccgaagtatattctgaaaaatggcaagacgtggactcagtccacggagaaagattttcgcccgttgttgatgacctggtggccggatcaggaaacccagcgtcagtatgtaaactatatgaatgcccagctgggtattcaccagacctacaacacggcgaccagcccgttgcaactgaatctggcggcacagacgatccagaccaagattgaagagaagatcacggcggagaagaacactaattggctgcgtcaaacgatttcggcctttgtcaaaacccagagcgcgtggaactcggacagcgaaaaaccgtttgacgatcatctgcaaaagggtgcactgctgtactctaacaatagcaagttgacctctcaagctaatagcaactaccgtattctgaaccgtaccccaaccaaccaaaccggcaagaaagatccgcgttataccgctgaccgtaccatcggtggttatgagttcttgctggcgaacgatgtggataatagcaatcctgttgttcaagcggaacagctgaactggctgcacttcctgatgaactttggcaatatctatgcaaacgaccctgacgccaactttgacagcatccgtgtagacgccgtggacaacgtggatgcagatttgttgcaaatcgctggtgactatctgaaggctgcaaagggcatccataagaacgacaaagcagcgaacgaccacctgtcgatcctggaagcatggagcgataatgacaccccgtatctgcacgacgacggtgacaacatgatcaatatggacaacaagctgcgtctgagcctgctgtttagcctggcgaagccgttgaaccagcgttcgggcatgaacccgctgatcacgaacagcctggttaaccgtaccgatgacaacgcagaaaccgcagcggtcccgagctacagctttatccgtgcacacgatagcgaggttcaagacctgattcgtaacattattcgtgctgagattaatccgaacgtcgtcggttatagcttcacgatggaagagatcaagaaggcctttgagatttacaacaaggatctgctggcgacggaaaagaaatacacccactataacaccgcgctgagctacgcgctgctgctgaccaataagagcagcgttccgcgtgtgtattacggtgatatgtttactgacgacggtcagtacatggcacataaaacgatcaactacgaggctatcgaaacgctgttgaaggcgcgcattaagtacgtgtctggtggccaagcgatgcgtaatcaacaggtgggtaatagcgaaatcattacgagcgtccgctatggcaagggcgcactgaaagcgacggataccggcgatcgtatcacgcgcaccagcggcgttgcggttattgaaggcaataacccgagcctgcgcttgaacgacaccgaccgcgtcgttgttaacatgggtgcagcacacaagaaccaggcatatcgtccgctgttgctgaccactgataatggcatcaaagcgtatcacagcgatcaggaagctgcgggcctggtgcgctataccaatgatcgtggtgaattgatcttcacggcagctgacattaaaggttatgcaaatccgcaagtcagcggttatctgggcgtctgggtgccggtcggcgcagcggctgatcaagacgtgcgtgtggccgcgagcaccgcgccatcgaccgacggtaaaagcgtgcaccagaatgcggcgctggacagccgtgtcatgtttgagggttttagcaactttcaagcctttgcaacgaagaaagaagagtacaccaacgtcgtcatcgcgaagaacgtcgataagttcgcggaatggggcgttaccgatttcgaaatggcaccgcagtatgtgtctagcaccgatggctcgtttctggattccgtgatccaaaatggttatgcatttaccgaccgctatgacctgggcattagcaagccgaataagtatggtacggcggatgatctggttaaagcgatcaaggcgctgcattctaaaggtattaaggttatggccgactgggttccagatcagatgtatgctttcccggaaaaagaagtggtgacggccacccgcgtggacaaatatggtacgccggtcgcgggcagccagatcaaaaacactctgtatgtcgtggatggcaaaagctccggtaaagatcagcaagcgaaatatggcggtgccttcctggaagagttgcaggcgaaatacccggaactgttcgcgcgtaagcagatcagcactggtgttccgatggacccgagcgtgaagattaaacaatggtccgcgaaatactttaacggcacgaacatcctgggtcgtggtgccggctacgtgctgaaagaccaggcaacgaatacgtactttagcttggtgtccgacaatacgtttctgccgaagtctctggtcaacccgaaccacggtacgagcagctctgtgaccggcctggtgttcgatggtaagggctacgtgtactactctaccagcggttaccaggccaagaatacgttcatcagcctgggtaacaactggtattacttcgacaataacggttacatggtcacgggtgcgcagagcatcaacggtgccaactactattttctgagcaacggcattcagctgcgtaatgcgatttacgacaatggcaataaggttctgagctactacggtaatgacggtcgtcgttatgagaatggctattacctgtttggccaacagtggcgctactttcaaaatggtattatggccgtcggtctgacccgtgtccacggtgcggtgcagtattttgacgccagcggcttccaagccaagggccagttcatcaccactgcggacggtaaactgcgttactttgaccgtgacagcggcaaccaaatcagcaatcgttttgttcgtaacagcaagggtgaatggtttttgttcgatcataacggcgtggcggttaccggcaccgttactttcaatggtcaacgtctgtactttaagccgaacggtgttcaggcaaagggtgagttcattcgcgacgcggatggtcacttgcgttactacgaccctaattccggtaatgaggttcgtaaccgtttcgtccgcaactctaagggcgaatggttcctgtttgaccacaatggcatcgcagtcaccggcgctcgtgtggtcaacggccaacgcttgtacttcaaaagcaatggcgtccaagctaagggtgagctgattaccgaacgtaagggccgtattaagtattatgatcctaacagcggtaacgaagtgcgtaaccgctacgtccgcaccagcagcggtaattggtactattttggtaacgatggttacgcgctgatcggctggcatgttgttgagggtcgtcgtgtgtactttgatgagaacggtgtctatcgttacgcgagccacgaccagcgtaatcattggaactacgactatcgtcgcgatttcggtcgtggtagcagctccgctatccgttttcgccatagccgtaacggctttttcgacaacttcttccgcttctaa

SEQ ID NO: 19 - protein, 1267 aa, Streptococcus mutans
MVNGKYYYYKEDGTLQKNYALNINGKTFFFDETGALSNNTLPSKKGNITNNDNTNSFAQYNQVYSTDAANFEHVDHYLTAESWYRPKYILKNGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRITRTSGVAVIEGNNPSLRLNDTDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNTFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGVYRYASHDQRNHWNYDYRRDFGRGSSSAIRFRHSRNGFFDNFFRF

SEQ ID NO: 20 - DNA, 1630 bp, artificial sequence
  synthetic construct
cgcgcaatacaatcaggtgtattcgacggatgcagcgaacttcgaacatgtcgatcactacctgacggcggagtcctggtatcgcccgaagtatattctgaaaaatggcaagacgtggactcagtccacggagaaagattttcgcccgttgttgatgacctggtggccggatcaggaaacccagcgtcagtatgtaaactatatgaatgcccagctgggtattcaccagacctacaacacggcgaccagcccgttgcaactgaatctggcggcacagacgatccagaccaagattgaagagaagatcacggcggagaagaacactaattggctgcgtcaaacgatttcggcctttgtcaaaacccagagcgcgtggaactcggacagcgaaaaaccgtttgacgatcatctgcaaaagggtgcactgctgtactctaacaatagcaagttgacctctcaagctaatagcaactaccgtattctgaaccgtaccccaaccaaccaaaccggcaagaaagatccgcgttataccgctgaccgtaccatcggtggttatgagttcttgctggcgaacgatgtggataatagcaatcctgttgttcaagcggaacagctgaactggctgcacttcctgatgaactttggcaatatctatgcaaacgaccctgacgccaactttgacagcatccgtgtagacgccgtggacaacgtggatgcagatttgttgcaaatcgctggtgactatctgaaggctgcaaagggcatccataagaacgacaaagcagcgaacgaccacctgtcgatcctggaagcatggagcgataatgacaccccgtatctgcacgacgacggtgacaacatgatcaatatggacaacaagctgcgtctgagcctgctgtttagcctggcgaagccgttgaaccagcgttcgggcatgaacccgctgatcacgaacagcctggttaaccgtaccgatgacaacgcagaaaccgcagcggtcccgagctacagctttatccgtgcacacgatagcgaggttcaagacctgattcgtaacattattcgtgctgagattaatccgaacgtcgtcggttatagcttcacgatggaagagatcaagaaggcctttgagatttacaacaaggatctgctggcgacggaaaagaaatacacccactataacaccgcgctgagctacgcgctgctgctgaccaataagagcagcgttccgcgtgtgtattacggtgatatgtttactgacgacggtcagtacatggcacataaaacgatcaactacgaggctatcgaaacgctgttgaaggcgcgcattaagtacgtgtctggtggccaagcgatgcgtaatcaacaggtgggtaatagcgaaatcattacgagcgtccgctatggcaagggcgcactgaaagcgacggataccggcgatcgtatcacgcgcaccagcggcgttgcggttattgaaggcaataacccgagcctgcgcttgaacgacaccgaccgcgtcgttgttaacatgggtgcagcacacaagaaccaggcatatcgtccgctgttgctgaccactgataatggcatcaaagcgtatcacagcgatcaggaagctgcgggcctggtg

SEQ ID NO: 21 - DNA, 28 bp, artificial sequence
  synthetic construct
aatacaatcaggtgtattcgacggatgc

SEQ ID NO: 22 - DNA, 27 bp, artificial sequence
  synthetic construct
tcctgatcgctgtgatacgctttgatg

SEQ ID NO: 23 - DNA, 7790 bp, artificial sequence
  synthetic construct
aattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttaggaggtaaaacatatggtcaatggcaaatactactactacaaagaggacggtacgttgcagaagaactacgcactgaacattaacggcaagacctttttctttgacgagactggcgccctgagcaataacaccctgccgagcaagaaaggtaacatcaccaataacgacaataccaatagcttcgcgcaatacaatcaggtgtattcgacggatgcagcgaacttcgaacatgtcgatcactacctgacggcggagtcctggtatcgcccgaagtatattctgaaaaatggcaagacgtggactcagtccacggagaaagattttcgcccgttgttgatgacctggtggccggatcaggaaacccagcgtcagtatgtaaactatatgaatgcccagctgggtattcaccagacctacaacacggcgaccagcccgttgcaactgaatctggcggcacagacgatccagaccaagattgaagagaagatcacggcggagaagaacactaattggctgcgtcaaacgatttcggcctttgtcaaaacccagagcgcgtggaactcggacagcgaaaaaccgtttgacgatcatctgcaaaagggtgcactgctgtactctaacaatagcaagttgacctctcaagctaatagcaactaccgtattctgaaccgtaccccaaccaaccaaaccggcaagaaagatccgcgttataccgctgaccgtaccatcggtggttatgagttcttgctggcgaacgatgtggataatagcaatcctgttgttcaagcggaacagctgaactggctgcacttcctgatgaactttggcaatatctatgcaaacgaccctgacgccaactttgacagcatccgtgtagacgccgtggacaacgtggatgcagatttgttgcaaatcgctggtgactatctgaaggctgcaaagggcatccataagaacgacaaagcagcgaacgaccacctgtcgatcctggaagcatggagcgataatgacaccccgtatctgcacgacgacggtgacaacatgatcaatatggacaacaagctgcgtctgagcctgctgtttagcctggcgaagccgttgaaccagcgttcgggcatgaacccgctgatcacgaacagcctggttaaccgtaccgatgacaacgcagaaaccgcagcggtcccgagctacagctttatccgtgcacacgatagcgaggttcaagacctgattcgtaacattattcgtgctgagattaatccgaacgtcgtcggttatagcttcacgatggaagagatcaagaaggcctttgagatttacaacaaggatctgctggcgacggaaaagaaatacacccactataacaccgcgctgagctacgcgctgctgctgaccaataagagcagcgttccgcgtgtgtattacggtgatatgtttactgacgacggtcagtacatggcacataaaacgatcaactacgaggctatcgaaacgctgttgaaggcgcgcattaagtacgtgtctggtggccaagcgatgcgtaatcaacaggtgggtaatagcgaaatcattacgagcgtccgctatggcaagggcgcactgaaagcgacggataccggcgatcgtatcacgcgcaccagcggcgttgcggttattgaaggcaataacccgagcctgcgcttgaacgacaccgaccgcgtcgttgttaacatgggtgcagcacacaagaaccaggcatatcgtccgctgttgctgaccactgataatggcatcaaagcgtatcacagcgatcaggaagctgcgggcctggtgcgctataccaatgatcgtggtgaattgatcttcacggcagctgacattaaaggttatgcaaatccgcaagtcagcggttatctgggcgtctgggtgccggtcggcgcagcggctgatcaagacgtgcgtgtggccgcgagcaccgcgccatcgaccgacggtaaaagcgtgcaccagaatgcggcgctggacagccgtgtcatgtttgagggttttagcaactttcaagcctttgcaacgaagaaagaagagtacaccaacgtcgtcatcgcgaagaacgtcgataagttcgcggaatggggcgttaccgatttcgaaatggcaccgcagtatgtgtctagcaccgatggctcgtttctggattccgtgatccaaaatggttatgcatttaccgaccgctatgacctgggcattagcaagccgaataagtatggtacggcggatgatctggttaaagcgatcaaggcgctgcattctaaaggtattaaggttatggccgactgggttccagatcagatgtatgctttcccggaaaaagaagtggtgacggccacccgcgtggacaaatatggtacgccggtcgcgggcagccagatcaaaaacactctgtatgtcgtggatggcaaaagctccggtaaagatcagcaagcgaaatatggcggtgccttcctggaagagttgcaggcgaaatacccggaactgttcgcgcgtaagcagatcagcactggtgttccgatggacccgagcgtgaagattaaacaatggtccgcgaaatactttaacggcacgaacatcctgggtcgtggtgccggctacgtgctgaaagaccaggcaacgaatacgtactttagcttggtgtccgacaatacgtttctgccgaagtctctggtcaacccgaaccacggtacgagcagctctgtgaccggcctggtgttcgatggtaagggctacgtgtactactctaccagcggttaccaggccaagaatacgttcatcagcctgggtaacaactggtattacttcgacaataacggttacatggtcacgggtgcgcagagcatcaacggtgccaactactattttctgagcaacggcattcagctgcgtaatgcgatttacgacaatggcaataaggttctgagctactacggtaatgacggtcgtcgttatgagaatggctattacctgtttggccaacagtggcgctactttcaaaatggtattatggccgtcggtctgacccgtgtccacggtgcggtgcagtattttgacgccagcggcttccaagccaagggccagttcatcaccactgcggacggtaaactgcgttactttgaccgtgacagcggcaaccaaatcagcaatcgttttgttcgtaacagcaagggtgaatggtttttgttcgatcataacggcgtggcggttaccggcaccgttactttcaatggtcaacgtctgtactttaagccgaacggtgttcaggcaaagggtgagttcattcgcgacgcggatggtcacttgcgttactacgaccctaattccggtaatgaggttcgtaaccgtttcgtccgcaactctaagggcgaatggttcctgtttgaccacaatggcatcgcagtcaccggcgctcgtgtggtcaacggccaacgcttgtacttcaaaagcaatggcgtccaagctaagggtgagctgattaccgaacgtaagggccgtattaagtattatgatcctaacagcggtaacgaagtgcgtaaccgctacgtccgcaccagcagcggtaattggtactattttggtaacgatggttacgcgctgatcggctggcatgttgttgagggtcgtcgtgtgtactttgatgagaacggtgtctatcgttacgcgagccacgaccagcgtaatcattggaactacgactatcgtcgcgatttcggtcgtggtagcagctccgctatccgttttcgccatagccgtaacggctttttcgacaacttcttccgcttctaactcgagccccaagggcgacacaaaatttattctaaatgataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagtcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgccctt

SEQ ID NO: 24 - DNA, 36 bp, artificial sequence
  synthetic construct
ataaaaaacgctcggttgccgccgggcgttttttat

SEQ ID NO: 25 - DNA, 21 bp, artificial sequence
  synthetic construct
ggatcctgactgcctgagctt

SEQ ID NO: 26 - DNA, 3801 bp, Streptococcus mutans
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcctttactgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagccgatcgcactatcggcggttacgaatttttgttagccaatgatgtggataattccaatcctgtcgtgcaggccgaacaattgaactggctacattttctcatgaactttggtaacatttatgccaatgatccggatgctaactttgattccattcgtgttgatgcggtagataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcattcgtgctcatgacagtgaagtgcaggacttgattcgcaatattattagagcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagctcataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacggatggttctttcttggattctgtgatccaaaacggctatgcttttacggaccgttatgatttgggaatttccaaacctaataaatacgggacagccgatgatttggtgaaagcaataaaagcgttacacagcaagggtattaaggtaatggctgactgggtgcctgatcaaatgtatgcttttcctgaaaaagaagtggtaacagcaacccgcgttgataagtatgggactcctgttgcaggaagtcagatcaaaaacaccctttatgtagttgatggtaagagttctggtaaagatcaacaagccaagtatgggggagctttcttagaggagctgcaagcgaagtatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggattggtatttgatggtaaaggttatgtttattattcaacgagtggttaccaagccaaaaatactttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtgttcatggtgctgttcaatattttgatgcttctgggttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatggacaacgtctttactttaaacctaatggtgttcaagccaaaggagaatttatcagagatgcagatggacatctaagatattatgatcctaattccggaaatgaagttcgtaatcgctttgttagaaattccaagggagaatggttcttatttgatcacaatggtatcgctgtaactggtgccagagttgttaatggacagcgcctctattttaagtctaatggtgttcaggctaagggagagctcattacagagcgtaaaggtcgtatcaaatactatgatcctaattccggaaatgaagttcgtaatcgttatgtgagaacatcatcaggaaactggtactattttggcaatgatggctatgccttaattggttggcatgttgttgaaggaagacgtgtttactttgatgaaaatggtgtttatcgttatgccagtcatgatcaaagaaaccactggaattatgattacagaagagactttggtcgtggcagcagtagtgctattcgttttagacactctcgtaatggattctttgacaatttctttagattttaa

SEQ ID NO: 27 - DNA, 3801 bp, Streptococcus mutans
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaaatacatcttaaaagatggcaaaacatggacacagtcaacagaaaaagatttccgtcccttactgatgacatggtggcctgaccaagaaacgcagcgtcaatatgttaactacatgaatgcacagcttggtattcatcgaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaaccggaaagaaagatccaaggtatacagctgatcgcactatcggcggttacgaatttcttttggcaaacgatgtggataattctaatcctgtcgtgcaggccgaacaattgaactggctacattttctcatgaactttggtaacatttatgccaatgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagagcagaaatcaatcctaatgttgtcgggtattctttcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagcccataagaaccaagcataccgtccattattgttaactaccaacaatgggattaaagcatatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggggtcacagattttgaaatggcaccgcagtatgtgtcttcaacagatggttctttcttggattctgtgatccaaaacggctatgcttttacggaccgttatgatttaggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagataaaaaacaccctttatgtagttgatggtaagagttctggtaaagatcaacaagccaagtatgggggagctttcttagaggagctgcaagctaaatatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggattggtatttgatggtaaaggttatgtttattattcaacgagtggttaccaagccaaaaatgctttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgccgttatgaaaatggttactatctctttggacaacaatggcgttatttccaaaatggtattatggctgtcggtttaacacgtgttcatggtgctgttcaatactttgatgcttctggcttccaagctaaaggacagtttattacaactgctgatggaaagctgcgttactttgatagagactcaggaaatcaaatttcaaatcgttttgttagaaattccaggggagaatggttcctatttgatcacaatggtgtcgctgtaaccggtactgtaacgttcaatggacaacgtctttactttaaacctaatggtgttcaagccaaaggagaatttatcagagatgcagatggacatctaagatattatgatcctaattccggaaatgaagttcgtaatcgctttgttagaaattccaagggagaatggttcttatttgatcacaatggtatcgctgtaactggtgccagagttgttaacggacagcgcctctattttaagtctaatggtgttcaggctaagggagagctcattacagagcgtaaaggtcgtattaaatattatgatcctaattccggaaatgaagttcgtaatcgttatgtgagaacatcatcaggaaactggtactattttggcaatgatggctatgctttaattggttggcatgttgttgaaggaagacgtgtttactttgatgaaaatggtatttatcgttatgccagtcatgatcaaagaaaccactgggattatgattacagaagagactttggtcgtggcagcagtagtgctattcgttttagacactctcgtaatggattctttgacaatttctttagattttaa

SEQ ID NO: 28 - protein, 1266 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHRTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTNNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNAFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSRGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGIYRYASHDQRNHWDYDYRRDFGRGSSSAIRFRHSRNGFFDNFFRF

SEQ ID NO: 29 - DNA, 3801 bp, Streptococcus mutans
gtgaacggtaaatattattattataaagaagatggaactcttcaaaagaattatgctttaaacattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagccgaaagttggtatcgtcctaagtacatcttgaaggatggcaaaacatggacacagtcaacagaaaaagatttccgtcctttactgatgacatggtggcctgaccaagaaacgcagcgtcaatatgttaactacatgaatgcacagcttggtattcatcaaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagctgataacactatcggcggttacgaatttcttttggcaaacgatgtggataattccaatcctgtcgtgcaggccgaacaattgaactggctccattttctcatgaactttggtaacatttatgccaatgatccggatgctaactttgattccattcgtgttgatgcggtagataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacccttaaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagaacagaaatcaatcctaatgttgtcgggtattctttcactatggaggaaatcaagaaggctttcgagatttacaacaaagacttgttagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaggctcgtattaagtatgtttcaggcggtcaagccatgcgcaatcaacaggttggcaattctgaaattattacgtctgtccgctatggtaaaggtgctttgaaagcaacggatacaggggaccgcaccacacgaacttcaggagtggccgtgattgaaggcaataacccttctttacgtttgaaggcttctgatcgtgttgttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtctgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacagatggttctttcttggattctgtgatccaaaacggctatgcttttacggatcgttatgatttgggaatttccaaacctaataaatacgggacagccgatgatttggttaaggccatcaaagcgttacacagcaagggcattaaggtaatggctgactgggtgcctgatcaaatgtatgctctccctgaaaaagaagtggtaacagcaacccgcgttgataagtatgggactcctgttgcaggaagtcagatcaaaaacaccctttatgtagttgatggtaagagttctggtaaagatcaacaagccaagtatgggggagccttcttagaggagctgcaagcgaagtatccggagctttttgcgagaaagcaaatttccacaggggttccgatggacccttcagttaagattaagcaatggtctgccaagtactttaatgggacaaatattttagggcgcggagcaggctatgtcttaaaagatcaggcaactaatacttacttcagtcttgtttcagacaacaccttccttcctaaatcgttagttaacccaaatcacggaacaagcagttctgtaactggattggtatttgatggtaaaggttatgtttattattcaacgagtggttaccaagccaaaaatgctttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtgttcatggtgctgttcaatattttgatgcttctgggttccaagctaaaggacagtttattacaactgctgatggaaagctgcgttactttgatagagactcaggaaatcaaatttcaaatcgttttgttagaaattctaagggagaatggttcttatttgatcacaatggtgtcgctgtaaccggtactgtaacgttcaatggacaacgtctttactttaaacctaatggtgttcaagccaaaggagaatttatcagagatgcagatggacatctaagatattatgatcctaattccggaaatgaagttcgtaatcgctttgttagaaattccaagggagaatggttcttatttgatcacaatggtatcgctgtaaccggtgccagagttgttaacggacagcgcctctattttaagtctaatggtgttcaggctaagggagagctcattacagagcgtaaaggtcgtatcaaatactatgatcctaattccggaaatgaagttcgtaatcgttatgtgataacatcatcaggaaactggtactattttggcaatgatggctatgctttaattggttggcatattgttgaaggaagacgtgtttattttgatgaaaatggtgtttatcgttatgccagtcatgatcaaagaaaccactgggattatgattacagaagagactttggtcgtggcagcagtagtgctattcgttttagacactctcgtaatggattctttgacaatttctttagattttaa

SEQ ID NO: 30 - protein, 1266 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRTEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNAFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVITSSGNWYYFGNDGYALIGWHIVEGRRVYFDENGVYRYASHDQRNHWDYDYRRDFGRGSSSAIRFRHSRNGFFDNFFRF

SEQ ID NO: 31 - DNA, 3801 bp, Streptococcus mutans
gtgagcggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcccttactgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccatttgatgatcacttacaaaaaggggcattgctttacagtaataatagcaaactaacttcacaggctaattccaactaccgtatcttaaatcgcaccccgaccaatcaaactgggaagaaggacccaaggtatacagctgatcgcaccattggcggttacgaatttcttttggcaaacgatgtggataattccaatcctgtcgtgcaggccgaacaattgaactggctacattttctcatgaactttggtaacatttatgccaatgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagctatgacgacactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagagcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggatcgcaccacacgaacttcaggagtggctgtgattgaaggcaataacccttctttacgtttgaaggcttctgatcgcgtggttgtcaatatgggagcaacccataagaaccaagcataccgacctttacttttgaccacagataacggtatcaaggcttatcattccgatcaagaagcggctggtttggtgcgctacaccaacgacagaggggaattgatcttcacagctgctgatattaaaggctatgccaaccctcaagtttctggctatttaggtgtttgggttccagtaggcgctgccgctgatcaagatgttcgcgttgcggcttcaacggccccatcaacagatggtaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggggtcacagattttgaaatggcaccgcagtatgtgtcttcaacagatggttccttcttggattctgtgatccaaaacggctatgcttttacggaccgttatgatttgggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgcgttgataagtatgggactcctgttgcaggaagtcagatcaaaaacaccctttatgtagttgatggtaagagttctggtaaagatcaacaagccaagtatgggggagctttcttagaagagctgcaagcgaagtatccggagctttttgcgagaaaacaaatttccacaggggttccgatggacccttcagttaagattaagcaatggtctgccaagtactttaatgggacaaatattttagggcgcggagcaggctatgtcttaaaagatcaggcaactaatacttacttcagtcttgtttcagacaacaccttccttcctaaatcgttagttaacccaaatcatggaacaagcagttctgtaactggattggtatttgatggtaaaggttatgtttattattcaacgagtggttaccaagccaaaaatgctttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtgttcatggtgctgttcaatattttgatgcttctgggttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatggacaacgtctttactttaaacctaatggtgttcaagctaaaggagaatttatcagagatgcagatggacatctaagatattatgatcctaattccggaaatgaagttcgtaatcgctttgttagaaattccaagggagaatggttcttatttgatcacaatggtatcgctgtaactggtaccagagttgttaatggacagcgcctctattttaagtctaatggtgttcaggctaagggagagctcattacagagcgtaaaggtcgtatcaaatactatgatcctaattccggaaatgaagttcgtaatcgttatgtgagaacatcatcaggaaactggtactattttggcaatgatggttatgccttaattggttggcatgttgttgaaggaagacgtgtttactttgatgaaaatggtatttatcgttatgccagtcatgatcaaagaaaccactgggattatgattacagaagagactttggtcgtggcagcagcagtgctgttcgttttagacactctcgtaatggattctttgacaatttctttagattttaa

SEQ ID NO: 32 - protein, 1266 aa, Streptococcus mutans
VSGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYDDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGATHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNAFISLGNNWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGTRVVNGQRLYFKSNGVQAKGELITERKGRIKYYDPNSGNEVRNRYVRTSSGNWYYFGNDGYALIGWHVVEGRRVYFDENGIYRYASHDQRNHWDYDYRRDFGRGSSSAVRFRHSRNGFFDNFFRF

SEQ ID NO: 33 - DNA, 3801 bp, Streptococcus mutans
gtgaacggtaaatattattattataaagaagatggaactcttcaaaagaattatgctttaaacattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaaatacatcttaaaagatggcaaaacatggacacagtcaacagaaaaagatttccgtcccttattgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaacaatagcaagctaacttcacaggctaattccaactaccgtatcttaaatcgcaccccaaccaatcaaaccggaaagaaagatccaaggtatacagccgatcgcactatcggcggttacgaatttcttttggcaaacgatgtggataattctaatcctgtcgtgcaggccgaacaattgaactggctccactttctcatgaactttggtaacatttatgccaatgatccggatgctaactttgattccattcgtgttgatgcggtggataatgtggatgctgacttgctccaaattgctggggattacctcaaagccgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagagcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaagctcgtattaagtatgtttcaggcggtcaagccatgcgcaatcaacaggttggcaattctgaaatcattacgtctgtccgctatggtaaaggtgctttgaaagcaacggatacaggggaccgtaccacacggacttcaggagtggccgtgattgaaggcaataacccttctttacgtttgaaggcttctgatcgcgtggttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggggtcacagattttgaaatggcaccgcagtatgtgtcttcaacagatggttctttcttggattctgtgatccaaaacggctatgcttttacggaccgttatgatttgggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagatcaaaaacaccctttatgtagttgatggtaagagttctggtaaagatcaacaagccaagtatgggggagctttcttagaggagctgcaagcgaagtatccggagctttttgcgagaaaacaaatttccacaggggttccgatggacccttcagttaagattaagcaatggtctgccaagtactttaatgggacaaatattttagggcgcggagcaggctatgtcttaaaagaccaggcaaccaatacttacttcagtcttgtttcagacaacaccttccttcctaaatcgttagttaacccaaatcacggaacaagcagttctgtaactggattggtatttgatggtaaaggttatgtttattattcaacgagtggtaaccaagctaaaaatgctttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggacgtcgttatgaaaatggttactatctctttggtcaacaatggcgttatttccaaaatggtattatggctgtcggcttaacacgtattcatggtgctgttcaatactttgatgcttctgggttccaagctaaaggacagtttattacaactgctgatggaaagctgcgttactttgatagagactcaggaaatcaaatttcaaatcgttttgttagaaattccaagggagaatggttcttatttgatcacaatggtatcgctgtaaccggtactgtaacgttcaatggacaacgtctttactttaaacctaatggtgttcaagccaaaggagaatttatcagagatacagatggacatctaagatattatgatcctaattccggaaatgaagttcgtaatcgctttgttagaaattccaagggagaatggttcttatttgatcacaatggtatcgctgtaactggtgccagagttgttaatggacagcgcctctattttaagtctaatggtgttcaggctaagggagagctcattacagagcgtaaaggtcgtatcaaatactatgatcctaattccggaaatgaagttcgtaatcgttatgtgagaacgtcatcaggaaactggtactattttggcaatgatggctatgccttaattggttggcatgttgttgaaggaagacgtgtttactttgatgaaaatggtgtttatcgttattccagtcatgatcaaagaaaccactgggattatgattacagaagagactttggtcgtggcagcagtagtggtattcgttttagacaccctcgtaatggattctttgacaatttctttagattttaa

SEQ ID NO: 34 - protein, 1266 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGNQAKNAFISLGNNWYYFDNNGYMVTGAQSINGANYYFLSNGIQLRNAIYDNGNKVLSYYGNDGRRYENGYYLFGQQWRYFQNGIMAVGLTRIHGAVQYFDASGFQAKGQFITTADGKLRYFDRDSGNQISNRFVRNSKGEWFLFDHNGIAVTGTVTFNGQRLYFKPNGVQAKGEFIRDTDGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGVYRYSSHDQRNHWDYDYRRDFGRGSSSGIRFRHPRNGFFDNFFRF

SEQ ID NO: 35 - DNA, 3801 bp, Streptococcus mutans
gtgaacggtaaatattattattataaagaagatggaactcttcaaaagaattatgctttaaacattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacgtcttgaagaatggtaaaacatggacacagtcaacagaaaaagattttcgtcccttactgatgacatggtggcctgaccaagaaacgcagcgtcaatatgttaactacatgaatggacagcttggtattcatcaaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagctgatcgcaccattggcggttacgaatttttgttagccaatgatgtggataattctaatcctgtcgtgcaggccgaacagctgaactggctccactttcttatgaactttggtaacatttatgccaatgatccggatgctaactttgattccattcgtgttgatgcggtggataatgtggatgctgacttactccaaattgctggggattacctcaaagctgctaaggggattcataaaaatgataaggctgctaatgatcatttatctattttagaggcatggagtgacaacgacactccttaccttcatgatgatggcgacaatatgattaacatggataacaggttacgtctttccttgctttattcattagctaaacccttaaatcaacgttcaggcatgaatcctctgatcactaacagtctggtgaatcgaactgatgataatgctgaaactgccgcagtcccttcttattcctacatccgtgcccatgacagtgaagtgcaggacttgattcgcaatattattagagcagaaatcaatcctaatgttgtcgggtattctttcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggctagtacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagactttgaaatggcaccgcagtatgtgtcttcaacggatggttctttcttggattctgtgatccaaaacggctatgcttttacggaccgttatgatttgggaatttccaaacctaataaatacgggacagccgatgatttggtgaaagcaatcaaagcgttacacagcaagggcattaaggtaatggctgactgggtacctgatcaaatgtatgctttccctgaaaaagaagtggtaacagcaacccgtgttgataagtatgggactcctgttgcaggaagtcagatcaaaaacaccctttatgtagttgatggtaagagttctggtaaagatcaacaagccaagtatgggggagctttcttagaggaactgcaagctaagtatccggagctttttgcgagaaagcaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggatttgtatttgatggtaaaggttatgtttattattcaacgagtggttaccaagccaaaaatgctttcatcagcgaaggtgataaatggtattattttgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtgttcatggtgctgttcaatactttgatgcttctggcttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatagacaacgtctttactttaaacctaatggtgttcaagccaaaggagaatttatcagagatgcagatggacatctaagatattatgatcctaattccggaaatgaagttcgtaatcgctttgttagaaattccaagggagaatggttcttatttgatcacaatggtatcgctgtaactggtgccagagttgttaatggacagcgcctctattttaagtctaatggtgttcaggctaagggagagctcattacagagcgtaaaggtcgtatcaaatactatgatcctaattccggaaatgaagttcgtaatcgttatgtgagaacatcatcaggaaactggtactattttggcaatgatggctatgccttaattggttggcatgttgttgaaggaagacgtgtttactttgatgaaaatggtgtttatcgttatgccagtcatgatcaaagaaaccactggaattatgattacagaagagactttggtcgtggcagcagtagtgctattcgttttagacactctcgtaatggattctttgacaatttctttagattttaa

SEQ ID NO: 36 - protein, 1266 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYVLKNGKTWTQSTEKDFRPLLMTWWPDQETQRQYVNYMNGQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNRLRLSLLYSLAKPLNQRSGMNPLITNSLVNRTDDNAETAAVPSYSYIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGFVFDGKGYVYYSTSGYQAKNAFISEGDKWYYFDNNGYMVTGAQSINGANYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNRQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVRTSSGNWYYFGNDGYALIGWHVVEGRRVYFDENGVYRYASHDQRNHWNYDYRRDFGRGSSSAIRFRHSRNGFFDNFFRF

SEQ ID NO: 37 - DNA, 3801 bp, Streptococcus mutans
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcccttattgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccatttgatgatcacttacaaaaaggggcattgctttacagtaataatagcaaactaacttcacaggctaattccaactaccgtatcttaaatcgcaccccgaccaatcaaactgggaagaaggacccaagatatacagctgataacactatcggcggttacgaatttcttttggcaaacgatgtggataattccaatcctgtcgtgcaggccgaacaattgaactggctccactttctcatgaactttggcaacatttatgccaatgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagtgacaacgacactccttaccttcatgatgatggcgacaatatgattaatatggacaataagctgcgtttgtctctattattttcattagctaaacctttaaatcaacgttcaggcatgaatcctctgatcactaacagtttggtgaatcgaactgatgataatgctgaaactgccgcagtcccttcttattccttcatccgtgcccatgacagtgaagtgcaggatttgattcgtgatatcatcaaggcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaagctcgtattaagtatgtttcaggcggtcaagccatgcgcaatcaacaggttggcaattctgaaattattacgtctgtccgctatggtaaaggtgctttgaaagcaacggatacaggggaccgcaccacacgaacttcaggagtggccgtgattgaaggcaataacccttctttacgtttgaaggcttctgatcgcgtggttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacagatggttctttcttggattctgtgatccaaaacggctatgcttttacggaccgttatgacttaggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagatcaaaaacaccctttatgtagttgatggtaagagttctggtaaagatcaacaagccaagtatgggggagctttcttagaggagctgcaagctaaatatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggattggtatttgatggtaaaggttatgtttattattcaacgagtggtaaccaagctaaaaatgctttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtattcatggtgctgttcaatactttgatgcttctgggttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatggacaacgtctttactttaaacctaatggtgttcaagccaaaggagaatttatcagagatgcagatggacatctaagatattatgatcctaattccggaaatgaagttcgtaatcgctttgttagaaattccaagggagaatggttcttatttgatcacaatggtatcgctgtaactggtaccagagttgttaatggacagcgcctctattttaagtctaatggtgttcaggctaagggagagctcattacagagcgtaaaggtcgtatcaaatactatgatcctaattccggaaatgaagttcgtaatcgttatgtgagaacgtcatcaggaaactggtactattttggcaatgatggctatgccttaattggttggcatgttgttgaaggaagacgtgtttactttgatgaaaatggtgtttatcgttatgccagtcatgatcaaagaaaccactgggattatgattacagaagagactttggtcgtggcagcagcagtgctgttcgttttagacactctcgtaatggattctttgacaatttctttagattttaa

SEQ ID NO: 38 - protein, 1266 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGNQAKNAFISLGNNWYYFDNNGYMVTGAQSINGANYYFLSNGIQLRNAIYDNGNKVLSYYGNDGRRYENGYYLFGQQWRYFQNGIMAVGLTRIHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGTRVVNGQRLYFKSNGVQAKGELITERKGRIKYYDPNSGNEVRNRYVRTSSGNWYYFGNDGYALIGWHVVEGRRVYFDENGVYRYASHDQRNHWDYDYRRDFGRGSSSAVRFRHSRNGFFDNFFRF

SEQ ID NO: 39 - DNA, 3801 bp, Streptococcus mutans
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcccttattgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaacaatagcaagctaacttcacaggctaattccaactaccgtatcttaaatcgcaccccaaccaatcaaaccggaaagaaagatccaaggtatacagccgatcgcaccatcggtggttacgagttcttgctggctaatgatgtggataattccaatcctgttgttcaggccgaacagctgaactggctccactttcttatgaactttggtaacatttatgccaacgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtctggtgaatcgaactgatgataatgctgaaactgccgcagtcccttcttattccttcatccgtgcccatgacagtgaagtgcaggacttgattcgtgatatcatcaaggcagaaatcaatcctaatgttgtcgggtattctttcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaagctcgtattaagtatgtttcaggcggtcaagccatgcgcaatcaacaggttggtaattctgaaatcattacgtctgtccgctatggtaaaggtgctttgaaagcaacggatacaggggaccgcatcacacgcacttcaggagtggtcgtgattgaaggcaataacccttctttacgtttgaaggcttctgatcgcgtggttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtctgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggggtcacagattttgaaatggcaccgcagtatgtgtcttcaacagatggttctttcttggattctgtgatccaaaacggctatgcttttacggaccgttatgatttgggaatttccaaacctaataaatacgggacagccgatgatttggtgaaggccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagatcaaaaacaccctttatgtagttgatggtaagagttctggtaaagatcaacaagccaagtatgggggagctttcttagaggagctgcaagctaaatatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggattggtatttgatggtaaaggttatgtttattattcaacgagtggtaaccaagctaaaaatgctttcattagtttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtgttcatggtgctattcaatattttgatgcttctgggttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatggacaacgtctttactttaaacctaatggtgttcaagccaaaggagaatttatcagagatgcaaatggatatctaagatattatgatcctaattccggaaatgaagttcgtaatcgctttgttagaaattccaagggagaatggttcttatttgatcacaatggtgtcgctgtaaccggtgccagagttgttaacggacagcgcctctattttaagtctaatggtgttcaggctaagggagagctcattacagagcgtaaaggtcgtatcaaatactatgatcctaattccggaaatgaagttcgtaatcgttatgtgaaaacatcatcaggaaactggtactattttggcaatgatggctatgccttaattggttggcatattgttgaaggaagacgtgtttactttgatgaaaatggtgtttatcgttatgccagtcatgatcaaagaaaccactgggattatgattacagaagagactttggtcgtggcagcagtagtgctattcgttttagacaccctcgtaatggattctttgacaatttctttagattttaa

SEQ ID NO: 40 - protein, 1266 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRITRTSGVVVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGNQAKNAFISLGNNWYYFDNNGYMVTGAQSINGANYYFLSNGIQLRNAIYDNGNKVLSYYGNDGRRYENGYYLFGQQWRYFQNGIMAVGLTRVHGAIQYFDASGFQAKGQFITTADGKLRYFDRDSGNQISNRFVRNSKGEWFLFDHNGVAVTGTVTFNGQRLYFKPNGVQAKGEFIRDANGYLRYYDPNSGNEVRNRFVRNSKGEWFLFDHNGVAVTGARVVNGQRLYFKSNGVQAKGELITERKGRIKYYDPNSGNEVRNRYVKTSSGNWYYFGNDGYALIGWHIVEGRRVYFDENGVYRYASHDQRNHWDYDYRRDFGRGSSSAIRFRHPRNGFFDNFFRF

SEQ ID NO: 41 - DNA, 3801 bp, Streptococcus mutans
gtgaacggtaaatattattattataaagaagatggaactcttcaaaagaattatgctttaaacattaatgggaaaactttcttctttgatgaaacaggagcattatcaaataatactttacctagtaaaaagggtaatatcactaataatgataacactaacagctttgctcaatataatcaggtctatagtacagatgctacaaacttcgaacatgttgatcattatttgacagctgagagttggtatcgtcctaagtacatcttgaaagatggtaaaacatggacacagtcagcagaaaaagatttccgtcctttactgatgacatggtggcctgaccaagaaacgcagcgtcaatatgttaactacatgaatgcacagcttggtattcatcaaacatacaatacagcaacttcaccgcttcaattgaatttagctgctcagacaatacaaactaaaatcgaagaaaaaatcactgcagaaaagaataccaattggctgcgtcagactatttccgcatttgttaagacacagtcagcttggaacagtgatagcgaaaaaccgtttgatgatcacttacaaaaaggggcattgctttacgataatgaaggaaaattaacgccttatgctaattccaactaccgtatcttaaatcgcaccccgaccaatcaaactgggaagaaggacccaaggtatacagctgatcgcaccattggcggttacgaatttttgttagccaacgatgtggataattccaatcctgtcgtgcaagccgaacaattgaactggctgcattttctcatgaactttggtaacatttatgccaacgatccggatgctaactttgattccattcgtgttgatgcggtggataatgtggatgctgacttactccaaattgctggggattacctcaaagctgctaaagggattcataaaaatgataaggctgctaatgatcatttgtctattttagaggcatggagtgacaacgacactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcatccgtgcccatgacagtgaagtgcaggatttgattcgtgatatcatcaaggcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaagctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagactttgaaatggcaccgcagtatgtgtcttcaacagatggttctttcttggattctgtgatccaaaacggctatgcttttacggaccgttatgatttgggaatttccaaacctaataaatacgggacagccgatgatttggttaaggccatcaaagcgttacacagcaagggcattaaggtaatggctgactgggtgcctgatcaaatgtatgctttccctgaaaaagaagtggtaacagcaacccgcgttgataagtatgggactcctgttgcaggaagtcagatcaaaaacaccctttatgtagttgatggtaagagttctggtaaagatcaacaagccaagtatgggggagctttcttagaggagctgcaagcgaagtatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggattggtatttgatggtaaaggttatgtttattattcaacgagtggttaccaagccaaaaatactttcattagcttaggaaataattggtattatttcgataataacggttatatggtcactggtgctcaatcaattaacggtgttaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtgttcatggtgctgttcaatactttgatgcttctggcttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatggacaacgtctttactttaaacctaatggtgttcaagccaaaggagaatttatcagagatgcagatggacatctaagatattatgatcctaattccggaaatgaagttcgtaatcgctttgttagaaattccaagggagaatggttcttatttgatcacaatggtatcgctgtaactggtgccagagttgttaatggacagcgcctctattttaagtctaatggtgttcaggctaagggagagctcattacagagcgtaaaggtcgtatcaaatactatgatcctaattccggaaatgaagttcgtaatcgttatgtgaaaacatcatcaggaaactggtactattttggcaatgatggctatgctttaattggttggcatattgttgaaggaagacgtgtttattttgatgaaaatggtgtttatcgttatgccagtcatgatcaaagaaaccactgggattatgattacagaagaaactttggtcgtggcagcagtagtgctattcgttttagacactctcgtaatggattctttgacaatttctttagattttaa

SEQ ID NO: 42 - protein, 1266 aa, Streptococcus mutans
VNGKYYYYKEDGTLQKNYALNINGKTFFFDETGALSNNTLPSKKGNITNNDNTNSFAQYNQVYSTDATNFEHVDHYLTAESWYRPKYILKDGKTWTQSAEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYDNEGKLTPYANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNRLRLSLLYSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGYQAKNTFISLGNNWYYFDNNGYMVTGAQSINGVNYYFLSNGIQLRNAIYDNGNKVLSYYGNDGRRYENGYYLFGQQWRYFQNGIMAVGLTRVHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGARVVNGQRLYFKSNGVQAKGELITERKGRIKYYDPNSGNEVRNRYVKTSSGNWYYFGNDGYALIGWHIVEGRRVYFDENGVYRYASHDQRNHWDYDYRRNFGRGSSSAIRFRHSRNGFFDNFFRF

SEQ ID NO: 43 - DNA, 3606 bp, Streptococcus mutans
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcccttattgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccatttgatgatcacttacaaaaaggggcattgctttacagtaataatagcaaactaacttcacaggctaattccaactaccgtatcttaaatcgcaccccgaccaatcaaactgggaagaaggacccaaggtatacagctgatcgcaccattggcggttacgaatttcttttggcaaacgatgtggataattccaatcctgtcgtgcaggccgaacaattgaactggctgcattttctcatgaactttggcaacatttatgccaatgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagtgacaacgacactccttaccttcatgatgatggcgacaatatgattaatatggacaataagctgcgtttgtctctattattttcattagctaaacccttaaatcaacgttcaggcatgaatcctctgatcactaacagtttggtgaatcgaactgatgataatgctgaaactgccgcagtcccttcttattccttcatccgtgcccatgacagtgaagtgcaggatttgattcgtgatatcatcaaggcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaagctcgtattaagtatgtttcaggcggtcaagccatgcgcaatcaacaggttggcaattctgaaatcattacgtctgtccgctatggtaaaggtgctttgaaagcaacggatacaggggaccgtaccacacggacttcaggagtggccgtgattgaaggcaataacccttctttacgtttgaaggcttctgatcgcgtggttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacagatggttctttcttggattctgtgatccaaaacggctatgcttttacggaccgttatgacttaggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagatcaaaaacaccctttatgtagttgatggtaagagttctggtaaagatcaacaagccaagtatgggggagctttcttagaggagctgcaagctaaatatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttctgtaactggattggtatttgatggtaaaggttatgtttattattcaacgagtggtaaccaagctaaaaatgctttcattagcttaggaaataattggtattatttcgataataacggttatatggtcactggtgctcaatcaattaacggtgctaattattatttcttatcaaatggtattcaattaagaaatgctatttatgataatggtaataaagtattgtcttattatggaaatgatggccgtcgttatgaaaatggttactatctctttggtcaacaatggcgttatttccaaaatggtattatggctgtcggcttaacacgtattcatggtgctgttcaatactttgatgcttctgggttccaagctaaaggacagtttattacaactgctgatggaaagctgcgttactttgatagagactcaggaaatcaaatttcaaatcgttttgttagaaattccaagggagaatggttcttatttgatcacaatggtgtcgctgtaaccggtactgtaacgttcaatggacaacgtctttactttaaacctaatggtgttcaagccaaaggagaatttatcagagatgcagatggacatctaagatattatgatcctaattccggaaatgaagttcgtaatcgttatgtgagaacgtcatcaggaaactggtactattttggcaatgatggctatgccttaattggttggcatgttgttgaaggaagacgtgtttactttgatgaaaatggtgtttatcgttatgccagtcatgatcaaagaaaccactgggattatgattacagaagagactttggtcgtggcagcagcagtgctgttcgttttagacactctcgtaatggattctttgacaatttctttagattttaa

SEQ ID NO: 44 - protein, 1201 aa, Streptococcus mutans
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGNQAKNAFISLGNNWYYFDNNGYMVTGAQSINGANYYFLSNGIQLRNAIYDNGNKVLSYYGNDGRRYENGYYLFGQQWRYFQNGIMAVGLTRIHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRYVRTSSGNWYYFGNDGYALIGWHVVEGRRVYFDENGVYRYASHDQRNHWDYDYRRDFGRGSSSAVRFRHSRNGFFDNFFRF

SEQ ID NO: 45 - DNA, 3801 bp, Streptococcus troglodytae
gtgaacggtaaatattattattataaagaagatggaactcttcaaaagaactatgctttaaacattaatgggaaaactttcttctttgatgaaacgggagcattatcaaataatactttacctagtaaaaagggtaatatcactaataatgataacactaatagctttgctcaatataatcaggtctatagtacagatgctgcaaacttcgaacatgttgatcattatttgacagctgagagttggtatcgtcctaagtacatcttgaaagatggtaaaacatggacacagtcaacagaaaaagatttccgtcctttattgatgacatggtggcctgaccaagaaacacagcgtcaatatgtcaactacatgaatgcacagcttgggatcaagcaaacatacaatacagcaaccagtccgcttcaattaaatttagcggctcagacaatacaaactaagatcgaagaaaagatcactgcagaaaagaataccaattggctgcgtcagactatttcagcatttgttaagacacagtcagcttggaatagtgagagcgaaaaaccgtttgatgatcacttacaaaaaggggcattgctttacagtaacaatagcaagctaacttcacaggctaattccaactaccgtattttaaatcgcaccccgaccaatcaaaccggaaagaaagatccacggtatacagccgatcgcaccatcggtggttacgagttcttgctggctaatgatgtggataattccaatcctgttgttcaggccgaacagctgaactggctgcattttctcatgaactttggtaacatttatgccaacgatcctgatgctaactttgattccattcgtgttgatgcggtggacaatgtggatgctgacttacttcaaatcgctggtgattacctcaaagctgctaaagggattcataaaaatgataaggctgccaatgatcatttgtctattttagaggcatggagctataacgacactccttaccttcatgatgatggcgataatatgattaacatggacaatagattacgtctttccttgctttattcattagctaaacccttgaatcaacgttcaggcatgaatcctctcatcactaacagtctggtgaatcgaacagatgataacgctgaaactgccgcagtcccttcttattccttcattcgtgcccatgacagtgaagtgcaggatttgattcgcaatattattagagcagaaatcaatcctaatgttgttggttattctttcaccatggaggaaatcaagaaggctttcgagatttacaacaaagacttactggctacagagaagaaatacacacactataatacggcactttcttatgccctgcttttaactaacaaatccagtgtgccgcgtgtctattacggcgatatgttcacagatgacggtcagtacatggcacataagaccattaattacgaagccatcgaaactctgcttaaagcacggattaagtatgtttcaggcggtcaggccatgcgaaaccaaagtgttggcaattctgaaatcattacgtctgttcgctatggtaagggagccctgaaagcaacggatacaggagaccgcaccacacgcacttctggagtggccgtgattgaaggcaatagcccttctttacgtttgcgttcttatgatcgtgttgttgtcaatatgggagctgcccataagaaccaagcataccgacctttactcttgaccacagataacggtatcaaggcttatcattctgatcaagaagcggctggtttggtgcgctacaccaatgacagaggggaattgatcttcacagcggctgatatcaaaggctatgccaaccctcaagtttctggctatttaggtgtttgggtgccagtaggagctgcagctgatcaagatgtccgtgtggcagccagcactgccccatcaacagacggcaaatcagtgcatcaaaatgcagcccttgattctcgtgtcatgtttgaaggcttctcaaatttccaagcatttgcgactacaaaagaagagtatacgaatgtggtcattgctaagaatgtggataagtttgcggaatggggtgttacagactttgaaatggcaccgcaatatgtgtcttcaacagatggttctttcttggattctgtaattcaaaatggctatgcctttacggatcgttatgatctgggaatttccaaacctaataaatacgggacagccgatgatttggttaaggccatcaaagcattgcacagcaagggcattaaggttatggccgactgggtgcctgatcaaatgtatgctttccctgagaaagaagtggttgaagtcactcgtgtggacaaatatggacatcctgttgcaggcagtcaaatcaaaaacacactttatgtagttgatggtaagagttccggaaaggaccagcaggctaagtatgggggagctttcttagaagagctgcaagctaaatatccagagctctttgccagaaagcaaatttcaacaggggttccgatggacccaactgttaagattaagcaatggtctgccaagtactttaatggaacaaacattttagggcggggagcaggctatgtcttaaaggatcaggcaaccaatacttatttcagtcttgctgcagataataccttccttccgaaatcattagttaatccggatcatggaacgagcagttctgtaataggattagtgtatgatggtaaaggctatacttatcattcaacaagcggcaaccaagctaaaaatgctttcattagcttaggaaataattggtattatttcgataacaacggctatatggtcactggtgctagaactattaacggtgctaattattatttcttatcaaatggtattcaattgagaaatgctatttatgataatggtaataaaatattgtcttattatggaaatgacggtcgccgttatgaaaatggttattatctctttggtcaacaatggcgttatttccaaaatggtgttatggctgtcggcttaacacgtgttcatggtgctgttcaatactttgatgcttctgggttccaagctaaaggacagtttattacaactgctgatggaaagctgcattattttgatagagactcaggaaatcaaatttcaaatcgttttgttagaaattccaagggagagtggttcttatttgatcacaatggtgtcgctgtaactggtacgataacgttcaatggacaacgtctttactttaaacctaatggtgttcaagctaaaggagaatttatcagagatgcaaatggatatctaagatattatgatcctaattccggaaatgaagttcgtaatcgttttgttagaaattccaagggagaatggttcttatttgatcacaatggtatcgctgcaactggtgccagagttgttaacggacaacgcctctactttaagtctaatggtgttcaagctaagggtgagctcattacagagcgtaaaggtcgtattaaatattatgatcctaattctggaaatgaagttcgtaatcgttatgtgagaacatcatcaggaaactggtactattttggtaatgatggttatgccttaattggttggcatgttgttgaaggaagacgtgtttactttgatgaaaatggtatttatcgttatgccagtcatgatcaaagaaaccactgggattatgattacagaagagactttggtcgtggcagcagcagtgctgttcgttttagacaccctcgtaatggattctttgacaatttctttagattttaa

SEQ ID NO: 46 - protein, 1266 aa, Streptococcus troglodytae
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIKQTYNTATSPLQLNLAAQTIQTKIEEKITAEKNTNWLRQTISAFVKTQSAWNSESEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQSVGNSEIITSVRYGKGALKATDTGDRTTRTSGVAVIEGNSPSLRLRSYDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATTKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVEVTRVDKYGHPVAGSQIKNTLYVVDGKSSGKDQQAKYGGAFLEELQAKYPELFARKQISTGVPMDPTVKIKQWSAKYFNGTNILGRGAGYVLKDQATNTYFSLAADNTFLPKSLVNPDHGTSSSVIGLVYDGKGYTYHSTSGNQAKNAFISLGNNWYYFDNNGYMVTGARTINGANYYFLSNGIQLRNAIYDNGNKILSYYGNDGRRYENGYYLFGQQWRYFQNGVMAVGLTRVHGAVQYFDASGFQAKGQFITTADGKLHYFDRDSGNQISNRFVRNSKGEWFLFDHNGVAVTGTITFNGQRLYFKPNGVQAKGEFIRDANGYLRYYDPNSGNEVRNRFVRNSKGEWFLFDHNGIAATGARVVNGQRLYFKSNGVQAKGELITERKGRIKYYDPNSGNEVRNRYVRTSSGNWYYFGNDGYALIGWHVVEGRRVYFDENGIYRYASHDQRNHWDYDYRRDFGRGSSSAVRFRHPRNGFFDNFFRF

SEQ ID NO: 47 - DNA, 2715 bp, artificial sequence
  T1 C-terminal truncation
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcctttactgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagccgatcgcactatcggcggttacgaatttttgttagccaatgatgtggataattccaatcctgtcgtgcaggccgaacaattgaactggctacattttctcatgaactttggtaacatttatgccaatgatccggatgctaactttgattccattcgtgttgatgcggtagataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcattcgtgctcatgacagtgaagtgcaggacttgattcgcaatattattagagcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagctcataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacggatggttctttcttggattctgtgatccaaaacggctatgcttttacggaccgttatgatttgggaatttccaaacctaataaatacgggacagccgatgatttggtgaaagcaataaaagcgttacacagcaagggtattaaggtaatggctgactgggtgcctgatcaaatgtatgcttttcctgaaaaagaagtggtaacagcaacccgcgttgataagtatgggactcctgttgcaggaagtcagatcaaaaacaccctttatgtagttgatggtaagagttctggtaaagatcaacaagccaagtatgggggagctttcttagaggagctgcaagcgaagtatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttaa

SEQ ID NO: 48 - protein, 904 aa, artificial sequence
  T1 C-terminal truncation
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSS

SEQ ID NO: 49 - DNA, 2715 bp, artificial sequence
  T1 C-terminal truncation
gtgaacggtaaatattattattataaagaagatggaactcttcaaaagaattatgctttaaacattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagccgaaagttggtatcgtcctaagtacatcttgaaggatggcaaaacatggacacagtcaacagaaaaagatttccgtcctttactgatgacatggtggcctgaccaagaaacgcagcgtcaatatgttaactacatgaatgcacagcttggtattcatcaaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagctgataacactatcggcggttacgaatttcttttggcaaacgatgtggataattccaatcctgtcgtgcaggccgaacaattgaactggctccattttctcatgaactttggtaacatttatgccaatgatccggatgctaactttgattccattcgtgttgatgcggtagataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacccttaaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagaacagaaatcaatcctaatgttgtcgggtattctttcactatggaggaaatcaagaaggctttcgagatttacaacaaagacttgttagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaggctcgtattaagtatgtttcaggcggtcaagccatgcgcaatcaacaggttggcaattctgaaattattacgtctgtccgctatggtaaaggtgctttgaaagcaacggatacaggggaccgcaccacacgaacttcaggagtggccgtgattgaaggcaataacccttctttacgtttgaaggcttctgatcgtgttgttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtctgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacagatggttctttcttggattctgtgatccaaaacggctatgcttttacggatcgttatgatttgggaatttccaaacctaataaatacgggacagccgatgatttggttaaggccatcaaagcgttacacagcaagggcattaaggtaatggctgactgggtgcctgatcaaatgtatgctctccctgaaaaagaagtggtaacagcaacccgcgttgataagtatgggactcctgttgcaggaagtcagatcaaaaacaccctttatgtagttgatggtaagagttctggtaaagatcaacaagccaagtatgggggagccttcttagaggagctgcaagcgaagtatccggagctttttgcgagaaagcaaatttccacaggggttccgatggacccttcagttaagattaagcaatggtctgccaagtactttaatgggacaaatattttagggcgcggagcaggctatgtcttaaaagatcaggcaactaatacttacttcagtcttgtttcagacaacaccttccttcctaaatcgttagttaacccaaatcacggaacaagcagttaa

SEQ ID NO: 50 - protein, 904 aa, artificial sequence
  T1 C-terminal truncation
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRTEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSS

SEQ ID NO: 51 - DNA, 2715 bp, artificial sequence
  T1 C-terminal truncation
gtgaacggtaaatattattattataaagaagatggaactcttcaaaagaactatgctttaaacattaatgggaaaactttcttctttgatgaaacgggagcattatcaaataatactttacctagtaaaaagggtaatatcactaataatgataacactaatagctttgctcaatataatcaggtctatagtacagatgctgcaaacttcgaacatgttgatcattatttgacagctgagagttggtatcgtcctaagtacatcttgaaagatggtaaaacatggacacagtcaacagaaaaagatttccgtcctttattgatgacatggtggcctgaccaagaaacacagcgtcaatatgtcaactacatgaatgcacagcttgggatcaagcaaacatacaatacagcaaccagtccgcttcaattaaatttagcggctcagacaatacaaactaagatcgaagaaaagatcactgcagaaaagaataccaattggctgcgtcagactatttcagcatttgttaagacacagtcagcttggaatagtgagagcgaaaaaccgtttgatgatcacttacaaaaaggggcattgctttacagtaacaatagcaagctaacttcacaggctaattccaactaccgtattttaaatcgcaccccgaccaatcaaaccggaaagaaagatccacggtatacagccgatcgcaccatcggtggttacgagttcttgctggctaatgatgtggataattccaatcctgttgttcaggccgaacagctgaactggctgcattttctcatgaactttggtaacatttatgccaacgatcctgatgctaactttgattccattcgtgttgatgcggtggacaatgtggatgctgacttacttcaaatcgctggtgattacctcaaagctgctaaagggattcataaaaatgataaggctgccaatgatcatttgtctattttagaggcatggagctataacgacactccttaccttcatgatgatggcgataatatgattaacatggacaatagattacgtctttccttgctttattcattagctaaacccttgaatcaacgttcaggcatgaatcctctcatcactaacagtctggtgaatcgaacagatgataacgctgaaactgccgcagtcccttcttattccttcattcgtgcccatgacagtgaagtgcaggatttgattcgcaatattattagagcagaaatcaatcctaatgttgttggttattctttcaccatggaggaaatcaagaaggctttcgagatttacaacaaagacttactggctacagagaagaaatacacacactataatacggcactttcttatgccctgcttttaactaacaaatccagtgtgccgcgtgtctattacggcgatatgttcacagatgacggtcagtacatggcacataagaccattaattacgaagccatcgaaactctgcttaaagcacggattaagtatgtttcaggcggtcaggccatgcgaaaccaaagtgttggcaattctgaaatcattacgtctgttcgctatggtaagggagccctgaaagcaacggatacaggagaccgcaccacacgcacttctggagtggccgtgattgaaggcaatagcccttctttacgtttgcgttcttatgatcgtgttgttgtcaatatgggagctgcccataagaaccaagcataccgacctttactcttgaccacagataacggtatcaaggcttatcattctgatcaagaagcggctggtttggtgcgctacaccaatgacagaggggaattgatcttcacagcggctgatatcaaaggctatgccaaccctcaagtttctggctatttaggtgtttgggtgccagtaggagctgcagctgatcaagatgtccgtgtggcagccagcactgccccatcaacagacggcaaatcagtgcatcaaaatgcagcccttgattctcgtgtcatgtttgaaggcttctcaaatttccaagcatttgcgactacaaaagaagagtatacgaatgtggtcattgctaagaatgtggataagtttgcggaatggggtgttacagactttgaaatggcaccgcaatatgtgtcttcaacagatggttctttcttggattctgtaattcaaaatggctatgcctttacggatcgttatgatctgggaatttccaaacctaataaatacgggacagccgatgatttggttaaggccatcaaagcattgcacagcaagggcattaaggttatggccgactgggtgcctgatcaaatgtatgctttccctgagaaagaagtggttgaagtcactcgtgtggacaaatatggacatcctgttgcaggcagtcaaatcaaaaacacactttatgtagttgatggtaagagttccggaaaggaccagcaggctaagtatgggggagctttcttagaagagctgcaagctaaatatccagagctctttgccagaaagcaaatttcaacaggggttccgatggacccaactgttaagattaagcaatggtctgccaagtactttaatggaacaaacattttagggcggggagcaggctatgtcttaaaggatcaggcaaccaatacttatttcagtcttgctgcagataataccttccttccgaaatcattagttaatccggatcatggaacgagcagttaa

SEQ ID NO: 52 - protein, 904 aa, artificial sequence
  T1 C-terminal truncation
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIKQTYNTATSPLQLNLAAQTIQTKIEEKITAEKNTNWLRQTISAFVKTQSAWNSESEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQSVGNSEIITSVRYGKGALKATDTGDRTTRTSGVAVIEGNSPSLRLRSYDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATTKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVEVTRVDKYGHPVAGSQIKNTLYVVDGKSSGKDQQAKYGGAFLEELQAKYPELFARKQISTGVPMDPTVKIKQWSAKYFNGTNILGRGAGYVLKDQATNTYFSLAADNTFLPKSLVNPDHGTSS

SEQ ID NO: 53 - DNA, 2715 bp, artificial sequence
  T1 C-terminal truncation
gtgaacggtaaatattattattataaagaagatggaactcttcaaaagaattatgctttaaatattaatgggaaaactttcttctttgatgaaacaggagcattatcaaataatactttacctagtaaaaagggtaatatcactaataatgataacactaacagctttgctcaatataatcaggtctatagtacagatgctgcaaacttcgaacatgttgatcattatttgacagctgagagttggtatcgtcctaaatacatcttaaaagatggcaaaacatggacacagtcaacagaaaaagatttccgtcccttactgatgacatggtggcctgaccaagaaacgcagcgtcaatatgttaactacatgaatgcacagcttggtattcatcgaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaaccggaaagaaagatccaaggtatacagctgatcgcactatcggcggttacgaatttcttttggcaaacgatgtggataattctaatcctgtcgtgcaggccgaacaattgaactggctacattttctcatgaactttggtaacatttatgccaatgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagagcagaaatcaatcctaatgttgtcgggtattctttcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagcccataagaaccaagcataccgtccattattgttaactaccaacaatgggattaaagcatatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggggtcacagattttgaaatggcaccgcagtatgtgtcttcaacagatggttctttcttggattctgtgatccaaaacggctatgcttttacggaccgttatgatttaggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagataaaaaacaccctttatgtagttgatggtaagagttctggtaaagatcaacaagccaagtatgggggagctttcttagaggagctgcaagctaaatatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccaaatcacggaacaagcagttaa

SEQ ID NO: 54 - protein, 904 aa, artificial sequence
  T1 C-terminal truncation
VNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHRTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTNNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSS

SEQ ID NO: 55 - DNA, 2535 bp, artificial sequence
  T3 C-terminal truncation
agctttgctcaatataatcaggtctatagtacagatgctgcaaacttcgaacatgttgatcattatttgacagctgagagttggtatcgtcctaagtacatcttgaaggatggtaaaacatggacacagtcaacagaaaaagatttccgtcctttactgatgacatggtggcctgaccaagaaacgcagcgtcaatatgttaactacatgaatgcacagcttggtattcatcaaacatacaatacagcaaccagtccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagccgatcgcactatcggcggttacgaatttttgttagccaatgatgtggataattccaatcctgtcgtgcaggccgaacaattgaactggctacattttctcatgaactttggtaacatttatgccaatgatccggatgctaactttgattccattcgtgttgatgcggtagataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcattcgtgctcatgacagtgaagtgcaggacttgattcgcaatattattagagcagaaatcaatcctaatgttgtcgggtattcattcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagctcataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacggatggttctttcttggattctgtgatccaaaacggctatgcttttacggaccgttatgatttgggaatttccaaacctaataaatacgggacagccgatgatttggtgaaagcaataaaagcgttacacagcaagggtattaaggtaatggctgactgggtgcctgatcaaatgtatgcttttcctgaaaaagaagtggtaacagcaacccgcgttgataagtatgggactcctgttgcaggaagtcagatcaaaaacaccctttatgtagttgatggtaagagttctggtaaagatcaacaagccaagtatgggggagctttcttagaggagctgcaagcgaagtatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccataa

SEQ ID NO: 56 - protein, 844 aa, artificial sequence
  T3 C-terminal truncation
SFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNP

SEQ ID NO: 57 - DNA, 2535 bp, artificial sequence
  T3 C-terminal truncation
agctttgctcaatataatcaggtctatagtacagatgctgcaaacttcgaacatgttgatcattatttgacagccgaaagttggtatcgtcctaagtacatcttgaaggatggcaaaacatggacacagtcaacagaaaaagatttccgtcctttactgatgacatggtggcctgaccaagaaacgcagcgtcaatatgttaactacatgaatgcacagcttggtattcatcaaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaactgggaagaaggacccaaggtatacagctgataacactatcggcggttacgaatttcttttggcaaacgatgtggataattccaatcctgtcgtgcaggccgaacaattgaactggctccattttctcatgaactttggtaacatttatgccaatgatccggatgctaactttgattccattcgtgttgatgcggtagataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacccttaaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagaacagaaatcaatcctaatgttgtcgggtattctttcactatggaggaaatcaagaaggctttcgagatttacaacaaagacttgttagctacagagaagaaatacacacactataatacggcactttcttatgccctgcttttaaccaacaaatccagtgtgccgcgtgtctattatggggatatgtttacagatgacgggcaatacatggctcataagacgatcaattacgaagccatcgaaaccctgcttaaggctcgtattaagtatgtttcaggcggtcaagccatgcgcaatcaacaggttggcaattctgaaattattacgtctgtccgctatggtaaaggtgctttgaaagcaacggatacaggggaccgcaccacacgaacttcaggagtggccgtgattgaaggcaataacccttctttacgtttgaaggcttctgatcgtgttgttgtcaatatgggagcagcccataagaaccaagcataccgacctttactcttgaccacagataacggtatcaaggcttatcattccgatcaagaagcggctggtttggtgcgctacaccaatgacagaggggaattgatcttcacagcggctgatattaaaggctatgccaaccctcaagtttctggctatttaggtgtctgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggtgtcacagattttgaaatggcaccgcagtatgtgtcttcaacagatggttctttcttggattctgtgatccaaaacggctatgcttttacggatcgttatgatttgggaatttccaaacctaataaatacgggacagccgatgatttggttaaggccatcaaagcgttacacagcaagggcattaaggtaatggctgactgggtgcctgatcaaatgtatgctctccctgaaaaagaagtggtaacagcaacccgcgttgataagtatgggactcctgttgcaggaagtcagatcaaaaacaccctttatgtagttgatggtaagagttctggtaaagatcaacaagccaagtatgggggagccttcttagaggagctgcaagcgaagtatccggagctttttgcgagaaagcaaatttccacaggggttccgatggacccttcagttaagattaagcaatggtctgccaagtactttaatgggacaaatattttagggcgcggagcaggctatgtcttaaaagatcaggcaactaatacttacttcagtcttgtttcagacaacaccttccttcctaaatcgttagttaacccataa

SEQ ID NO: 58 - protein, 844 aa, artificial sequence
  T3 C-terminal truncation
SFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRTEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNP

SEQ ID NO: 59 - DNA, 2535 bp, artificial sequence
  T3 C-terminal truncation
agctttgctcaatataatcaggtctatagtacagatgctgcaaacttcgaacatgttgatcattatttgacagctgagagttggtatcgtcctaagtacatcttgaaagatggtaaaacatggacacagtcaacagaaaaagatttccgtcctttattgatgacatggtggcctgaccaagaaacacagcgtcaatatgtcaactacatgaatgcacagcttgggatcaagcaaacatacaatacagcaaccagtccgcttcaattaaatttagcggctcagacaatacaaactaagatcgaagaaaagatcactgcagaaaagaataccaattggctgcgtcagactatttcagcatttgttaagacacagtcagcttggaatagtgagagcgaaaaaccgtttgatgatcacttacaaaaaggggcattgctttacagtaacaatagcaagctaacttcacaggctaattccaactaccgtattttaaatcgcaccccgaccaatcaaaccggaaagaaagatccacggtatacagccgatcgcaccatcggtggttacgagttcttgctggctaatgatgtggataattccaatcctgttgttcaggccgaacagctgaactggctgcattttctcatgaactttggtaacatttatgccaacgatcctgatgctaactttgattccattcgtgttgatgcggtggacaatgtggatgctgacttacttcaaatcgctggtgattacctcaaagctgctaaagggattcataaaaatgataaggctgccaatgatcatttgtctattttagaggcatggagctataacgacactccttaccttcatgatgatggcgataatatgattaacatggacaatagattacgtctttccttgctttattcattagctaaacccttgaatcaacgttcaggcatgaatcctctcatcactaacagtctggtgaatcgaacagatgataacgctgaaactgccgcagtcccttcttattccttcattcgtgcccatgacagtgaagtgcaggatttgattcgcaatattattagagcagaaatcaatcctaatgttgttggttattctttcaccatggaggaaatcaagaaggctttcgagatttacaacaaagacttactggctacagagaagaaatacacacactataatacggcactttcttatgccctgcttttaactaacaaatccagtgtgccgcgtgtctattacggcgatatgttcacagatgacggtcagtacatggcacataagaccattaattacgaagccatcgaaactctgcttaaagcacggattaagtatgtttcaggcggtcaggccatgcgaaaccaaagtgttggcaattctgaaatcattacgtctgttcgctatggtaagggagccctgaaagcaacggatacaggagaccgcaccacacgcacttctggagtggccgtgattgaaggcaatagcccttctttacgtttgcgttcttatgatcgtgttgttgtcaatatgggagctgcccataagaaccaagcataccgacctttactcttgaccacagataacggtatcaaggcttatcattctgatcaagaagcggctggtttggtgcgctacaccaatgacagaggggaattgatcttcacagcggctgatatcaaaggctatgccaaccctcaagtttctggctatttaggtgtttgggtgccagtaggagctgcagctgatcaagatgtccgtgtggcagccagcactgccccatcaacagacggcaaatcagtgcatcaaaatgcagcccttgattctcgtgtcatgtttgaaggcttctcaaatttccaagcatttgcgactacaaaagaagagtatacgaatgtggtcattgctaagaatgtggataagtttgcggaatggggtgttacagactttgaaatggcaccgcaatatgtgtcttcaacagatggttctttcttggattctgtaattcaaaatggctatgcctttacggatcgttatgatctgggaatttccaaacctaataaatacgggacagccgatgatttggttaaggccatcaaagcattgcacagcaagggcattaaggttatggccgactgggtgcctgatcaaatgtatgctttccctgagaaagaagtggttgaagtcactcgtgtggacaaatatggacatcctgttgcaggcagtcaaatcaaaaacacactttatgtagttgatggtaagagttccggaaaggaccagcaggctaagtatgggggagctttcttagaagagctgcaagctaaatatccagagctctttgccagaaagcaaatttcaacaggggttccgatggacccaactgttaagattaagcaatggtctgccaagtactttaatggaacaaacattttagggcggggagcaggctatgtcttaaaggatcaggcaaccaatacttatttcagtcttgctgcagataataccttccttccgaaatcattagttaatccgtaa

SEQ ID NO: 60 - protein, 844 aa, artificial sequence
  T3 C-terminal truncation
SFAQYNQVYSTDAANFEHVDHYLTAESWYRPKYILKDGKTWTQSTEKDFRPLLMTWWPDQETQRQYVNYMNAQLGIKQTYNTATSPLQLNLAAQTIQTKIEEKITAEKNTNWLRQTISAFVKTQSAWNSESEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQSVGNSEIITSVRYGKGALKATDTGDRTTRTSGVAVIEGNSPSLRLRSYDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATTKEEYTNVVIAKNVDKFAEWGVTDFEMAPQYVSSTDGSFLDSVIQNGYAFTDRYDLGISKPNKYGTADDLVKAIKALHSKGIKVMADWVPDQMYAFPEKEVVEVTRVDKYGHPVAGSQIKNTLYVVDGKSSGKDQQAKYGGAFLEELQAKYPELFARKQISTGVPMDPTVKIKQWSAKYFNGTNILGRGAGYVLKDQATNTYFSLAADNTFLPKSLVNP

SEQ ID NO: 61 - DNA, 2535 bp, artificial sequence
  T3 C-terminal truncation
agctttgctcaatataatcaggtctatagtacagatgctgcaaacttcgaacatgttgatcattatttgacagctgagagttggtatcgtcctaaatacatcttaaaagatggcaaaacatggacacagtcaacagaaaaagatttccgtcccttactgatgacatggtggcctgaccaagaaacgcagcgtcaatatgttaactacatgaatgcacagcttggtattcatcgaacatacaatacagcaacttcaccgcttcaattgaatttagctgctcagacaatacaaactaagatcgaagaaaaaatcactgcagaaaagaataccaattggctgcgtcagactatttccgcatttgttaagacacagtcagcttggaacagtgacagcgaaaaaccgtttgatgatcacttacaaaaaggggcattgctttacagtaataatagcaaactaacttcacaggctaattccaactaccgtatcttaaatcgcaccccgaccaatcaaaccggaaagaaagatccaaggtatacagctgatcgcactatcggcggttacgaatttcttttggcaaacgatgtggataattctaatcctgtcgtgcaggccgaacaattgaactggctacattttctcatgaactttggtaacatttatgccaatgatccggatgctaactttgattccattcgtgttgatgcggtggataatgtggatgctgacttgctccaaattgctggggattacctcaaagctgctaaggggattcataaaaatgataaggctgctaatgatcatttgtctattttagaggcatggagttataatgatactccttaccttcatgatgatggcgacaatatgattaacatggataacaggttacgtctttccttgctttattcattagctaaacctttgaatcaacgttcaggcatgaatcctctgatcactaacagtttggtgaatcgaactgatgataatgctgaaactgccgcagtcccttcttattccttcatccgtgcccatgacagtgaagtgcaggacttgattcgcaatattattagagcagaaatcaatcctaatgttgtcgggtattctttcactatggaggaaatcaagaaggctttcgagatttacaacaaagacttattagctacagagaagaaatacacacactataatacggcactttcttatgccctgcttttaaccaacaaatccagtgtgccgcgtgtctattatggggatatgttcacagatgacgggcaatacatggctcataagacgatcaattacgaagccatcgaaacccttttaaaggctcgtattaagtatgtttcaggcggtcaagccatgcgcaatcaacaggttggcaattctgaaatcattacgtctgtccgctatggtaaaggtgctttgaaagcaacggatacaggggaccgcaccacacggacttcaggagtggccgtgattgaaggcaataacccttctttacgtttgaaggcttctgatcgcgtggttgtcaatatgggagcagcccataagaaccaagcataccgtccattattgttaactaccaacaatgggattaaagcatatcattccgatcaagaagcggctggtttggtgcgctacaccaatgacagaggggaattgatcttcacagcggctgatattaaaggctatgccaaccctcaagtttctggctatttaggtgtttgggttccagtaggcgctgccgctgatcaagatgttcgcgttgcggcttcaacggccccatcaacagatggcaagtctgtgcatcaaaatgcggcccttgattcacgcgtcatgtttgaaggtttctctaatttccaagcattcgccactaaaaaagaggaatataccaatgttgtgattgctaagaatgtggataagtttgcggaatggggggtcacagattttgaaatggcaccgcagtatgtgtcttcaacagatggttctttcttggattctgtgatccaaaacggctatgcttttacggaccgttatgatttaggaatttccaaacctaataaatacgggacagccgatgatttggtgaaagccatcaaagcgttacacagcaagggcattaaggtaatggctgactgggtgcctgatcaaatgtatgctctccctgaaaaagaagtggtaacagcaacccgtgttgataagtatgggactcctgttgcaggaagtcagataaaaaacaccctttatgtagttgatggtaagagttctggtaaagatcaacaagccaagtatgggggagctttcttagaggagctgcaagctaaatatccggagctttttgcgagaaaacaaatttccacaggggttccgatggacccttcagttaagattaagcaatggtctgccaagtactttaatgggacaaatattttagggcgcggagcaggctatgtcttaaaagatcaggcaaccaatacttacttcagtcttgtttcagacaacaccttccttcctaaatcgttagttaacccataa

SEQ ID NO: 62 - protein, 844 aa, artificial sequence
  T3 C-terminal truncation
SFAQYNQVYSTDAANFEHVDHYLTAESWYRPKYILKDGKTWTQSTEKDFRPLLMTWWPDQETQRQYVNYMNAQLGIHRTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADRTIGGYEFLLANDVDNSNPVVQAEQLNWLHFLMNFGNIYANDPDANFDSIRVDAVDNVDADLLQIAGDYLKAAKGIHKNDKAANDHLSILEAWSYNDTPYLHDDGDNMINMDNRLRLSLLYSLAKPLNQRSGMNPLITNSLVNRTDDNAETAAVPSYSFIRAHDSEVQDLIRNIIRAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTNNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNP

(19) United States
(12) Patent Application Publication
(10) Pub. No.: US 2017/0198323 A1
(43) Pub. Date: Jul. 13, 2017

(54) ENZYMATIC SYNTHESIS OF SOLUBLE GLUCAN FIBER

(71) Applicant: E I DU PONT DE NEMOURS AND COMPANY, Wilmington, DE (US)

(72) Inventors: Qiong Cheng, Wilmington, DE (US); Robert Dicosimo, Chadds Ford, PA (US); Arthur Ouwehand, Inga (FI); Zheng You, Wilmington, DE (US); Mark S. Payne, Wilmington, DE (US); Jahnavi Chandra Prasad, Wilmington, DE (US)

(21) Appl. No.: 15/313,285

(22) PCT Filed: May 22, 2015
(86) PCT No.: PCT/US15/32133

§ 371 Date: Nov. 22, 2016

Related U.S. Application Data
(60) Provisional application No. 62/004,308, filed on May 29, 2014.

(51) Int. Cl. C12P 19/18; A61K 31/702; A61K 8/60; A61Q 19/00; A23G 1/40; A23K 20/10; A23G 9/34; A23L 2/52; A23L 7/139; A23L 27/30; A23L 33/10; A23L 33/21; C07H 3/06; A23G 3/42

(52) U.S. Cl. CPC C12P 19/18; C07H 3/06; C12Y 301/01059; C12Y 302/01011; A61K 31/702; A61K 8/60; A61Q 19/00; A23G 1/40; A23G 3/42; A23G 9/34; A23L 2/52; A23L 7/139; A23L 27/30; A23L 33/10; A23L 33/21; A23K 20/10; A61K 2800/85; A23V 2002/00

(57) ABSTRACT

An enzymatically produced soluble α-glucan fiber composition is provided suitable for use as a digestion resistant fiber in food and feed applications. The soluble α-glucan fiber composition can be blended with one or more additional food ingredients to produce fiber-containing compositions. Methods for the production and use of compositions comprising the soluble α-glucan fiber are also provided.

### CROSS-REFERENCE TO RELATED APPLICATIONS

This application claims priority to and the benefit of U.S. provisional application No. 62/004,308, titled “Enzymatic Synthesis of Soluble Glucan Fiber,” filed May 29, 2014, the disclosure of which is incorporated by reference herein in its entirety.

### INCORPORATION BY REFERENCE OF THE SEQUENCE LISTING

The sequence listing provided in the file named “20150515_CL6056WOPCT_SequenceListing_ST25.txt” with a size of 433,860 bytes which was created on May 11, 2015 and which is filed herewith, is incorporated by reference herein in its entirety.

### FIELD OF THE INVENTION

This disclosure relates to a soluble α-glucan fiber, compositions comprising the soluble fiber, and methods of making and using the soluble α-glucan fiber. The soluble α-glucan fiber is highly resistant to digestion in the upper gastrointestinal tract, exhibits an acceptable rate of gas production in the lower gastrointestinal tract, is well tolerated as a dietary fiber, and has one or more beneficial properties typically associated with a soluble dietary fiber.

### BACKGROUND OF THE INVENTION

Dietary fiber (both soluble and insoluble) is a nutrient important for health, digestion, and preventing conditions such as heart disease, diabetes, obesity, diverticulitis, and constipation. However, most humans do not consume the daily recommended intake of dietary fiber. The 2010 Dietary Fiber Guidelines for Americans (U.S. Department of Agriculture and U.S. Department of Health and Human Services. Dietary Guidelines for Americans, 2010. 7th Edition, Washington, D.C.: U.S. Government Printing Office, December 2010) reports that the insufficiency of dietary fiber intake is a public health concern for both adults and children. As such, there remains a need to increase the amount of daily dietary fiber intake, especially soluble dietary fiber suitable for use in a variety of food applications.

Historically, dietary fiber was defined as the non-digestible carbohydrates and lignin that are intrinsic and intact in plants. This definition has been expanded to include carbohydrate polymers with three or more monomeric units that are not significantly hydrolyzed by the endogenous enzymes in the upper gastrointestinal tract of humans and which have a beneficial physiological effect demonstrated by generally accepted scientific evidence. Soluble oligosaccharide fiber products (such as oligomers of fructans, glucans, etc.) are currently used in a variety of food applications. However, many of the commercially available soluble fibers have undesirable properties such as low tolerance (causing undesirable effects such as abdominal bloating or gas, diarrhea, etc.), lack of digestion resistance, instability at low pH (e.g., pH 4 or less), high cost or a production process that requires at least one acid-catalyzed heat treatment step to randomly rearrange the more-digestible glycosidic bonds (for example, α-(1,4) linkages in glucans) into more highly-branched compounds with linkages that are more digestion-resistant. A process that uses only naturally occurring enzymes to synthesize suitable glucan fibers from a safe and readily-available substrate, such as sucrose, may be more attractive to consumers.

Various bacterial species have the ability to synthesize dextran oligomers from sucrose. Jeanes et al. (JACS (1954) 76:5041-5052) describe dextrans produced from 96 strains of bacteria. The dextrans were reported to contain a significant percentage (50-97%) of α-(1,6) glycosidic linkages with varying amounts of α-(1,3) and α-(1,4) glycosidic linkages. The enzymes present (both number and type) within the individual strains were not reported, and the dextran profiles in certain strains exhibited variability, where the dextrans produced by each bacterial species may be the product of more than one enzyme produced by each bacterial species.

Glucosyltransferases (glucansucrases; GTFs) belonging to glucoside hydrolase family 70 are able to polymerize the D-glucosyl units of sucrose to form homooligosaccharides or homopolysaccharides. Glucansucrases are further classified by the type of saccharide oligomer formed. For example, dextransucrases are those that produce saccharide oligomers with predominantly α-(1,6) glycosidic linkages (“dextrans”), and mutansucrases are those that tend to produce insoluble saccharide oligomers with a backbone rich in α-(1,3) glycosidic linkages. Mutansucrases are characterized by common amino acids. For example, A. Shimamura et al. (J. Bacteriology, (1994) 176:4845-4850) investigated the structure-function relationship of GTFs from Streptococcus mutans GS5, and identified several amino acid positions which influence the nature of the glucan product synthesized by GTFs where changes in the relative amounts of α-(1,3)- and α-(1,6)-anomeric linkages were produced. Reuteransucrases tend to produce saccharide oligomers rich in α-(1,4), α-(1,6), and α-(1,4,6) glycosidic linkages, and alternansucrases are those that tend to produce saccharide oligomers with a linear backbone comprised of alternating α-(1,3) and α-(1,6) glycosidic linkages. Some of these enzymes are capable of introducing other glycosidic linkages, often as branch points, to varying degrees. V. Monchois et al. (FEMS Microbiol Rev., (1999) 23:131-151) discusses the proposed mechanism of action and structure-function relationships for several glucansucrases. H. Leemhuis et al. (J. Biotechnol., (2013) 163:250-272) describe characteristic three-dimensional structures, reactions, mechanisms, and α-glucan analyses of glucansucrases.

A non-limiting list of patents and published patent applications describing the use of glucansucrases (wild type, truncated or variants thereof) to produce saccharide oligomers has been reported for dextran (U.S. Pat. Nos. 4,649,058 and 7,897,373; and U.S. Patent Appl. Pub. No. 2011-0178289A1), reuteran (U.S. Patent Application Publication No. 2009-0297663A1 and U.S. Pat. No. 6,867,026), alternan and/or maltoalternan oligomers (“MAOs”) (U.S. Pat. Nos. 7,402,420 and 7,524,645; U.S. Patent Appl. Pub. No. 2010-0122378A1; and European Patent EP1151085B1), α-(1,2) branched dextrans (U.S. Pat. No. 7,439,049), and a mixed-linkage saccharide oligomer (lacking an alternan-like backbone) comprising a mix of α-(1,3), α-(1,6), and α-(1,3,6) linkages (U.S. Patent Appl. Pub. No. 2005-0059633A1). U.S. Patent Appl. Pub. No. 2009-0300798A1 to Kol-Jakon et al. discloses genetically modified plant cells expressing a mutansucrase to produce modified starch.

Enzymatic production of isomaltose, isomaltooligosaccharides, and dextran using a combination of a glucosyltransferase and an α-glucanohydrolase has been reported. U.S. Pat. No. 2,776,925 describes a method for enzymatic production of dextran of intermediate molecular weight comprising the simultaneous action of dextransucrase and dextranase. U.S. Pat. No. 4,861,381A describes a method to enzymatically produce a composition comprising 39-80% isomaltose using a combination of a dextransucrase and a dextranase. Goulas et al. (Enz. Microb. Tech (2004) 35:327-338 describes batch synthesis of isomaltooligosaccharides (IMOs) from sucrose using a dextransucrase and a dextranase. U.S. Pat. No. 8,192,956 discloses a method to enzymatically produce isomaltooligosaccharides (IMOs) and low molecular weight dextran for clinical use using a recombinantly expressed hybrid gene comprising a gene encoding an α-glucanase and a gene encoding dextransucrase fused together; wherein the glucanase gene is a gene from Arthrobacter sp., wherein the dextransucrase gene is a gene from Leuconostoc sp.

Hayacibara et al. (Carb. Res. (2004) 339:2127-2137) describe the influence of mutanase and dextranase on the production and structure of glucans formed by glucosyltransferases from sucrose within dental plaque. The reported purpose of the study was to evaluate the production and the structure of glucans synthesized by GTFs in the presence of mutanase and dextranase, alone or in combination, in an attempt to elucidate some of the interactions that may occur during the formation of dental plaque. Mutanases (glucan endo-1,3-α-glucanohydrolases) are produced by some fungi, including Trichoderma, Aspergillus, Penicillium, and Cladosporium, and by some bacteria, including Streptomyces, Flavobacterium, Bacteroides, Bacillus, and Paenibacillus. W. Suyotha et al., (Biosci, Biotechnol. Biochem., (2013) 77:639-647) describe the domain structure and impact of domain deletions on the activity of an α-1,3-glucanohydrolases from Bacillus circulans KA-304. Y. Hakamada et al. (Biochimie, (2008) 90:525-533) describe the domain structure analysis of several mutanases, and a phylogenetic tree for mutanases is presented. I. Shimotsuura et al, (Appl. Environ. Microbiol., (2008) 74:2759-2765) report the biochemical and molecular characterization of mutanase from Paenibacillus sp. Strain RM1, where the N-terminal domain had strong mutan-binding activity but no mutanase activity, whereas the C-terminal domain was responsible for mutanase activity but had mutan-binding activity significantly lower than that of the intact protein. C. C. Fuglsang et al. (J. Biol. Chem., (2000) 275:2009-2018) describe the biochemical analysis of recombinant fungal mutanases (endoglucanases), where the fungal mutanases are comprised of a NH₂-terminal catalytic domain and a putative COOH-terminal polysaccharide binding domain.

Dextranases (α-1,6-glucan-6-glucanohydrolases) are enzymes that hydrolyzes α-1,6-linkages of dextran. N. Suzuki et al. (J. Biol. Chem. (2012) 287: 19916-19926) describes the crystal structure of Streptococcus mutans dextranase and identifies three structural domains, including domain A that contains the enzyme's catalytic module, and a dextran-binding domain C; the catalytic mechanism was also described relative to the enzyme structure. A. M. Larsson et al. (Structure, (2003) 11:1111-1121) reports the crystal structure of dextranase from Penicillium minioluteum, where the structure is used to define the reaction mechanism. H-K Kang et al. (Yeast, (2005) 22:1239-1248) describes the characterization of a dextranase from Lipomyces starkeyi. T. Igarashi et al. (Microbiol. Immunol., (2004) 48:155-162) describe the molecular characterization of dextranase from Streptococcus rattus, where the conserved region of the amino acid sequence contained two functional domains, catalytic and dextran-binding sites.

Various saccharide oligomer compositions have been reported in the art. For example, U.S. Pat. No. 6,486,314 discloses an α-glucan comprising at least 20, up to about 100,000 α-anhydroglucose units, 38-48% of which are 4-linked anhydroglucose units, 17-28% are 6-linked anhydroglucose units, and 7-20% are 4,6-linked anhydroglucose units and/or gluco-oligosaccharides containing at least two 4-linked anhydroglucose units, at least one 6-linked anhydroglucose unit and at least one 4,6-linked anhydroglucose unit. U.S. Patent Appl. Pub. No. 2010-0284972A1 discloses a composition for improving the health of a subject comprising an α-(1,2)-branched α-(1,6) oligodextran. U.S. Patent Appl. Pub. No. 2011-0020496A1 discloses a branched dextrin having a structure wherein glucose or isomaltooligosaccharide is linked to a non-reducing terminal of a dextrin through an α-(1,6) glycosidic bond and having a DE of 10 to 52. U.S. Pat. No. 6,630,586 discloses a branched maltodextrin composition comprising 22-35% (1,6) glycosidic linkages; a reducing sugars content of <20%; a polymolecularity index (Mp/Mn) of <5; and number average molecular weight (Mn) of 4500 g/mol or less. U.S. Pat. No. 7,612,198 discloses soluble, highly branched glucose polymers, having a reducing sugar content of less than 1%, a level of α-(1,6) glycosidic bonds of between 13 and 17% and a molecular weight having a value of between 0.9×10⁵ and 1.5×10⁵ daltons, wherein the soluble highly branched glucose polymers have a branched chain length distribution profile of 70 to 85% of a degree of polymerization (DP) of less than 15, of 10 to 14% of DP of between 15 and 25 and of 8 to 13% of DP greater than 25.

Saccharide oligomers and/or carbohydrate compositions comprising the oligomers have been described as suitable for use as a source of soluble fiber in food applications (U.S. Pat. No. 8,057,840 and U.S. Patent Appl. Pub. Nos. 2010-0047432A1 and 2011-0081474A1). U.S. Patent Appl. Pub. No. 2012-0034366A1 discloses low sugar, fiber-containing carbohydrate compositions which are reported to be suitable for use as substitutes for traditional corn syrups, high fructose corn syrups, and other sweeteners in food products.

There remains a need to develop new soluble α-glucan fiber compositions that are digestion resistant, exhibit a relatively low level and/or slow rate of gas formation in the lower gastrointestinal tract, are well-tolerated, have low viscosity, and are suitable for use in foods and other applications. Preferably the α-glucan fiber compositions can be enzymatically produced from sucrose using enzymes already associated with safe use in humans.

### SUMMARY OF THE INVENTION

A soluble α-glucan fiber composition is provided that is suitable for use in a variety of applications including, but not limited to, food applications, compositions to improve gastrointestinal health, and personal care compositions. The soluble fiber composition may be directly used as an ingredient in food or may be incorporated into carbohydrate compositions suitable for use in food applications.

A process for producing the soluble α-glucan fiber composition is also provided.

Methods of using the soluble fiber composition or carbohydrate compositions comprising the soluble fiber composition in food applications are also provided. In certain aspects, methods are provided for improving the health of a subject comprising administering the present soluble fiber composition to a subject in an amount effective to exert at least one health benefit typically associated with soluble dietary fiber such as altering the caloric content of food, decreasing the glycemic index of food, altering fecal weight and supporting bowel function, altering cholesterol metabolism, provide energy-yielding metabolites through colonic fermentation, and possibly providing prebiotic effects.

A soluble α-glucan fiber composition is provided comprising, on a dry solids basis, the following:

a. 10-30% α-(1,3) glycosidic linkages;

b. 65-87% α-(1,6) glycosidic linkages;

c. less than 5% α-(1,3,6) glycosidic linkages;

d. a weight average molecular weight of less than 5000 Daltons;

e. a viscosity of less than 0.25 Pascal second (Pa·s) at 12 wt % in water at 20° C.;

f. a dextrose equivalence (DE) in the range of 4 to 40; and

g. a digestibility of less than 12% as measured by the Association of Analytical Communities (AOAC) method 2009.01;

h. a solubility of at least 20% (w/w) in pH 7 water at 25° C.; and

i. a polydispersity index of less than 5.

In another embodiment, a method to produce a soluble α-glucan fiber composition is provided, the method comprising:

a. providing a set of reaction components comprising:
i. sucrose;
ii. at least one polypeptide having glucosyltransferase activity, said polypeptide comprising an amino acid sequence having at least 90% identity to a sequence selected from SEQ ID NOs: 1 and 3;
iii. at least one polypeptide having α-glucanohydrolase activity; and
iv. optionally one or more acceptors;

b. combining the set of reaction components under suitable aqueous reaction conditions whereby a product comprising a soluble α-glucan fiber composition is produced; and

c. optionally isolating the soluble α-glucan fiber composition from the product of step (b).

In another embodiment, a method to produce the soluble α-glucan fiber composition described above is provided, the method comprising:

a. providing a set of reaction components comprising:
i. sucrose;
ii. at least one polypeptide having glucosyltransferase activity and comprising an amino acid sequence having at least 90% sequence identity to a sequence selected from SEQ ID NOs: 13, 16, 17, 19, 28, 30, 32, 34, 36, 38, 40, 42, 44, 46, 48, 50, 52, 54, 56, 58, 60, and 62; and
iii. optionally one or more acceptors;

b. combining the set of reaction components under suitable aqueous reaction conditions to form a single reaction mixture, whereby a product mixture comprising glucose oligomers is formed;

c. optionally isolating the soluble α-glucan fiber composition described above from the product mixture comprising glucose oligomers; and

d. optionally concentrating the soluble α-glucan fiber composition.

In another embodiment, a method is provided to make a blended carbohydrate composition, the method comprising combining the soluble α-glucan fiber composition described above with: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

In another embodiment, a method is provided to make a food product, the method comprising mixing one or more edible food ingredients with the present soluble α-glucan fiber composition or the carbohydrate composition comprising the present soluble α-glucan fiber composition, or a combination thereof.

In another embodiment, a method is provided to reduce the glycemic index of a food or beverage, the method comprising incorporating into the food or beverage the present soluble α-glucan fiber composition.

In another embodiment, a method is provided for inhibiting the elevation of blood-sugar level in a mammal, the method comprising a step of administering the present soluble α-glucan fiber composition to the mammal.

In another embodiment, a method is provided for lowering lipids in a living body of a mammal, the method comprising a step of administering the present soluble α-glucan fiber composition to the mammal.

In another embodiment, a method is provided for treating constipation in a mammal, the method comprising a step of administering the present soluble α-glucan fiber composition to the mammal.

In another embodiment, a method to alter fatty acid production in the colon of a mammal is provided, the method comprising a step of administering the present soluble α-glucan fiber composition to the mammal; preferably wherein the short chain fatty acid production is increased, the branched chain fatty acid production is decreased, or both.

In another embodiment, a low cariogenicity composition comprising the present soluble α-glucan fiber composition and at least one polyol is provided.

In another embodiment, a composition is provided comprising 0.01 to 99 wt % (dry solids basis) of the present soluble α-glucan fiber composition: a synbiotic, a peptide, a peptide hydrolysate, a protein, a protein hydrolysate, a soy protein, a dairy protein, an amino acid, a polyol, a polyphenol, a vitamin, a mineral, an herbal, an herbal extract, a fatty acid, a polyunsaturated fatty acid (PUFAs), a phytosteroid, betaine, a carotenoid, a digestive enzyme, a probiotic organism or any combination thereof.

In another embodiment, a product produced by any of the methods described herein is also provided; preferably wherein the product is the present soluble α-glucan composition.

### BRIEF DESCRIPTION OF THE BIOLOGICAL SEQUENCES

The following sequences comply with 37 C.F.R. §§1.821-1.825 (“Requirements for patent applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NO: 1 is the amino acid sequence of the Streptococcus mutans NN2025 Gtf-B glucosyltransferase as found in GENBANK® gi: 290580544.

SEQ ID NO: 2 is the nucleic acid sequence encoding a truncated Streptococcus mutans NN2025 Gtf-B (GENBANK® gi: 290580544) glucosyltransferase.

SEQ ID NO: 3 is the amino acid sequence of the truncated Streptococcus mutans NN2025 Gtf-B glucosyltransferase (also referred to herein as the “0544 glucosyltransferase” or “GTF0544”).

SEQ ID NO: 4 is the amino acid sequence of the Paenibacillus humicus mutanase as found in GENBANK® gi: 257153264).

SEQ ID NO: 5 is the nucleic acid sequence encoding the Paenibacillus humicus mutanase (GENBANK® gi: 257153265 where GENBANK® gi: 257153264 is the corresponding polynucleotide sequence) used in for expression in E. coli BL21(DE3).

SEQ ID NO: 6 is the amino acid sequence of the mature Paenibacillus humicus mutanase (GENBANK® gi: 257153264; referred to herein as the “3264 mutanase” or “MUT3264”) used for expression in E. coli BL21(DE3).

SEQ ID NO: 7 is the amino acid sequence of the B. subtilis AprE signal peptide used in the expression vector that was coupled to various enzymes for expression in B. subtilis.

SEQ ID NO: 8 is the nucleic acid sequence encoding the Paenibacillus humicus mutanase used for expression in B. subtilis host BG6006.

SEQ ID NO: 9 is the amino acid sequence of the mature Paenibacillus humicus mutanase used for expression in B. subtilis host BG6006. As used herein, this mutanase may also be referred to herein as “MUT3264”.

SEQ ID NO: 10 is the nucleic acid sequence encoding the Penicillium marneffei ATCC® 18224™ mutanase.

SEQ ID NO: 11 is the amino acid sequence of the Penicillium marneffei ATCC® 18224™ mutanase (GENBANK® gi: 212533325; also referred to herein as the “3325 mutanase” or “MUT3325”).

SEQ ID NO: 12 is the polynucleotide sequence of plasmid pTrex3.

SEQ ID NO: 13 is the amino acid sequence of the Streptococcus mutans glucosyltransferase as provided in GENBANK® gi:3130088.

SEQ ID NO: 14 is the nucleic acid sequence encoding a truncated version of the Streptococcus mutans glucosyltransferase.

SEQ ID NO: 15 is the nucleic acid sequence of plasmid pMP69.

SEQ ID NO: 16 is the amino acid sequence of a truncated Streptococcus mutans glucosyltransferase referred to herein as “GTF0088”.

SEQ ID NO: 17 is the amino acid sequence of the Streptococcus mutans LJ23 glucosyltransferase as provided in GENBANK® gi:387786207 (also referred to as the “6207” glucosyltransferase or the “GTF6207”.

SEQ ID NO: 18 is the nucleic acid sequence encoding a truncated Streptococcus mutans LJ23 glucosyltransferase.

SEQ ID NO: 19 is the amino acid sequence of a truncated version of the Streptococcus mutans LJ23 glucosyltransferase, also referred to herein as “GTF6207”.

SEQ ID NO: 20 is a 1630 bp nucleic acid sequence used in Example 8.

SEQ ID NOs: 21-22 are primers.

SEQ ID NO: 23 is the nucleic acid sequence of plasmid p6207-1. SEQ ID NO: 24 is a polynucleotide sequence of a terminator sequence.

SEQ ID NO: 25 is a polynucleotide sequence of a linker sequence.

SEQ ID NO: 26 is the native nucleotide sequence of GTF0088.

SEQ ID NO: 27 is the native nucleotide sequence of GTF5330.

SEQ ID NO: 28 is the amino acid sequence encoded by SEQ ID NO: 27.

SEQ ID NO: 29 is the native nucleotide sequence of GTF5318.

SEQ ID NO: 30 is the amino acid sequence encoded by SEQ ID NO: 29.

SEQ ID NO: 31 is the native nucleotide sequence of GTF5326.

SEQ ID NO: 32 is the amino acid sequence encoded by SEQ ID NO: 31.

SEQ ID NO: 33 is the native nucleotide sequence of GTF5312.

SEQ ID NO: 34 is the amino acid sequence encoded by SEQ ID NO: 33.

SEQ ID NO: 35 is the native nucleotide sequence of GTF5334.

SEQ ID NO: 36 is the amino acid sequence encoded by SEQ ID NO: 35.

SEQ ID NO: 37 is the native nucleotide sequence of GTF0095.

SEQ ID NO: 38 is the amino acid sequence encoded by SEQ ID NO: 37.

SEQ ID NO: 39 is the native nucleotide sequence of GTF0074.

SEQ ID NO: 40 is the amino acid sequence encoded by SEQ ID NO: 39.

SEQ ID NO: 41 is the native nucleotide sequence of GTF5320.

SEQ ID NO: 42 is the amino acid sequence encode by SEQ ID NO:

41.

SEQ ID NO: 43 is the native nucleotide sequence of GTF0081.

SEQ ID NO: 44 is the amino acid sequence encoded by SEQ ID NO: 43.

SEQ ID NO: 45 is the native nucleotide sequence of GTF5328.

SEQ ID NO: 46 is the amino acid sequence encoded by SEQ ID NO: 45.

SEQ ID NO: 47 is the nucleotide sequence of a T1 C-terminal truncation of GTF0088.

SEQ ID NO: 48 is the amino acid sequence encoded by SEQ ID NO: 47.

SEQ ID NO: 49 is the nucleotide sequence of a T1 C-terminal truncation of GTF5318.

SEQ ID NO: 50 is the amino acid sequence encoded by SEQ ID NO: 49.

SEQ ID NO: 51 is the nucleotide sequence of a T1 C-terminal truncation of GTF5328.

SEQ ID NO: 52 is the amino acid sequence encoded by SEQ ID NO: 51.

SEQ ID NO: 53 is the nucleotide sequence of a T1 C-terminal truncation of GTF5330.

SEQ ID NO: 54 is the amino acid sequence encoded by SEQ ID NO: 53.

SEQ ID NO: 55 is the nucleotide sequence of a T3 C-terminal truncation of GTF0088.

SEQ ID NO: 56 is the amino acid sequence encoded by SEQ ID NO: 55.

SEQ ID NO: 57 is the nucleotide sequence of a T3 C-terminal truncation of GTF5318.

SEQ ID NO: 58 is the amino acid sequence encoded by SEQ ID NO: 57.

SEQ ID NO: 59 is the nucleotide sequence of a T3 C-terminal truncation of GTF5328.

SEQ ID NO: 60 is the amino acid sequence encoded by SEQ ID NO: 59.

SEQ ID NO: 61 is the nucleotide sequence of a T3 C-terminal truncation of GTF5330.

SEQ ID NO: 62 is the amino acid sequence encoded by SEQ ID NO: 61.

### DETAILED DESCRIPTION OF THE INVENTION

In this disclosure, a number of terms and abbreviations are used. The following definitions apply unless specifically stated otherwise.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, the term “obtainable from” shall mean that the source material (for example, sucrose) is capable of being obtained from a specified source, but is not necessarily limited to that specified source.

As used herein, the term “effective amount” will refer to the amount of the substance used or administered that is suitable to achieve the desired effect. The effective amount of material may vary depending upon the application. One of skill in the art will typically be able to determine an effective amount for a particular application or subject without undo experimentation.

As used herein, the term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

As used herein, the terms “very slow to no digestibility”, “little or no digestibility”, and “low to no digestibility” will refer to the relative level of digestibility of the soluble glucan fiber as measured by the Association of Official Analytical Chemists International (AOAC) method 2009.01 (“AOAC 2009.01”; McCleary et al. (2010) J. AOAC Int., 93(1), 221-233); where little or no digestibility will mean less than 12% of the soluble glucan fiber composition is digestible, preferably less than 5% digestible, more preferably less than 1% digestible on a dry solids basis (d.s.b.). In another aspect, the relative level of digestibility may be alternatively be determined using AOAC 2011.25 (Integrated Total Dietary Fiber Assay) (McCleary et al., (2012) J. AOAC Int., 95 (3), 824-844.

As used herein, term “water soluble” will refer to the present glucan fiber composition comprised of fibers that are soluble at 20 wt % or higher in pH 7 water at 25° C.

As used herein, the terms “soluble fiber”, “soluble glucan fiber”, “α-glucan fiber”, “cane sugar fiber”, “glucose fiber”, “beet sugar fiber”, “soluble dietary fiber”, and “soluble glucan fiber composition” refer to the present fiber composition comprised of water soluble glucose oligomers having a glucose polymerization degree of 3 or more that is digestion resistant (i.e., exhibits very slow to no digestibility) with little or no absorption in the human small intestine and is at least partially fermentable in the lower gasterointestinal tract. Digestibility of the soluble glucan fiber composition is measured using AOAC method 2009.01. The present soluble glucan fiber composition is enzymatically synthesized from sucrose (α-D-Glucopyranosyl β-D-fructofuranoside; CAS#57-50-1) obtainable from, for example, sugarcane and/or sugar beets. In one embodiment, the present soluble α-glucan fiber composition is not alternan or maltoalternan oligosaccharide.

As used herein, “weight average molecular weight” or “M_{w}” is calculated as

M_{w}=ΣNᵢMᵢ²/ΣNᵢMᵢ;

where Mᵢ is the molecular weight of a chain and Nᵢ is the number of chains of that molecular weight. The weight average molecular weight can be determined by technics such as static light scattering, small angle neutron scattering, X-ray scattering, and sedimentation velocity.

As used herein, “number average molecular weight” or “Mₙ” refers to the statistical average molecular weight of all the polymer chains in a sample. The number average molecular weight is calculated as Mₙ=ΣNᵢMᵢ/ΣN₁ where Mᵢ is the molecular weight of a chain and Nᵢ is the number of chains of that molecular weight. The number average molecular weight of a polymer can be determined by technics such as gel permeation chromatography, viscometry via the (Mark-Houwink equation), and colligative methods such as vapor pressure osmometry, end-group determination or proton NMR.

As used herein, “polydispersity index”, “PDI”, “heterogeneity index”, and “dispersity” refer to a measure of the distribution of molecular mass in a given polymer (such as a glucose oligomer) sample and can be calculated by dividing the weight average molecular weight by the number average molecular weight (PDI=M_{w}/Mₙ).

It shall be noted that the terms “glucose” and “glucopyranose” as used herein are considered as synonyms and used interchangeably. Similarly the terms “glucosyl” and “glucopyranosyl” units are used herein are considered as synonyms and used interchangeably.

As used herein, “glycosidic linkages” or “glycosidic bonds” will refer to the covalent the bonds connecting the sugar monomers within a saccharide oligomer (oligosaccharides and/or polysaccharides). Example of glycosidic linkage may include α-linked glucose oligomers with 1,6-α-D-glycosidic linkages (herein also referred to as α-D-(1,6) linkages or simply “α-(1,6)” linkages); 1,3-α-D-glycosidic linkages (herein also referred to as α-D-(1,3) linkages or simply “α-(1,3)” linkages; 1,4-α-D-glycosidic linkages (herein also referred to as α-D-(1,4) linkages or simply “α-(1,4)” linkages; 1,2-α-D-glycosidic linkages (herein also referred to as α-D-(1,2) linkages or simply “α-(1,2)” linkages; and combinations of such linkages typically associated with branched saccharide oligomers.

As used herein, the terms “glucansucrase”, “glucosyltransferase”, “glucoside hydrolase type 70”, “GTF”, and “GS” will refer to transglucosidases classified into family 70 of the glycoside-hydrolases typically found in lactic acid bacteria such as Streptococcus, Leuconostoc, Weise/la or Lactobacillus genera (see Carbohydrate Active Enzymes database; “CAZy”; Cantarel et al., (2009) Nucleic Acids Res 37:D233-238). The GTF enzymes are able to polymerize the D-glucosyl units of sucrose to form homooligosaccharides or homopolysaccharides. Glucosyltransferases can be identified by characteristic structural features such as those described in Leemhuis et al. (J. Biotechnology (2013) 162:250-272) and Monchois et al. (FEMS Micro. Revs. (1999) 23:131-151). Depending upon the specificity of the GTF enzyme, linear and/or branched glucans comprising various glycosidic linkages may be formed such as α-(1,2), α-(1,3), α-(1,4) and α-(1,6). Glucosyltransferases may also transfer the D-glucosyl units onto hydroxyl acceptor groups. A non-limiting list of acceptors include carbohydrates, alcohols, polyols and flavonoids. Specific acceptors may also include maltose, isomaltose, isomaltotriose, and methyl-α-D-glucan. The structure of the resultant glucosylated product is dependent upon the enzyme specificity. A non-limiting list of glucosyltransferase sequences is provided as amino acid SEQ ID NOs: 1, 3, 13, 16, 17, 19, 28, 30, 32, 34, 36, 38, 40, 42, 44, 46, 48, 50, 52, 54, 56, 58, 60, and 62. In one aspect, the glucosyltransferase is expressed in a truncated and/or mature form. In another embodiment, the polypeptide having glucosyltransferase activity comprises an amino acid sequence having at least 90% identity, preferably 91, 92, 93, 94, 95, 96, 97, 98, 99 or 100% identity to SEQ ID NO: 1, 3, 13, 16, 17, 19, 28, 30, 32, 34, 36, 38, 40, 42, 44, 46, 48, 50, 52, 54, 56, 58, 60, or 62.

As used herein, the term “isomaltooligosaccharide” or “IMO” refers to a glucose oligomers comprised essentially of α-D-(1,6) glycosidic linkage typically having an average size of DP 2 to 20. Isomaltooligosaccharides can be produced commercially from an enzymatic reaction of α-amylase, pullulanase, β-amylase, and α-glucosidase upon corn starch or starch derivative products. Commercially available products comprise a mixture of isomaltooligosaccharides (DP ranging from 3 to 8, e.g., isomaltotriose, isomaltotetraose, isomaltopentaose, isomaltohexaose, isomaltoheptaose, isomaltooctaose) and may also include panose.

As used herein, the term “dextran” refers to water soluble α-glucans comprising at least 95% α-D-(1,6) glycosidic linkages (typically with up to 5% α-D-(1,3) glycosidic linkages at branching points) that are more than 10% digestible as measured by the Association of Official Analytical Chemists International (AOAC) method 2009.01 (“AOAC 2009.01”). Dextrans often have an average molecular weight above 1000 kDa. As used herein, enzymes capable of synthesizing dextran from sucrose may be described as “dextransucrases” (EC 2.4.1.5).

As used herein, the term “mutan” refers to water insoluble α-glucans comprised primarily (50% or more of the glycosidic linkages present) of 1,3-α-D glycosidic linkages and typically have a degree of polymerization (DP) that is often greater than 9. Enzymes capable of synthesizing mutan or α-glucan oligomers comprising greater than 50% 1,3-α-D glycosidic linkages from sucrose may be described as “mutansucrases” (EC 2.4.1.-) with the proviso that the enzyme does not produce alternan.

As used herein, the term “alternan” refers to α-glucans having alternating 1,3-α-D glycosidic linkages and 1,6-α-D glycosidic linkages over at least 50% of the linear oligosaccharide backbone. Enzymes capable of synthesizing alternan from sucrose may be described as “alternansucrases” (EC 2.4.1.140).

As used herein, the term “reuteran” refers to soluble α-glucan comprised 1,4-α-D-glycosidic linkages (typically >50%); 1,6-α-D-glycosidic linkages; and 4,6-disubstituted α-glucosyl units at the branching points. Enzymes capable of synthesizing reuteran from sucrose may be described as “reuteransucrases” (EC 2.4.1.-).

As used herein, the terms “α-glucanohydrolase” and “glucanohydrolase” will refer to an enzyme capable of hydrolyzing an α-glucan oligomer. As used herein, the glucanohydrolase may be defined by the endohydrolysis activity towards certain α-D-glycosidic linkages. Examples may include, but are not limited to, dextranases (EC 3.2.1.1; capable of endohydrolyzing α-(1,6)-linked glycosidic bonds), mutanases (EC 3.2.1.59; capable of endohydrolyzing α-(1,3)-linked glycosidic bonds), and alternanases (EC 3.2.1.-; capable of endohydrolytically cleaving alternan). Various factors including, but not limited to, level of branching, the type of branching, and the relative branch length within certain α-glucans may adversely impact the ability of an α-glucanohydrolase to endohydrolyze some glycosidic linkages.

As used herein, the term “dextranase” (α-1,6-glucan-6-glucanohydrolase; EC 3.2.1.11) refers to an enzyme capable of endohydrolysis of 1,6-α-D-glycosidic linkages (the linkage predominantly found in dextran). Dextranases are known to be useful for a number of applications including the use as ingredient in dentifrice for prevent dental caries, plaque and/or tartar and for hydrolysis of raw sugar juice or syrup of sugar canes and sugar beets. Several microorganisms are known to be capable of producing dextranases, among them fungi of the genera Penicillium, Paecilomyces, Aspergillus, Fusarium, Spicaria, Verticillium, Helminthosporium and Chaetomium; bacteria of the genera Lactobacillus, Streptococcus, Cellvibrio, Cytophaga, Brevibacterium, Pseudomonas, Corynebacterium, Arthrobacter and Flavobacterium, and yeasts such as Lipomyces starkeyi. Food grade dextranases are commercially available. An example of a food grade dextrinase is DEXTRANASE® Plus L, an enzyme from Chaetomium erraticum sold by Novozymes A/S, Bagsvaerd, Denmark.

As used herein, the term “mutanase” (glucan endo-1,3-α-glucosidase; EC 3.2.1.59) refers to an enzyme which hydrolytically cleaves 1,3-α-D-glycosidic linkages (the linkage predominantly found in mutan). Mutanases are available from a variety of bacterial and fungal sources. A non-limiting list of mutanases is provided as amino acid sequences 4, 6, 9, and 11. In one embodiment, a polypeptide having mutanase activity comprises an amino acid sequence having at least 90% identity, preferably at least 91, 92, 93, 94, 95, 96, 97, 98, 99 or 100% identity to SEQ ID NO: 4, 6, 9 or 11.

As used herein, the term “alternanase” (EC 3.2.1.-) refers to an enzyme which endo-hydrolytically cleaves alternan (U.S. Pat. No. 5,786,196 to Cote et al.).

As used herein, the term “wild type enzyme” will refer to an enzyme (full length and active truncated forms thereof) comprising the amino acid sequence as found in the organism from which was obtained and/or annotated. The enzyme (full length or catalytically active truncation thereof) may be recombinantly produced in a microbial host cell. The enzyme is typically purified prior to being used as a processing aid in the production of the present soluble α-glucan fiber composition. In one aspect, a combination of at least two wild type enzymes simultaneously present in the reaction system are used in order to obtain the present soluble glucan fiber composition. In one embodiment, the combination of at least two enzymes concomitantly present comprises at least one polypeptide having glucosyltransferase activity comprising an amino acid sequence having at least 90% amino acid sequence identity to SEQ ID NO: 1 or 3 and at least one polypeptide having mutanase activity comprising an amino acid sequence having at least 90% amino acid sequence identity to SEQ ID NO: 4, 6, 9 or 11. In a preferred embodiment, the combination of at least two enzymes concomitantly present comprises at least one polypeptide having glucosyltransferase activity comprising an amino acid sequence having at least 90%, preferably at least 91, 92, 93, 94, 95, 96, 97, 98, 99 or 100% amino acid sequence identity to SEQ ID NO: 1 or 3 and at least one polypeptide having mutanase activity comprising an amino acid sequence having at least 90%, preferably at least 91, 92, 93, 94, 95, 96, 97, 98, 99 or 100% amino acid sequence identity to SEQ ID NO: 4 or 6.

As used herein, the terms “substrate” and “suitable substrate” will refer to a composition comprising sucrose. In one embodiment, the substrate composition further comprises one or more suitable acceptors, such as maltose, isomaltose, isomaltotriose, and methyl-α-D-glucan. In one embodiment, a combination of at least one glucosyltransferase capable of forming glucose oligomers is used in combination with at least one α-glucanohydrolase in the same reaction mixture (i.e., they are simultaneously present and active in the reaction mixture). As such the “substrate” for the α-glucanohydrolase (when present) are the glucose oligomers concomitantly being synthesized in the reaction mixture by the glucosyltransferase from sucrose. In one embodiment, a two-enzyme method (i.e., at least one glucosyltransferase (GTF) and at least one α-glucanohydrolase) where the enzymes are not used concomitantly in the reaction mixture is excluded, by proviso, from the methods disclosed herein.

As used herein, the terms “suitable enzymatic reaction mixture”, “suitable reaction components”, “suitable aqueous reaction mixture”, and “reaction mixture”, refer to the materials (suitable substrate(s)) and water in which the reactants come into contact with the enzyme(s). The suitable reaction components may be comprised of a plurality of enzymes. In one aspect, the suitable reaction components comprise at least one glucansucrase enzyme. In a further aspect, the suitable reaction components comprise at least one glucansucrase and at least one α-glucanohydrolase; preferably at least one polypeptide having mutanase activity.

As used herein, “one unit of glucansucrase activity” or “one unit of glucosyltransferase activity” is defined as the amount of enzyme required to convert 1 μmol of sucrose per minute when incubated with 200 g/L sucrose at pH 5.5 and 37° C. The sucrose concentration was determined using HPLC.

As used herein, “one unit of dextranase activity” is defined as the amount of enzyme that forms 1 μmol reducing sugar per minute when incubated with 0.5 mg/mL dextran substrate at pH 5.5 and 37° C. The reducing sugars were determined using the PAHBAH assay (Lever M., (1972), A New Reaction for Colorimetric Determination of Carbohydrates, Anal. Biochem. 47, 273-279).

As used herein, “one unit of mutanase activity” is defined as the amount of enzyme that forms 1 μmol reducing sugar per minute when incubated with 0.5 mg/mL mutan substrate at pH 5.5 and 37° C. The reducing sugars were determined using the PAHBAH assay (Lever M., supra).

As used herein, the term “enzyme catalyst” refers to a catalyst comprising an enzyme or combination of enzymes having the necessary activity to obtain the desired soluble glucan fiber composition. In certain embodiments, a combination of enzyme catalysts may be required to obtain the desired soluble glucan fiber composition. The enzyme catalyst(s) may be in the form of a whole microbial cell, permeabilized microbial cell(s), one or more cell components of a microbial cell extract(s), partially purified enzyme(s) or purified enzyme(s). In certain embodiments the enzyme catalyst(s) may also be chemically modified (such as by pegylation or by reaction with cross-linking reagents). The enzyme catalyst(s) may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997.

As used herein, “pharmaceutically-acceptable” means that the compounds or compositions in question are suitable for use in contact with the tissues of humans and other animals without undue toxicity, incompatibility, instability, irritation, allergic response, and the like, commensurate with a reasonable benefit/risk ratio.

As used herein, the term “oligosaccharide” refers to homopolymers containing between 3 and about 30 monosaccharide units linked by α-glycosidic bonds.

As used herein the term “polysaccharide” refers to homopolymers containing greater than 30 monosaccharide units linked by α-glycosidic bonds.

As used herein, the term “food” is used in a broad sense herein to include a variety of substances that can be ingested by humans including, but not limited to, beverages, dairy products, baked goods, energy bars, jellies, jams, cereals, dietary supplements, and medicinal capsules or tablets.

As used herein, the term “pet food” or “animal feed” is used in a broad sense herein to include a variety of substances that can be ingested by nonhuman animals and may include, for example, dog food, cat food, and feed for livestock.

A “subject” is generally a human, although as will be appreciated by those skilled in the art, the subject may be a non-human animal. Thus, other subjects may include mammals, such as rodents (including mice, rats, hamsters and guinea pigs), cats, dogs, rabbits, cows, horses, goats, sheep, pigs, and primates (including monkeys, chimpanzees, orangutans and gorillas).

The term “cholesterol-related diseases”, as used herein, includes but is not limited to conditions which involve elevated levels of cholesterol, in particular non-high density lipid (non-HDL) cholesterol in plasma, e.g., elevated levels of LDL cholesterol and elevated HDL/LDL ratio, hypercholesterolemia, and hypertriglyceridemia, among others. In patients with hypercholesteremia, lowering of LDL cholesterol is among the primary targets of therapy. In patients with hypertriglyceridemia, lower high serum triglyceride concentrations are among the primary targets of therapy. In particular, the treatment of cholesterol-related diseases as defined herein comprises the control of blood cholesterol levels, blood triglyceride levels, blood lipoprotein levels, blood glucose, and insulin sensitivity by administering the present glucan fiber or a composition comprising the present glucan fiber.

As used herein, “personal care products” means products used in the cosmetic treatment hair, skin, scalp, and teeth, including, but not limited to shampoos, body lotions, shower gels, topical moisturizers, toothpaste, tooth gels, mouthwashes, mouthrinses, anti-plaque rinses, and/or other topical treatments. In some particularly preferred embodiments, these products are utilized on humans, while in other embodiments, these products find cosmetic use with non-human animals (e.g., in certain veterinary applications).

As used herein, the terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, may not affect the functional properties of the encoded protein. For example, any particular amino acid in an amino acid sequence disclosed herein may be substituted for another functionally equivalent amino acid. For the purposes of the present invention, substitutions are defined as exchanges within one of the following five groups:
1. Small aliphatic, nonpolar or slightly polar residues: Ala, Ser, Thr (Pro, Gly);
2. Polar, negatively charged residues and their amides: Asp, Asn,
Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, and Trp.
   Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

As used herein, the term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

As used herein, “synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as pertaining to a DNA sequence, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequences to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

As used herein, “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may include regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein, “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

As used herein, the term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

As used herein, the term “expression” refers to the transcription and stable accumulation of sense (mRNA) or antisense RNA derived from the nucleic acid molecule of the invention. Expression may also refer to translation of mRNA into a polypeptide.

As used herein, “transformation” refers to the transfer of a nucleic acid molecule into the genome of a host organism, resulting in genetically stable inheritance. In the present invention, the host cell's genome includes chromosomal and extrachromosomal (e.g., plasmid) genes. Host organisms containing the transformed nucleic acid molecules are referred to as “transgenic”, “recombinant” or “transformed” organisms.

As used herein, the term “sequence analysis software” refers to any computer algorithm or software program that is useful for the analysis of nucleotide or amino acid sequences. “Sequence analysis software” may be commercially available or independently developed. Typical sequence analysis software will include, but is not limited to, the GCG suite of programs (Wisconsin Package Version 9.0, Accelrys Software Corp., San Diego, Calif.), BLASTP, BLASTN, BLASTX (Altschul et al., J. Mol. Biol. 215:403-410 (1990)), and DNASTAR (DNASTAR, Inc. 1228 S. Park St. Madison, Wis. 53715 USA), CLUSTALW (for example, version 1.83; Thompson et al., Nucleic Acids Research, 22(22):4673-4680 (1994)), and the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Publisher: Plenum, New York, N.Y.), Vector NTI (Informax, Bethesda, Md.) and Sequencher v. 4.05. Within the context of this application it will be understood that where sequence analysis software is used for analysis, that the results of the analysis will be based on the “default values” of the program referenced, unless otherwise specified. As used herein “default values” will mean any set of values or parameters set by the software manufacturer that originally load with the software when first initialized.

### Structural and Functional Properties of the Soluble α-Glucan Fiber Composition Disclosed Herein

Human gastrointestinal enzymes readily recognize and digest linear α-glucan oligomers having a substantial amount of α-(1,4) glycosidic bonds. Replacing these linkages with alternative linkages such as α-(1,2), α-(1,3), and α-(1,6) typically reduces the digestibility of the α-glucan oligomers. Increasing the degree of branching (using alternative linkages) may also reduce the relative level of digestibility.

The present soluble α-glucan fiber composition was prepared from cane sugar (sucrose) using one or more enzymatic processing aids that have essentially the same amino acid sequences as found in nature (or catalytically active truncations thereof) from microorganisms which having a long history of exposure to humans (microorganisms naturally found in the oral cavity or found in foods such a beer, fermented soybeans, etc.) and/or enzymes generally recognized as safe (GRAS). The soluble fibers have slow to no digestibility, exhibit high tolerance (i.e., as measured by an acceptable amount of gas formation), low viscosity (enabling use in a broad range of food applications), and are at least partially fermentable by gut microflora, providing possible prebiotic effects (for example, increasing the number and/or activity of bifidobacteria and lactic acid bacteria reported to be associated with providing potential prebiotic effects).

The soluble α-glucan fiber composition disclosed herein is characterized by the following combination of parameters:
a. 10% to 30% α-(1,3) glycosidic linkages;
b. 65% to 87% α-(1,6) glycosidic linkages;
c. less than 5% α-(1,3,6) glycosidic linkages;
d. a weight average molecular weight (Mw) of less than 5000 Daltons;
e. a viscosity of less than 0.25 Pascal second (Pa·s) at 12 wt % in water 20° C.;
f. a dextrose equivalence (DE) in the range of 4 to 40, preferably 10 to 40; and
g. a digestibility of less than 12% as measured by the Association of Analytical Communities (AOAC) method 2009.01;
h. a solubility of at least 20% (w/w) in pH 7 water at 25° C.; and
i. a polydispersity index (PDI) of less than 5.

The soluble α-glucan fiber composition disclosed herein comprises 10-30%, preferably 10-25%, α-(1,3) glycosidic linkages.

In certain embodiments, in addition to the α-(1,3) glycosidic linkage embodiments described above, the present soluble α-glucan fiber composition further comprises 65-87%, preferably 70-85%, more preferably 75-82% α-(1,6) glycosidic linkages.

In certain embodiments, in addition to the α-(1,3) and α-(1,6) glycosidic linkage content described above, the soluble α-glucan fiber composition further comprises less than 5%, preferably less than 4%, 3%, 2% or 1% α-(1,3,6) glycosidic linkages.

In certain embodiments, in addition to the above mentioned glycosidic linkage content, the soluble α-glucan fiber composition further comprises less than 5%, preferably less than 1%, and most preferably less than 0.5% α-(1,4) glycosidic linkages.

In another embodiment, in addition to the above mentioned glycosidic linkage amounts, the α-glucan fiber composition comprises a weight average molecular weight (M_{w}) of less than 5000 Daltons, preferably less than 2500 Daltons, more preferably between 500 and 2500 Daltons, and most preferably about 500 to about 2000 Daltons.

In another embodiment, in addition to any combination of the above features, the α-glucan fiber composition comprises a viscosity of less than 250 centipoise (cP) (0.25 Pascal second (Pas), preferably less than 10 centipoise (cP) (0.01 Pascal second (Pas)), preferably less than 7 cP (0.007 Pas), more preferably less than 5 cP (0.005 Pas), more preferably less than 4 cP (0.004 Pas), and most preferably less than 3 cP (0.003 Pas) at 12 wt % in water at 20° C.

The soluble α-glucan composition has a digestibility of less than 10%, preferably less than 9%, 8%, 7%, 6%, 5%, 4%, 3%, 2% or 1% digestible as measured by the Association of Analytical Communities (AOAC) method 2009.01. In another aspect, the relative level of digestibility may be alternatively determined using AOAC 2011.25 (Integrated Total Dietary Fiber Assay) (McCleary et al., (2012) J. AOAC Int., 95 (3), 824-844.

In addition to any of the above embodiments, in certain embodiments, the soluble α-glucan fiber composition has a solubility of at least 20% (w/w), preferably at least 30%, 40%, 50%, 60%, or 70% in pH 7 water at 25° C.

In certain embodiments, the soluble α-glucan fiber composition comprises a reducing sugar content of less than 10 wt %, preferably less than 5 wt %, and most preferably 1 wt % or less.

In certain embodiments, the soluble α-glucan fiber composition comprises a number average molecular weight (Mn) between 400 and 2000 g/mole; preferably 500 to 1500 g/mole.

In certain embodiments, the soluble α-glucan fiber composition comprises a caloric content of less than 4 kcal/g, preferably less than 3 kcal/g, more preferably less than 2.5 kcal/g, and most preferably about 2 kcal/g or less.

### Compositions Comprising Glucan Fibers

Depending upon the desired application, the soluble α-glucan fibers/fiber composition may be formulated (e.g., blended, mixed, incorporated into, etc.) with one or more other materials suitable for use in foods, personal care products and/or pharmaceuticals. As such, the present disclosure includes compositions comprising the soluble α-glucan fiber composition. The term “compositions comprising the soluble α-glucan fiber composition” in this context may include, for example, a nutritional or food composition, such as food products, food supplements, dietary supplements (for example, in the form of powders, liquids, gels, capsules, sachets or tables) or functional foods. In certain embodiments, “compositions comprising the soluble α-glucan fiber composition” includes personal care products, cosmetics, and pharmaceuticals.

The soluble α-glucan fibers/fiber composition may be directly included as an ingredient in a desired product (e.g., foods, personal care products, etc.) or may be blended with one or more additional food grade materials to form a carbohydrate composition that is used in the desired product (e.g., foods, personal care products, etc.). The amount of the soluble α-glucan fiber composition incorporated into the carbohydrate composition may vary according to the application. As such, the present invention comprises a carbohydrate composition comprising the soluble α-glucan fiber composition. In certain embodiments, the carbohydrate composition comprises 0.01 to 99 wt % (dry solids basis), preferably 0.1 to 90 wt %, more preferably 1 to 90%, and most preferably 5 to 80 wt % of the soluble α-glucan fiber composition described above.

The term “food” as used herein is intended to encompass food for human consumption as well as for animal consumption. By “functional food” it is meant any fresh or processed food claimed to have a health-promoting and/or disease-preventing and/or disease-(risk)-reducing property beyond the basic nutritional function of supplying nutrients. Functional food may include, for example, processed food or foods fortified with health-promoting additives. Examples of functional food are foods fortified with vitamins, or fermented foods with live cultures.

A carbohydrate composition comprising the soluble α-glucan fiber composition may contain other materials known in the art for inclusion in nutritional compositions, such as water or other aqueous solutions, fats, sugars, starch, binders, thickeners, colorants, flavorants, odorants, acidulants (such as lactic acid or malic acid, among others), stabilizers, or high intensity sweeteners, or minerals, among others.

Examples of suitable food products include bread, breakfast cereals, biscuits, cakes, cookies, crackers, yogurt, kefir, miso, natto, tempeh, kimchee, sauerkraut, water, milk, fruit juice, vegetable juice, carbonated soft drinks, non-carbonated soft drinks, coffee, tea, beer, wine, liquor, alcoholic drink, snacks, soups, frozen desserts, fried foods, pizza, pasta products, potato products, rice products, corn products, wheat products, dairy products, hard candies, nutritional bars, cereals, dough, processed meats and cheeses, yoghurts, ice cream confections, milk-based drinks, salad dressings, sauces, toppings, desserts, confectionery products, cereal-based snack bars, prepared dishes, and the like. The carbohydrate composition comprising the present α-glucan fiber may be in the form of a liquid, powder, tablet, cube, granule, gel, or syrup.

In certain embodiments, the carbohydrate composition according to the invention comprises at least two fiber sources (i.e., at least one additional fiber source beyond the soluble α-glucan fiber composition). In certain embodiments, one fiber source is the soluble α-glucan fiber and the second fiber source is an oligo- or polysaccharide, selected from the group consisting of resistant/branched maltodextrins/fiber dextrins (such as NUTRIOSE® from Roquette Freres, Lestrem, France; FIBERSOL-2® from ADM-Matsutani LLC, Decatur, Ill.), polydextrose (LITESSE® from Danisco—DuPont Nutrition & Health, Wilmington, Del.), soluble corn fiber (for example, PROMITOR® from Tate & Lyle, London, UK), isomaltooligosaccharides (IMOs), alternan and/or maltoalternan oligosaccharides (MAOs) (for example, FIBERMALT™ from Aevotis GmbH, Potsdam, Germany; SUCROMALT™ (from Cargill Inc., Minneapolis, Minn.), pullulan, resistant starch, inulin, fructooligosaccharides (FOS), galactooligosaccharides (GOS), xylooligosaccharides, arabinoxylooligosaccharides, nigerooligosaccharides, gentiooligosaccharides, hem icellulose and fructose oligomer syrup.

The soluble α-glucan fiber can be added to foods as a replacement or supplement for conventional carbohydrates. As such, in certain embodiments, the invention is a food product comprising the soluble α-glucan fiber. In certain embodiments, the soluble α-glucan fiber composition in the food product is produced by a process disclosed herein.

The soluble α-glucan fiber composition may be used in a carbohydrate composition and/or food product comprising one or more high intensity artificial sweeteners including, but not limited to stevia, aspartame, sucralose, neotame, acesulfame potassium, saccharin, and combinations thereof. The soluble α-glucan fiber may be blended with sugar substitutes such as brazzein, curculin, erythritol, glycerol, glycyrrhizin, hydrogenated starch hydrolysates, inulin, isomalt, lactitol, mabinlin, maltitol, maltooligosaccharide, maltoalternan oligosaccharides (such as XTEND® SUCROMALT™, available from Cargill Inc., Minneapolis, Minn.), mannitol, miraculin, a mogroside mix, monatin, monellin, osladin, pentadin, sorbitol, stevia, tagatose, thaumatin, xylitol, and any combination thereof.

In certain embodiments, a food product containing the soluble α-glucan fiber composition will have a lower glycemic response, lower glycemic index, and lower glycemic load than a similar food product in which a conventional carbohydrate is used. Further, because the soluble α-glucan fiber is characterized by very low to no digestibility in the human stomach or small intestine, in certain embodiments, the caloric content of the food product is reduced. The present soluble α-glucan fiber may be used in the form of a powder, blended into a dry powder with other suitable food ingredients or may be blended or used in the form of a liquid syrup comprising the present dietary fiber (also referred to herein as an “soluble fiber syrup”, “fiber syrup” or simply the “syrup”). The “syrup” can be added to food products as a source of soluble fiber. It can increase the fiber content of food products without having a negative impact on flavor, mouth feel, or texture.

The fiber syrup can be used in food products alone or in combination with bulking agents, such as sugar alcohols or maltodextrins, to reduce caloric content and/or to enhance nutritional profile of the product. The fiber syrup can also be used as a partial replacement for fat in food products.

The fiber syrup can be used in food products as a tenderizer or texturizer, to increase crispness or snap, to improve eye appeal, and/or to improve the rheology of dough, batter, or other food compositions. The fiber syrup can also be used in food products as a humectant, to increase product shelf life, and/or to produce a softer, moister texture. It can also be used in food products to reduce water activity or to immobilize and manage water. Additional uses of the fiber syrup may include: replacement of an egg wash and/or to enhance the surface sheen of a food product, to alter flour starch gelatinization temperature, to modify the texture of the product, and to enhance browning of the product.

The fiber syrup can be used in a variety of types of food products. One type of food product in which the present syrup can be very useful is bakery products (i.e., baked foods), such as cakes, brownies, cookies, cookie crisps, muffins, breads, and sweet doughs. Conventional bakery products can be relatively high in sugar and high in total carbohydrates. The use of the present syrup as an ingredient in bakery products can help lower the sugar and carbohydrate levels, as well as reduce the total calories, while increasing the fiber content of the bakery product.

There are two main categories of bakery products: yeast-raised and chemically-leavened. In yeast-raised products, like donuts, sweet doughs, and breads, the present fiber-containing syrup can be used to replace sugars, but a small amount of sugar may still be desired due to the need for a fermentation substrate for the yeast or for crust browning. The fiber syrup can be added with other liquids as a direct replacement for non-fiber containing syrups or liquid sweeteners. The dough would then be processed under conditions commonly used in the baking industry including being mixed, fermented, divided, formed or extruded into loaves or shapes, proofed, and baked or fried. The product can be baked or fried using conditions similar to traditional products. Breads are commonly baked at temperatures ranging from 420° F. to 520° F. (216-271° C.)°. for 20 to 23 minutes and doughnuts can be fried at temperatures ranging from 400415° F. (204-213° C.), although other temperatures and times could also be used.

Chemically leavened products typically have more sugar and may contain have a higher level of the carbohydrate compositions and/or edible syrups comprising the present soluble α-glucan fiber. A finished cookie can contain 30% sugar, which could be replaced, entirely or partially, with carbohydrate compositions and/or syrups comprising the present glucan fiber composition. These products could have a pH of 4-9.5, for example. The moisture content can be between 2-40%, for example.

The present carbohydrate compositions and/or fiber-containing syrups are readily incorporated and may be added to the fat at the beginning of mixing during a creaming step or in any method similar to the syrup or dry sweetener that it is being used to replace. The product would be mixed and then formed, for example by being sheeted, rotary cut, wire cut, or through another forming process. The products would then be baked under typical baking conditions, for example at 200-450° F. (93-232° C.).

Another type of food product in which the carbohydrate compositions and/or fiber-containing syrups can be used is breakfast cereal. For example, fiber-containing syrups could be used to replace all or part of the sugar in extruded cereal pieces and/or in the coating on the outside of those pieces. The coating is typically 30-60% of the total weight of the finished cereal piece. The syrup can be applied in a spray or drizzled on, for example.

Another type of food product in which the present α-glucan fiber composition (optionally used in the form of a carbohydrate composition and/or fiber-containing syrup) can be used is dairy products. Examples of dairy products in which it can be used include yogurt, yogurt drinks, milk drinks, flavored milks, smoothies, ice cream, shakes, cottage cheese, cottage cheese dressing, and dairy desserts, such as quarg and the whipped mousse-type products. This would include dairy products that are intended to be consumed directly (such as packaged smoothies) as well as those that are intended to be blended with other ingredients (such as blended smoothies). It can be used in pasteurized dairy products, such as ones that are pasteurized at a temperature from 160° F. to 285° F. (71-141° C.).

Another type of food product in which the composition comprising the α-glucan fiber composition can be used is confections. Examples of confections in which it can be used include hard candies, fondants, nougats and marshmallows, gelatin jelly candies or gummies, jellies, chocolate, licorice, chewing gum, caramels and toffees, chews, mints, tableted confections, and fruit snacks. In fruit snacks, a composition comprising the present α-glucan fiber could be used in combination with fruit juice. The fruit juice would provide the majority of the sweetness, and the composition comprising the glucan fiber would reduce the total sugar content and add fiber. The present compositions comprising the glucan fiber can be added to the initial candy slurry and heated to the finished solids content. The slurry could be heated from 200-305° F. (93-152° C.). to achieve the finished solids content. Acid could be added before or after heating to give a finished pH of 2-7. The composition comprising the glucan fiber could be used as a replacement for 0-100% of the sugar and 1-100% of the corn syrup or other sweeteners present.

Another type of food product in which a composition comprising the α-glucan fiber composition can be used is jams and jellies. Jams and jellies are made from fruit. A jam contains fruit pieces, while jelly is made from fruit juice. The composition comprising the present fiber can be used in place of sugar or other sweeteners as follows: weigh fruit and juice into a tank; premix sugar, the fiber-containing composition and pectin; add the dry composition to the liquid and cook to a temperature of 214-220° F. (101-104° C.); hot fill into jars and retort for 5-30 minutes.

Another type of food product in which a composition comprising the present α-glucan fiber composition (such as a fiber-containing syrup) can be used is beverages. Examples of beverages in which it can be used include carbonated beverages, fruit juices, concentrated juice mixes (e.g., margarita mix), clear waters, and beverage dry mixes. The use of the present α-glucan fiber may overcome the clarity problems that result when other types of fiber are added to beverages. A complete replacement of sugars may be possible (which could be, for example, being up to 12% or more of the total formula).

Another type of food product is high solids fillings. Examples of high solids fillings include fillings in snack bars, toaster pastries, donuts, and cookies. The high solids filling could be an acid/fruit filling or a savory filling, for example. The fiber composition could be added to products that would be consumed as is, or products that would undergo further processing, by a food processor (additional baking) or by a consumer (bake stable filling). In certain embodiments, the high solids fillings would have a solids concentration between 67-90%. The solids could be entirely replaced with a composition comprising the present α-glucan fiber or it could be used for a partial replacement of the other sweetener solids present (e.g., replacement of current solids from 5-100%). Typically fruit fillings would have a pH of 2-6, while savory fillings would be between 4-8 pH. Fillings could be prepared cold or heated at up to 250° F. (121° C.) to evaporate to the desired finished solids content.

Another type of food product in which the α-glucan fiber composition or a carbohydrate composition (comprising the α-glucan fiber composition) can be used is extruded and sheeted snacks. Examples of extruded and sheeted can be used include puffed snacks, crackers, tortilla chips, and corn chips. In preparing an extruded piece, a composition comprising the present glucan fiber would be added directly with the dry products. A small amount of water would be added in the extruder, and then it would pass through various zones ranging from 100° F. to 300° F. (38-149° C.). The dried product could be added at levels from 0-50% of the dry products mixture. A syrup comprising the present glucan fiber could also be added at one of the liquid ports along the extruder. The product would come out at either a low moisture content (5%) and then baked to remove the excess moisture, or at a slightly higher moisture content (10%) and then fried to remove moisture and cook out the product. Baking could be at temperatures up to 500° F. (260° C.). for 20 minutes. Baking would more typically be at 350° F. (177° C.) for 10 minutes. Frying would typically be at 350° F. (177° C.) for 2-5 minutes. In a sheeted snack, the composition comprising the present glucan fiber could be used as a partial replacement of the other dry ingredients (for example, flour). It could be from 0-50% of the dry weight. The product would be dry mixed, and then water added to form cohesive dough. The product mix could have a pH from 5 to 8. The dough would then be sheeted and cut and then baked or fried. Baking could be at temperatures up to 500° F. (260° C.) for 20 minutes. Frying would typically be at 350° F. (177° C.) for 2-5 minutes. Another potential benefit from the use of a composition comprising the present glucan fiber is a reduction of the fat content of fried snacks by as much as 15% when it is added as an internal ingredient or as a coating on the outside of a fried food.

Another type of food product in which a fiber-containing syrup can be used is gelatin desserts. The ingredients for gelatin desserts are often sold as a dry mix with gelatin as a gelling agent. The sugar solids could be replaced partially or entirely with a composition comprising the present glucan fiber in the dry mix. The dry mix can then be mixed with water and heated to 212° F. (100° C.). to dissolve the gelatin and then more water and/or fruit can be added to complete the gelatin dessert. The gelatin is then allowed to cool and set. Gelatin can also be sold in shelf stable packs. In that case the stabilizer is usually carrageenan-based. As stated above, a composition comprising the present glucan fiber could be used to replace up to 100% of the other sweetener solids. The dry ingredients are mixed into the liquids and then pasteurized and put into cups and allowed to cool and set.

Another type of food product in which a composition comprising the present glucan fiber can be used is snack bars. Examples of snack bars in which it can be used include breakfast and meal replacement bars, nutrition bars, granola bars, protein bars, and cereal bars. It could be used in any part of the snack bars, such as in the high solids filling, the binding syrup or the particulate portion. A complete or partial replacement of sugar in the binding syrup may be possible. The binding syrup is typically from 50-90% solids and applied at a ratio ranging from 10% binding syrup to 90% particulates, to 70% binding syrup to 30% particulates. The binding syrup is made by heating a solution of sweeteners, bulking agents and other binders (like starch) to 160-230° F. (71-110° C.) (depending on the finished solids needed in the syrup). The syrup is then mixed with the particulates to coat the particulates, providing a coating throughout the matrix. A composition comprising the present glucan fiber could also be used in the particulates themselves. This could be an extruded piece, directly expanded or gun puffed. It could be used in combination with another grain ingredient, corn meal, rice flour or other similar ingredient.

Another type of food product in which the composition comprising the present glucan fiber syrup can be used is cheese, cheese sauces, and other cheese products. Examples of cheese, cheese sauces, and other cheese products in which it can be used include lower milk solids cheese, lower fat cheese, and calorie reduced cheese. In block cheese, it can help to improve the melting characteristics, or to decrease the effect of the melt limitation added by other ingredients such as starch. It could also be used in cheese sauces, for example as a bulking agent, to replace fat, milk solids, or other typical bulking agents.

Another type of food product in which a composition comprising the present glucan fiber can be used is films that are edible and/or water soluble. Examples of films in which it can be used include films that are used to enclose dry mixes for a variety of foods and beverages that are intended to be dissolved in water, or films that are used to deliver color or flavors such as a spice film that is added to a food after cooking while still hot. Other film applications include, but are not limited to, fruit and vegetable leathers, and other flexible films.

In another embodiment, compositions comprising the present glucan fiber can be used is soups, syrups, sauces, and dressings. A typical dressing could be from 0-50% oil, with a pH range of 2-7. It could be cold processed or heat processed. It would be mixed, and then stabilizer would be added. The composition comprising the present glucan fiber could easily be added in liquid or dry form with the other ingredients as needed. The dressing composition may need to be heated to activate the stabilizer. Typical heating conditions would be from 170-200° F. (77-93° C.) for 1-30 minutes. After cooling, the oil is added to make a pre-emulsion. The product is then emulsified using a homogenizer, colloid mill, or other high shear process.

Sauces can have from 0-10% oil and from 10-50% total solids, and can have a pH from 2-8. Sauces can be cold processed or heat processed. The ingredients are mixed and then heat processed. The composition comprising the present glucan fiber could easily be added in liquid or dry form with the other ingredients as needed. Typical heating would be from 170-200° F. (77-93° C.) for 1-30 minutes.

Soups are more typically 20-50% solids and in a more neutral pH range (4-8). They can be a dry mix, to which a dry composition comprising the present glucan fiber could be added, or a liquid soup which is canned and then retorted. In soups, resistant corn syrup could be used up to 50% solids, though a more typical usage would be to deliver 5 g of fiber/serving.

Another type of food product in which a composition comprising the present α-glucan fiber composition can be used is coffee creamers. Examples of coffee creamers in which it can be used include both liquid and dry creamers. A dry blended coffee creamer can be blended with commercial creamer powders of the following fat types: soybean, coconut, palm, sunflower, or canola oil, or butterfat. These fats can be non-hydrogenated or hydrogenated. The composition comprising the present α-glucan fiber composition can be added as a fiber source, optionally together with fructo-oligosaccharides, polydextrose, inulin, maltodextrin, resistant starch, sucrose, and/or conventional corn syrup solids. The composition can also contain high intensity sweeteners, such as sucralose, acesulfame potassium, aspartame, or combinations thereof. These ingredients can be dry blended to produce the desired composition.

A spray dried creamer powder is a combination of fat, protein and carbohydrates, emulsifiers, emulsifying salts, sweeteners, and anti-caking agents. The fat source can be one or more of soybean, coconut, palm, sunflower, or canola oil, or butterfat. The protein can be sodium or calcium caseinates, milk proteins, whey proteins, wheat proteins, or soy proteins. The carbohydrate could be a composition comprising the present α-glucan fiber composition alone or in combination with fructooligosaccharides, polydextrose, inulin, resistant starch, maltodextrin, sucrose, corn syrup or any combination thereof. The emulsifiers can be mono- and diglycerides, acetylated mono- and diglycerides, or propylene glycol monoesters. The salts can be trisodium citrate, monosodium phosphate, disodium phosphate, trisodium phosphate, tetrasodium pyrophosphate, monopotassium phosphate, and/or dipotassium phosphate. The composition can also contain high intensity sweeteners, such as those describe above. Suitable anti-caking agents include sodium silicoaluminates or silica dioxides. The products are combined in slurry, optionally homogenized, and spray dried in either a granular or agglomerated form.

Liquid coffee creamers are simply a homogenized and pasteurized emulsion of fat (either dairy fat or hydrogenated vegetable oil), some milk solids or caseinates, corn syrup, and vanilla or other flavors, as well as a stabilizing blend. The product is usually pasteurized via HTST (high temperature short time) at 185° F. (85° C.) for 30 seconds, or UHT (ultra-high temperature), at 285° F. (141° C.) for 4 seconds, and homogenized in a two stage homogenizer at 500-3000 psi (3.45-20.7 MPa) first stage, and 200-1000 psi (1.38-6.89 MPa) second stage. The coffee creamer is usually stabilized so that it does not break down when added to the coffee.

Another type of food product in which a composition comprising the present α-glucan fiber composition (such as a fiber-containing syrup) can be used is food coatings such as icings, frostings, and glazes. In icings and frostings, the fiber-containing syrup can be used as a sweetener replacement (complete or partial) to lower caloric content and increase fiber content. Glazes are typically about 70-90% sugar, with most of the rest being water, and the fiber-containing syrup can be used to entirely or partially replace the sugar. Frosting typically contains about 2-40% of a liquid/solid fat combination, about 20-75% sweetener solids, color, flavor, and water. The fiber-containing syrup can be used to replace all or part of the sweetener solids, or as a bulking agent in lower fat systems.

Another type of food product in which the fiber-containing syrup can be used is pet food, such as dry or moist dog food. Pet foods are made in a variety of ways, such as extrusion, forming, and formulating as gravies. The fiber-containing syrup could be used at levels of 0-50% in each of these types.

Another type of food product in which a composition comprising the present α-glucan fiber composition, such as a syrup, can be used is fish and meat. Conventional corn syrup is already used in some meats, so a fiber-containing syrup can be used as a partial or complete substitute. For example, the syrup could be added to brine before it is vacuum tumbled or injected into the meat. It could be added with salt and phosphates, and optionally with water binding ingredients such as starch, carrageenan, or soy proteins. This would be used to add fiber, a typical level would be 5 g/serving which would allow a claim of excellent source of fiber.

Personal Care and/or Pharmaceutical Compositions Comprising the Present Soluble Fiber

The present glucan fiber and/or compositions comprising the present glucan fiber may be used in personal care products. For example, one may be able to use such materials as a humectants, hydrocolloids or possibly thickening agents. The present fibers and/or compositions comprising the present fibers may be used in conjunction with one or more other types of thickening agents if desired, such as those disclosed in U.S. Pat. No. 8,541,041, the disclosure of which is incorporated herein by reference in its entirety.

Personal care products herein include, but are not limited to, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient. An active ingredient is generally recognized as an ingredient that produces an intended pharmacological or cosmetic effect.

In certain embodiments, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil.

A personal care product, as used herein, can also be in the form of makeup or other product including, but not limited to, a lipstick, mascara, rouge, foundation, blush, eyeliner, lip liner, lip gloss, other cosmetics, sunscreen, sun block, nail polish, mousse, hair spray, styling gel, nail conditioner, bath gel, shower gel, body wash, face wash, shampoo, hair conditioner (leave-in or rinse-out), cream rinse, hair dye, hair coloring product, hair shine product, hair serum, hair anti-frizz product, hair split-end repair product, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, toothpaste, or mouthwash, for example.

A pharmaceutical product, as used herein, can be in the form of an emulsion, liquid, elixir, gel, suspension, solution, cream, capsule, tablet, sachet or ointment, for example. Also, a pharmaceutical product herein can be in the form of any of the personal care products disclosed herein. A pharmaceutical product can further comprise one or more pharmaceutically acceptable carriers, diluents, and/or pharmaceutically acceptable salts. The present fibers and/or compositions comprising the present fibers can also be used in capsules, encapsulants, tablet coatings, and as an excipients for medicaments and drugs.

### Enzymatic Synthesis of the Soluble α-Glucan Fiber Composition

Methods are provided to enzymatically produce a soluble α-glucan fiber composition. Two different methods are described herein. In an embodiment, the “single enzyme” method comprises the use of at least one glucosyltransferase (in the absence of an α-glucanohydrolase) belonging to the glucoside hydrolase type 70 family (E.C. 2.4.1.-) and which is capable of catalyzing the synthesis of a digestion resistant soluble α-glucan fiber composition using sucrose as a substrate. In another embodiment, a “two enzyme” method comprises a combination of at least one glucosyltransferase (GH70) in combination with at least one α-glucanohydrolase (such as an endomutanase).

Glycoside hydrolase family 70 enzymes are transglucosidases produced by lactic acid bacteria such as Streptococcus, Leuconostoc, Weise/la or Lactobacillus genera (see Carbohydrate Active Enzymes database; “CAZy”; Cantarel et al., (2009) Nucleic Acids Res 37:D233-238). The recombinantly expressed glucosyltransferases preferably have an amino acid sequence identical to that found in nature (i.e., the same as the full length sequence as found in the source organism or a catalytically active truncation thereof).

GTF enzymes are able to polymerize the D-glucosyl units of sucrose to form homooligosaccharides or homopolysaccharides. Depending upon the specificity of the GTF enzyme, linear and/or branched glucans comprising various glycosidic linkages are formed such as α-(1,2), α-(1,3), α-(1,4) and α-(1,6). Glucosyltransferases may also transfer the D-glucosyl units onto hydroxyl acceptor groups. A non-limiting list of acceptors include carbohydrates, alcohols, polyols or flavonoids. The structure of the resultant glucosylated product is dependent upon the enzyme specificity.

In the present invention the D-glucopyranosyl donor is sucrose. As such the reaction is:

Sucrose+GTF [symbol] α-D-(Glucose)ₙ+D-Fructose+GTF

The type of glycosidic linkage predominantly formed is used to name/classify the glucosyltransferase enzyme. Examples include dextransucrases (α-(1,6) linkages; EC 2.4.1.5), mutansucrases (α-(1,3) linkages; EC 2.4.1.-), alternansucrases (alternating α(1,3)-α(1,6) backbone; EC 2.4.1.140), and reuteransucrases (mix of α-(1,4) and α-(1,6) linkages; EC 2.4.1.-).

In one aspect, the glucosyltransferase (GTF) is capable of forming glucans having α-(1,3) glycosidic linkages with the proviso that the glucan product is not an alternan (i.e., the enzyme is not an alternansucrase).

In one aspect, the glucosyltransferase comprises an amino acid sequence having at least 90% identity, preferably at least 91, 92, 93, 94, 95, 96, 97, 98, 99 or 100% identity to SEQ ID NO: 1, 3, 13, 16, 17, 19, 28, 30, 32, 34, 36, 38, 40, 42, 44, 46, 48, 50, 52, 54, 56, 58, 60, or 62. In a preferred aspect, the glucosyltransferase comprises an amino acid sequence selected from the group consisting of SEQ ID NOs: 1, 3, 13, 16, 17, 19, 28, 30, 32, 34, 36, 38, 40, 42, 44, 46, 48, 50, 52, 54, 56, 58, 60, and 62. However, it should be noted that some wild type sequences may be found in nature in a truncated form. As such, and in a further embodiment, the glucosyltransferase suitable for use may be a truncated form of the wild type sequence. In a further embodiment, the truncated glucosyltransferase comprises a sequence derived from the full length wild type amino acid sequence selected from the group consisting of SEQ ID NOs: 1, 13, 17, 28, 30, 32, 34, 36, 38, 40, 42, 44, and 46. In another embodiment, the glucosyltransferase may be truncated and will have an amino acid sequence selected from the group consisting of SEQ ID NOs: 3, 16, 19, 48, 50, 52, 54, 56, 58, 60, and 62.

The concentration of the catalyst in the aqueous reaction formulation depends on the specific catalytic activity of the catalyst, and is chosen to obtain the desired rate of reaction. The weight of each catalyst (either a single glucosyltransferase or individually a glucosyltransferase and α-glucanohydrolase) reactions typically ranges from 0.0001 mg to 20 mg per mL of total reaction volume, preferably from 0.001 mg to 10 mg per mL. The catalyst may also be immobilized on a soluble or insoluble support using methods well-known to those skilled in the art; see for example, Immobilization of Enzymes and Cells; Gordon F. Bickerstaff, Editor; Humana Press, Totowa, N.J., USA; 1997. The use of immobilized catalysts permits the recovery and reuse of the catalyst in subsequent reactions. The enzyme catalyst may be in the form of whole microbial cells, permeabilized microbial cells, microbial cell extracts, partially-purified or purified enzymes, and mixtures thereof.

The pH of the final reaction formulation is from about 3 to about 8, preferably from about 4 to about 8, more preferably from about 5 to about 8, even more preferably about 5.5 to about 7.5, and yet even more preferably about 5.5 to about 6.5. The pH of the reaction may optionally be controlled by the addition of a suitable buffer including, but not limited to, phosphate, pyrophosphate, bicarbonate, acetate, or citrate. The concentration of buffer, when employed, is typically from 0.1 mM to 1.0 M, preferably from 1 mM to 300 mM, most preferably from 10 mM to 100 mM.

The sucrose concentration initially present when the reaction components are combined is at least 50 g/L, preferably 50 g/L to 600 g/L, more preferably 100 g/L to 500 g/L, more preferably 150 g/L to 450 g/L, and most preferably 250 g/L to 450 g/L. The substrate for the α-glucanohydrolase (when present) will be the members of the glucose oligomer population formed by the glucosyltransferase. As the glucose oligomers present in the reaction system may act as acceptors, the exact concentration of each species present in the reaction system will vary. Additionally, other acceptors may be added (i.e., external acceptors) to the initial reaction mixture such as maltose, isomaltose, isomaltotriose, and methyl-α-D-glucan, to name a few.

The length of the reaction may vary and may often be determined by the amount of time it takes to use all of the available sucrose substrate. In one embodiment, the reaction is conducted until at least 90%, preferably at least 95% and most preferably at least 99% of the sucrose initially present in the reaction mixture is consumed. In another embodiment, the reaction time is 1 hour to 168 hours, preferably 1 hour to 72 hours, and most preferably 1 hour to 24 hours.

### Single Enzyme Method (Glucosyltransferase)

Two glucosyltransferases/glucansucrases have been identified capable of producing the present α-glucan fiber composition in the absence of an α-glucanohydrolase. Specifically, a glucosyltransferase from

Streptococcus mutans (GENBANK® gi: 3130088 (or a catalytically active truncation thereof suitable for expression in the recombinant microbial host cell); also referred to herein as the “0088” glucosyltransferase or “GTF0088”) can produce the present α-glucan fiber composition. In one aspect, the Streptococcus mutans GTF0088 may be produced as a catalytically active fragment of the full length sequence reported in GENBANK® gi: 3130088. In one embodiment, the present α-glucan fiber composition is produced using the Streptococcus mutans GTF0088 glucosyltransferase or a catalytically active fragment thereof.

In one embodiment, a method to produce an α-glucan fiber composition is provided comprising:

a. providing a set of reaction components comprising:
i. sucrose;
ii. at least one polypeptide having glucosyltransferase activity and comprising an amino acid sequence having at least 90% identity to a sequence selected from SEQ ID NOs: 13, 16, 17, 19, 28, 30, 32, 34, 36, 38, 40, 42, 44, 46, 48, 50, 52, 54, 56, 58, 60, and 62; and
iii. optionally one or more acceptors;

b. combining the set of reaction components under suitable aqueous reaction conditions to form a single reaction mixture, whereby a product mixture comprising glucose oligomers is formed;

c. optionally isolating the soluble α-glucan fiber composition described above from the product mixture comprising glucose oligomers; and

d. optionally concentrating the soluble α-glucan fiber composition.

In a preferred embodiment, the present α-glucan fiber composition is produced using a glucosyltransferase enzyme comprising an amino acid sequence having at least 90%, preferably 91, 92, 93, 94, 95, 96, 97, 98, 99, or 100% to SEQ ID NO: 13 (the full length form) or SEQ ID NO: 16, 48, or 56 (catalytically active truncated forms) with the understanding that such enzymes will retain a similar activity and produce a product profile consistent with the present α-glucan fiber composition.

In another embodiment, a glucosyltransferase from Streptococcus mutans 1123 GENBANK® gi:387786207 (or a catalytically active truncation thereof suitable for expression in the recombinant microbial host cell; herein also referred to as the “6207” glucosyltransferase or simply “GTF6207”) has also been identified as being capable of producing the present α-glucan fiber composition in the absence of an α-glucanohydrolase (e.g., dextranase, mutanase, etc.). In one aspect, the Streptococcus mutan GTF6207 may be produced as a catalytically active fragment of the full length sequence reported in GENBANK® gi: 387786207. In one embodiment, the present α-glucan fiber composition is produced using the Streptococcus mutans GTF6207 glucosyltransferase or a catalytically active fragment thereof. In a preferred embodiment, the present α-glucan fiber composition is produced using a glucosyltransferase enzyme having an amino acid sequence having at least 90%, preferably 91, 92, 93, 94, 95, 96, 97, 98, 99, or 100% to SEQ ID NO: 17 (the full length form) or SEQ ID NO: 19 (a catalytically active truncated form) with the understanding that such enzymes will retain a similar activity and produce a product profile consistent with the present α-glucan fiber composition.

In further embodiments, the present α-glucan fiber composition is produced using a glucosyltransferase enzyme having an amino acid sequence having at least 90%, preferably 91, 92, 93, 94, 95, 96, 97, 98, 99, or 100% to a homolog or a truncation of a homolog of SEQ ID NO: 13 with the understanding that such enzymes will retain a similar activity and produce a product profile consistent with the present α-glucan fiber composition. In certain embodiments, the homolog is selected from SEQ ID NOs: 28, 30, 32, 34, 36, 40, 42, 44, and 46. In certain embodiments, the truncation of a homolog is selected from SEQ ID NOs: 50, 52, 54, 58, 60, and 62.

### Soluble Glucan Fiber Synthesis—Reaction Systems Comprising a Glucosyltransferase (Gtf) and an α-Glucanohydrolase

A method is provided to enzymatically produce the present soluble glucan fibers using at least one α-glucanohydrolase in combination (i.e., concomitantly in the reaction mixture) with at least one of the above glucosyltransferases. The simultaneous use of the two enzymes produces a different product profile (i.e., the profile of the soluble fiber composition) when compared to a sequential application of the same enzymes (i.e., first synthesizing the glucan polymer from sucrose using a glucosyltransferase and then subsequently treating the glucan polymer with an α-glucanohydrolase). In one embodiment, a glucan fiber synthesis method based on sequential application of a glucosyltransferase with an α-glucanohydrolase is specifically excluded.

In one embodiment, a method to produce a soluble α-glucan fiber composition is provided comprising:

a. providing a set of reaction components comprising:
i. sucrose;
ii. at least one polypeptide having glucosyltransferase activity, said polypeptide comprising an amino acid sequence having at least 90% identity to a sequence selected from SEQ ID NOs: 1 and 3;
iii. at least one polypeptide having α-glucanohydrolase activity; and
iv. optionally one more acceptors;

b. combining the set of reaction componenets under suitable aqueous reaction conditions whereby a product comprising a soluble α-glucan fiber composition is produced; and

c. optionally isolating the soluble α-glucan fiber composition from the product of step (b).

A glucosyltransferase from Streptococcus mutans NN2025 (GENBANK® GI:290580544; also referred to herein as the “0544” glucosyltransferase or simply “GTF0544”) can produce the present α-glucan fiber composition when used in combination with an α-glucanohydrolase having endohydrolytic activity. In one aspect, the Streptococcus mutans GTF0544 may be produced as a catalytically active fragment of the full length sequence reported in GENBANK® gi: 290580544. In one embodiment, the present α-glucan fiber composition is produced using the Streptococcus mutans GTF0544 glucosyltransferase (or a catalytically active fragment thereof suitable for expression in the recombinant host cell) in combination with a least one α-glucanohydrolase having endohydrolytic activity. Similar to the glucosyltransferases, an α-glucanohydrolase may be defined by the endohydrolysis activity towards certain α-D-glycosidic linkages. α-glucanohydrolases useful in the methods disclosed herein can be identified by their characteristic domain structures, for example, those domain structures identified for mutanases and dextranases described above. Examples may include, but are not limited to, dextranases (capable of hydrolyzing α-(1,6)-linked glycosidic bonds; E.C. 3.2.1.11), mutanases (capable of hydrolyzing α-(1,3)-linked glycosidic bonds; E.C. 3.2.1.59), mycodextranases (capable of endohydrolysis of (1→4)-α-D-glucosidic linkages in α-D-glucans containing both (1→3)- and (1→4)-bonds; EC 3.2.1.61), glucan 1,6-α-glucosidase (EC 3.2.1.70), and alternanases (capable of endohydrolytically cleaving alternan; E.C. 3.2.1.-; see U.S. Pat. No. 5,786,196). Various factors including, but not limited to, level of branching, the type of branching, and the relative branch length within certain α-glucans may adversely impact the ability of an α-glucanohydrolase to endohydrolyze some glycosidic linkages.

In one embodiment, the α-glucanohydrolase is at least one mutanase (EC 3.1.1.59). Mutanases useful in the methods disclosed herein can be identified by their characteristic structure. See, e.g., Y. Hakamada et al. (Biochimie, (2008) 90:525-533). In an embodiment, the mutanase is one obtainable from the genera Penicillium, Paenibacillus, Hypocrea, Aspergillus, and Trichoderma. In a further embodiment, the mutanase is from Penicillium marneffei ATCC 18224 or Paenibacillus Humicus. In one embodiment, the mutanase comprises an amino acid sequence selected from SEQ ID NOs 4, 6, 9, 11, and any combination thereof. In another embodiment, the above mutanases may be a catalytically active truncation so long as the mutanase activity is retained. In a preferred embodiment, the Paenibacillus Humicus mutanase, identified in GENBANK® as gi:257153264 (also referred to herein as the “3264” mutanase or simply “MUT3264”) or a catalytically active fragment thereof may be used in combination with the GTF0544 glucosyltransferase to produce the present α-glucan fiber composition. The MUT3264 mutanase may be produced with its native signal sequence, an alternative signal sequence (such as the Bacillus subtilis AprE signal sequence; SEQ ID NO: 7), or may be produced in a mature form (for example, a truncated form lacking the signal sequence) so long as the desired mutanase activity is retained and the resulting product (when used in combination with the GTF0544 glucosyltransferase) is the present α-glucan fiber composition.

In a preferred embodiment, the present α-glucan fiber composition is produced using a glucosyltransferase enzyme having an amino acid sequence having at least 90%, preferably 91, 92, 93, 94, 95, 96, 97, 98, 99, or 100% to SEQ ID NO: 1 (the full length form) or SEQ ID NO: 3 (a catalytically active truncated form) in combination with a mutanase having at least 90%, preferably 91, 92, 93, 94, 95, 96, 97, 98, 99, or 100% to SEQ ID NO: 4 (the full length form as reported in GENBANK® gi: 257153264) or SEQ ID NO: 6 or SEQ ID NO: 9 with the understanding that the combinations of enzymes (GTF0544 and MUT3264) will retain a similar activity and produce a product profile consistent with the present α-glucan fiber composition.

The temperature of the enzymatic reaction system comprising concomitant use of at least one glucosyltransferase and at least one α-glucanohydrolase may be chosen to control both the reaction rate and the stability of the enzyme catalyst activity. The temperature of the reaction may range from just above the freezing point of the reaction formulation (approximately 0° C.) to about 60° C., with a preferred range of 5° C. to about 55° C., and a more preferred range of reaction temperature of from about 20° C. to about 47° C.

The ratio of glucosyltransferase activity to α-glucanohydrolase activity may vary depending upon the selected enzymes. In one embodiment, the ratio of glucosyltransferase to α-glucanohydrolase ranges from 1:0.01 to 0.01:1.0.

### Methods to Identify Substantially Similar Enzymes Having the Desired Activity

The skilled artisan recognizes that substantially similar enzyme sequences may also be used in the present compositions and methods so long as the desired activity is retained (i.e., glucosyltransferase activity capable of forming glucans having the desired glycosidic linkages or α-glucanohydrolases having endohydrolytic activity towards the target glycosidic linkage(s)). For example, it has been demonstrated that catalytically active truncations may be prepared and used so long as the desired activity is retained (or even improved in terms of specific activity). In one embodiment, substantially similar sequences are defined by their ability to hybridize, under highly stringent conditions with the nucleic acid molecules associated with sequences exemplified herein. In another embodiment, sequence alignment algorithms may be used to define substantially similar enzymes based on the percent identity to the DNA or amino acid sequences provided herein.

As used herein, a nucleic acid molecule is “hybridizable” to another nucleic acid molecule, such as a cDNA, genomic DNA, or RNA, when a single strand of the first molecule can anneal to the other molecule under appropriate conditions of temperature and solution ionic strength. Hybridization and washing conditions are well known and exemplified in Sambrook, J. and Russell, D., T. Molecular Cloning: A Laboratory Manual, Third Edition, Cold Spring Harbor Laboratory Press, Cold Spring Harbor (2001). The conditions of temperature and ionic strength determine the “stringency” of the hybridization. Stringency conditions can be adjusted to screen for moderately similar molecules, such as homologous sequences from distantly related organisms, to highly similar molecules, such as genes that duplicate functional enzymes from closely related organisms. Post-hybridization washes typically determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of conditions uses higher temperatures in which the washes are identical to those above except for the temperature of the final two 30 min washes in 0.2×SSC, 0.5% SDS was increased to 60° C. Another preferred set of highly stringent hybridization conditions is 0.1×SSC, 0.1% SDS, 65° C. and washed with 2×SSC, 0.1° A SDS followed by a final wash of 0.1×SSC, 0.1% SDS, 65° C.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (Sambrook, J. and Russell, D., T., supra). For hybridizations with shorter nucleic acids, i.e., oligonucleotides, the position of mismatches becomes more important, and the length of the oligonucleotide determines its specificity. In one aspect, the length for a hybridizable nucleic acid is at least about 10 nucleotides. Preferably, a minimum length for a hybridizable nucleic acid is at least about 15 nucleotides in length, more preferably at least about 20 nucleotides in length, even more preferably at least 30 nucleotides in length, even more preferably at least 300 nucleotides in length, and most preferably at least 800 nucleotides in length. Furthermore, the skilled artisan will recognize that the temperature and wash solution salt concentration may be adjusted as necessary according to factors such as length of the probe.

As used herein, the term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs. Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, Md.), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment is preferred. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5.

In one aspect, suitable isolated nucleic acid molecules encode a polypeptide having an amino acid sequence that is at least about 20%, preferably at least 30%, 40%, 50%, 60%, 70%, 80%, 85%, 90%, 91° A, 92%, 93%, 94%, 95%, 96%, 97%, 98%, or 99% identical to an amino acid sequence reported herein. In another aspect, suitable isolated nucleic acid molecules encode a polypeptide having an amino acid sequence that is at least about 20%, preferably at least 30%, 40%, 50%, 60%, 70%, 80%, 85%, 90%, 91%, 92%, 93%, 94%, 95%, 96%, 97%, 98%, or 99% identical to an amino acid sequence reported herein; with the proviso that the polypeptide retains the respective activity (i.e., glucosyltransferase or α-glucanohydrolase activity).

### Gas Production

A rapid rate of gas production in the lower gastrointestinal tract gives rise to gastrointestinal discomfort such as flatulence and bloating, whereas if gas production is gradual and low the body can more easily cope. For example, inulin gives a boost of gas production which is rapid and high when compared to the present glucan fiber composition at an equivalent dosage (grams soluble fiber), whereas the present glucan fiber composition preferably has a rate of gas release that is lower than that of inulin at an equivalent dosage.

In one embodiment, consumption of food products containing the soluble α-glucan fiber composition disclosed herein results in a rate of gas production that is well tolerated for food applications. In one embodiment, the relative rate of gas production is no more than the rate observed for inulin under similar conditions, preferably the same or less than inulin, more preferably less than inulin, and most preferably much less than inulin at an equivalent dosage. In another embodiment, the relative rate of gas formation is measured over 3 hours or 24 hours using the methods described herein. In a preferred aspect, the rate of gas formation is at least 1%, preferably 2%, 3%, 4%, 5%, 6%, 7%, 8%, 9%, 10%, 15%, 20%, 25% or at least 30% less than the rate observed for inulin under the same reaction conditions.

### Beneficial Physiological Properties

### Short Chain Fatty Acid Production

Use of the compounds according to the present invention may facilitate the production of energy yielding metabolites through colonic fermentation. Use of compounds according to the invention may facilitate the production of short chain fatty acids (SCFAs), such as propionate and/or butyrate. SCFAs are known to lower cholesterol. Consequently, the compounds of the invention may lower the risk of developing high cholesterol. The present glucan fiber composition may stimulate the production of SCFAs, especially proprionate and/or butyrate, in fermentation studies. As the production of SCFA or the increased ratio of SCFA to acetate is beneficial for the control of cholesterol levels in a mammal in need thereof, the disclosed fiber composition may be of particular interest to nutritionists and consumers for the prevention and/or treatment of cardiovascular risks. Thus, another aspect, the disclosure provides a method for improving the health of a subject comprising administering a composition comprising the present α-glucan fiber composition to a subject in an amount effective to exert a beneficial effect on the health of said subject, such as for treating cholesterol-related diseases. In addition, it is generally known that SCFAs lower the pH in the gut and this helps calcium absorption. Thus, compounds according to the present disclosure may also affect mineral absorption. This means that they may also improve bone health, or prevent or treat osteoporosis by lowering the pH due to SCFA increases in the gut. The production of SCFA may increase viscosity in small intestine which reduces the re-absorption of bile acids; increasing the synthesis of bile acids from cholesterol and reduces circulating low density lipoprotein (LDL) cholesterol.

An “effective amount” of a compound or composition as defined herein refers to an amount effective, at dosages and for periods of time necessary, to achieve a desired beneficial physiological effect, such as lowering of blood cholesterol, increasing short chain fatty acid production or preventing or treating a gastrointestinal disorder. For instance, the amount of a composition administered to a subject will vary depending upon factors such as the subject's condition, the subject's body weight, the age of the subject, and whether a composition is the sole source of nutrition. The effective amount may be readily set by a medical practitioner or dietician. In general, a sufficient amount of the composition is administered to provide the subject with up to about 50 g of dietary fiber (insoluble and soluble) per day; for example about 25 g to about 35 g of dietary fiber per day. The amount of the present soluble α-glucan fiber composition that the subject receives is preferably in the range of about 0.1 g to about 50 g per day, more preferably in the rate of 0.5 g to 20 g per day, and most preferably 1 to 10 g per day. A compound or composition as defined herein may be taken in multiple doses, for example 1 to 5 times, spread out over the day or acutely, or may be taken in a single dose. A compound or composition as defined herein may also be fed continuously over a desired period. In certain embodiments, the desired period is at least one week or at least two weeks or at least three weeks or at least one month or at least six months.

In a preferred embodiment, the present disclosure provides a method for decreasing blood triglyceride levels in a subject in need thereof by administering a compound or a composition as defined herein to a subject in need thereof. In another preferred embodiment, the disclosure provides a method for decreasing low density lipoprotein levels in a subject in need thereof by administering a compound or a composition as defined herein to a subject in need thereof. In another preferred embodiment, the disclosure provides a method for increasing high density lipoprotein levels in a subject in need thereof by administering a compound or a composition as defined herein to a subject in need thereof.

### Attenuation of Postprandial Blood Glucose Concentrations/Glycemic Response

The presence of bonds other than α-(1,4) backbone linkages in the present α-glucan fiber composition provides improved digestion resistance as enzymes of the human digestion track may have difficultly hydrolyzing such bonds and/or branched linkages. The presence of branches provides partial or complete indigestibility to glucan fibers, and therefore virtually no or a slower absorption of glucose into the body, which results in a lower glycemic response. Accordingly, the present disclosure provides an α-glucan fiber composition for the manufacture of food and drink compositions resulting in a lower glycemic response. For example, these compounds can be used to replace sugar or other rapidly digestible carbohydrates, and thereby lower the glycemic load of foods, reduce calories, and/or lower the energy density of foods. Also, the stability of the present α-glucan fiber composition possessing these types of bonds allows them to be easily passed through into the large intestine where they may serve as a substrate specific for the colonic microbial flora.

### Improvement of Gut Health

In a further embodiment, compounds as disclosed herein may be used for the treatment and/or improvement of gut health. The present α-glucan fiber composition is preferably slowly fermented in the gut by the gut microflora. Preferably, the present compounds exhibit in an in vitro gut model a tolerance no worse than inulin or other commercially available fibers such as PROMITOR® (soluble corn fiber, Tate & Lyle), NUTRIOSE® (soluble corn fiber or dextrin, Roquette), or FIBERSOL®-2 (digestion-resistant maltodextrin, Archer Daniels Midland Company & Matsutani Chemical), (i.e., similar level of gas production), preferably an improved tolerance over one or more of the commercially available fibers, i.e. the fermentation of the present glucan fiber results in less gas production than inulin in 3 hours or 24 hours, thereby lowering discomfort, such as flatulence and bloating, due to gas formation. In one aspect, the disclosure also relates to a method for moderating gas formation in the gastrointestinal tract of a subject by administering a compound or a composition as disclosed herein to a subject in need thereof, so as to decrease gut pain or gut discomfort due to flatulence and bloating. In further embodiments, compositions as disclosed herein provide subjects with improved tolerance to food fermentation, and may be combined with fibers, such as inulin or FOS, GOS, or lactulose to improve tolerance by lowering gas production.

In another embodiment, compounds as disclosed herein may be administered to improve laxation or improve regularity by increasing stool bulk.

### Prebiotics and Probiotics

The soluble α-glucan fiber composition(s) may be useful as prebiotics, or as “synbiotics” when used in combination with probiotics, as discussed below. By “prebiotic” it is meant a food ingredient that beneficially affects the subject by selectively stimulating the growth and/or activity of one or a limited number of bacteria in the gastrointestinal tract, particularly the colon, and thus improves the health of the host. Examples of prebiotics include fructooligosaccharides, inulin, polydextrose, resistant starch, soluble corn fiber, glucooligosaccharides and galactooligosaccharides, arabinoxylan-oligosaccharides, lactitol, and lactulose.

In another embodiment, compositions comprising the soluble α-glucan fiber composition further comprise at least one probiotic organism.

By “probiotic organism” it is meant living microbiological dietary supplements that provide beneficial effects to the subject through their function in the digestive tract. In order to be effective the probiotic microorganisms must be able to survive the digestive conditions, and they must be able to colonize the gastrointestinal tract at least temporarily without any harm to the subject. Only certain strains of microorganisms have these properties. Preferably, the probiotic microorganism is selected from the group comprising Lactobacillus spp., Bifidobacterium spp., Bacillus spp., Enterococcus spp., Escherichia spp., Streptococcus spp., and Saccharomyces spp. Specific microorganisms include, but are not limited to Bacillus subtilis, Bacillus cereus, Bifidobacterium bificum, Bifidobacterium breve, Bifidobacterium infantis, Bifidobacterium lactis, Bifidobacterium longum, Bifidobacterium thermophilum, Enterococcus faecium, Enterococcus faecium, Lactobacillus acidophilus, Lactobacillus bulgaricus, Lactobacillus casei, Lactobacillus lactis, Lactobacillus plantarum, Lactobacillus reuteri, Lactobacillus rhamnosus, Streptococcus faecium, Streptococcus mutans, Streptococcus thermophilus, Saccharomyces boulardii, Torulopsia, Aspergillus oryzae, and Streptomyces among others, including their vegetative spores, non-vegetative spores (Bacillus) and synthetic derivatives. More preferred probiotic microorganisms include, but are not limited to members of three bacterial genera: Lactobacillus, Bifidobacterium and Saccharomyces. In a preferred embodiment, the probiotic microorganism is Lactobacillus, Bifidobacterium, and a combination thereof

The probiotic organism can be incorporated into the composition as a culture in water or another liquid or semisolid medium in which the probiotic remains viable. In another technique, a freeze-dried powder containing the probiotic organism may be incorporated into a particulate material or liquid or semi-solid material by mixing or blending.

In a preferred embodiment, the composition comprises a probiotic organism in an amount sufficient to delivery at least 1 to 200 billion viable probiotic organisms, preferably 1 to 100 billion, and most preferably 1 to 50 billion viable probiotic organisms. The amount of probiotic organisms delivery as describe above is may be per dosage and/or per day, where multiple dosages per day may be suitable for some applications. Two or more probiotic organisms may be used in a composition.

### Methods to Obtain the Enzymatically-Produced Soluble α-Glucan Fiber Composition

Any number of common purification techniques may be used to obtain the present soluble α-glucan fiber composition from the reaction system including, but not limited to centrifugation, filtration, fractionation, chromatographic separation, dialysis, evaporation, precipitation, dilution or any combination thereof, preferably by dialysis or chromatographic separation, most preferably by dialysis (ultrafiltration).

### Recombinant Microbial Expression

The genes and gene products of the instant sequences may be produced in heterologous host cells, particularly in the cells of microbial hosts. Preferred heterologous host cells for expression of the instant genes and nucleic acid molecules are microbial hosts that can be found within the fungal or bacterial families and which grow over a wide range of temperature, pH values, and solvent tolerances. For example, it is contemplated that any of bacteria, yeast, and filamentous fungi may suitably host the expression of the present nucleic acid molecules. The enzyme(s) may be expressed intracellularly, extracellularly, or a combination of both intracellularly and extracellularly, where extracellular expression renders recovery of the desired protein from a fermentation product more facile than methods for recovery of protein produced by intracellular expression. Transcription, translation and the protein biosynthetic apparatus remain invariant relative to the cellular feedstock used to generate cellular biomass; functional genes will be expressed regardless. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, the fungal host cell is Trichoderma, preferably a strain of Trichoderma reesei. In one embodiment, bacterial host strains include Escherichia, Bacillus, Kluyveromyces, and Pseudomonas. In a preferred embodiment, the bacterial host cell is Bacillus subtilis or Escherichia coli.

Large-scale microbial growth and functional gene expression may use a wide range of simple or complex carbohydrates, organic acids and alcohols or saturated hydrocarbons, such as methane or carbon dioxide in the case of photosynthetic or chemoautotrophic hosts, the form and amount of nitrogen, phosphorous, sulfur, oxygen, carbon or any trace micronutrient including small inorganic ions. The regulation of growth rate may be affected by the addition, or not, of specific regulatory molecules to the culture and which are not typically considered nutrient or energy sources.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell and/or native to the production host, although such control regions need not be so derived.

Initiation control regions or promoters which are useful to drive expression of the present cephalosporin C deacetylase coding region in the desired host cell are numerous and familiar to those skilled in the art. Virtually any promoter capable of driving these genes is suitable for the present invention including but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred host cell. In one embodiment, the inclusion of a termination control region is optional. In another embodiment, the chimeric gene includes a termination control region derived from the preferred host cell.

### Industrial Production

A variety of culture methodologies may be applied to produce the enzyme(s). For example, large-scale production of a specific gene product over-expressed from a recombinant microbial host may be produced by batch, fed-batch, and continuous culture methodologies. Batch and fed-batch culturing methods are common and well known in the art and examples may be found in Biotechnology: A Textbook of Industrial Microbiology by Wulf Crueger and Anneliese Crueger (authors), Second Edition, (Sinauer Associates, Inc., Sunderland, Mass. (1990) and Manual of Industrial Microbiology and Biotechnology, Third Edition, Richard H. Baltz, Arnold L. Demain, and Julian E. Davis (Editors), (ASM Press, Washington, D.C. (2010).

Commercial production of the desired enzyme(s) may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added and valuable products, by-products or waste products are continuously removed from the cell mass. Cell immobilization may be performed using a wide range of solid supports composed of natural and/or synthetic materials.

Recovery of the desired enzyme(s) from a batch fermentation, fed-batch fermentation, or continuous culture, may be accomplished by any of the methods that are known to those skilled in the art. For example, when the enzyme catalyst is produced intracellularly, the cell paste is separated from the culture medium by centrifugation or membrane filtration, optionally washed with water or an aqueous buffer at a desired pH, then a suspension of the cell paste in an aqueous buffer at a desired pH is homogenized to produce a cell extract containing the desired enzyme catalyst. The cell extract may optionally be filtered through an appropriate filter aid such as celite or silica to remove cell debris prior to a heat-treatment step to precipitate undesired protein from the enzyme catalyst solution. The solution containing the desired enzyme catalyst may then be separated from the precipitated cell debris and protein by membrane filtration or centrifugation, and the resulting partially-purified enzyme catalyst solution concentrated by additional membrane filtration, then optionally mixed with an appropriate carrier (for example, maltodextrin, phosphate buffer, citrate buffer, or mixtures thereof) and spray-dried to produce a solid powder comprising the desired enzyme catalyst. Alternatively, the resulting partially-purified enzyme catalyst solution can be stabilized as a liquid formulation by the addition of polyols such as maltodextrin, sorbitol, or propylene glycol, to which is optionally added a preservative such as sorbic acid, sodium sorbate or sodium benzoate.

The production of the soluble α-glucan fiber can be carried out by combining the obtained enzyme(s) under any suitable aqueos reaction conditions which result in the production of the soluble α-glucan fiber such as the conditions disclosed herein. The reaction may be carried out in water solution, or, in certain embodiments, the reaction can be carried out in situ within a food product. Methods for producing a fiber using an enzyme catalyst in situ in a food product are known in the art. In certain embodiments, the enzyme catalyst is added to a sucrose-containing liquid food product. The enzyme catalyst can reduce the amount of sucrose in the liquid food product while increasing the amount of soluble α-glucan fiber and fructose. A suitable method for in situ production of fiber using a polypeptide material (i.e., an enzyme catalyst) within a food product can be found in WO2013/182686, the contents of which are herein incorporated by reference for the disclosure of a method for in situ production of fiber in a food product using an enzyme catalyst.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

### DESCRIPTION OF CERTAIN EMBODIMENTS

In a first embodiment, a soluble α-glucan fiber composition is provided, said soluble α-glucan fiber composition comprising:

a. 10-30% α-(1,3) glycosidic linkages;

b. 65-87% α-(1,6) glycosidic linkages;

c. less than 5% α-(1,3,6) glycosidic linkages;

d. a weight average molecular weight of less than 5000 Daltons;

e. a viscosity of less than 0.25 Pascal second (Pa·s) at 12 wt % in water at 20° C.;

f. a dextrose equivalence (DE) in the range of 4 to 40; and

g. a digestibility of less than 12% as measured by the Association of Analytical Communities (AOAC) method 2009.01;

h. a solubility of at least 20% (w/w) in pH 7 water at 25° C.; and

i. a polydispersity index of less than 5.

In another embodiment to any of the above embodiments, the present soluble α-glucan fiber composition comprises less than 10% reducing sugars.

In another embodiment to any of the above embodiments, the soluble α-glucan fiber composition comprises less than 1% α-(1,4) glycosidic linkages.

In another embodiment to any of the above embodiments, the soluble α-glucan fiber composition is characterized by a number average molecular weight (Mn) between 400 and 2000 g/mole.

In one embodiment, a carbohydrate composition is provided comprising: 0.01 to 99 wt %, preferably 10 to 90 wt %, (dry solids basis) of the soluble α-glucan fiber composition of the first embodiment.

In another embodiment to any of the above embodiments, the carbohydrate composition comprises: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

In another embodiment to any of the above embodiments, the carbohydrate composition is in the form of a liquid, a syrup, a powder, granules, shaped spheres, shaped sticks, shaped plates, shaped cubes, tablets, capsules, sachets, or any combination thereof.

In another embodiment, a food product, a personal care product, or pharmaceutical product is provided which comprises the soluble α-glucan fiber composition of the first embodiment or a carbohydrate composition comprising the soluble α-glucan fiber composition of the first embodiment.

In another embodiment, a method to produce a soluble α-glucan fiber composition is provided comprising:

a. providing a set of reaction components comprising:
i. sucrose; preferably at a concentration of at least 50 g/L, preferably at least 200 g/L;
ii. at least one polypeptide having glucosyltransferase activity, said polypeptide comprising an amino acid sequence having at least 90% identity, preferably at leat 91, 92, 93, 94, 95, 96, 97, 98, 99 or 100% identity to SEQ ID NO: 1 or 3;
iii. at least one polypeptide having α-glucanohydrolase activity; preferably endomutanase activity or endodextranase activity; and
iv. optionally one or more acceptors;

b. combining the set of reaction components under suitable aqueous reaction conditions whereby a product comprising a soluble α-glucan fiber composition is produced;

c. optionally isolating the soluble α-glucan fiber composition from the product of step (b); and

d. optionally concentrating the soluble α-glucan fiber composition.

In another embodiment to any of the above embodiments, the at least one polypeptide having glucosyltransferase activity and the at least one polypeptide having α-glucanohydrolase activity are concomitantly present during the reaction.

In another embodiment to any of the above embodiments, the endomutanase comprises an amino acid sequence having at least 90% identity to SEQ ID NO: 4, 6, 9 or 11.

In another embodiment to any of the above embodiments, the at least one polypeptide having α-glucanohydrolase activity is an endodextranase from L from Chaetomium erraticum.

In another embodiment to any of the above embodiments, the ratio of glucosyltransferase activity to α-glucanohydrolase activity is 0.01:1 to 1:0.01.

In another embodiment, a method to produce the present α-glucan fiber composition is provided comprising:

a. providing a set of reaction components comprising:
i. sucrose;
ii. at least one polypeptide having glucosyltransferase activity comprising an amino acid sequence having at least 90% identity to at least one sequence selected from SEQ ID NOs: 13, 16, 17, 19, 28, 30, 32, 34, 36, 38, 40, 42, 44, 46, 48, 50, 52, 54, 56, 58, 60, and 62; and
iii. optionally one or more acceptors;

b. combining the set of reaction components under suitable aqueous reaction conditions to form a single reaction mixture, whereby a product mixture comprising glucose oligomers is formed;

c. optionally isolating the present soluble α-glucan fiber composition from the product mixture comprising glucose oligomers; and

d. optionally concentrating the soluble α-glucan fiber composition.

A composition or method according to any of the above embodiments wherein the carbohydrate composition comprises: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hem icellulose, fructose oligomer syrup, an isomaltooligosaccharide, a filler, an excipient, a binder, or any combination thereof.

A composition or method according to any of the above embodiments wherein the carbohydrate composition is in the form of a liquid, a syrup, a powder, granules, shaped spheres, shaped sticks, shaped plates, shaped cubes, tablets, powders, capsules, sachets, or any combination thereof.

A composition or method according to any of the above embodiments where the food product is
a. a bakery product selected from the group consisting of cakes, brownies, cookies, cookie crisps, muffins, breads, and sweet doughs, extruded cereal pieces, and coated cereal pieces;
b. a dairy product selected from the group consisting of yogurt, yogurt drinks, milk drinks, flavored milks, smoothies, ice cream, shakes, cottage cheese, cottage cheese dressing, quarg, and whipped mousse-type products;
c. confections selected from the group consisting of hard candies, fondants, nougats and marshmallows, gelatin jelly candies, gummies, jellies, chocolate, licorice, chewing gum, caramels, toffees, chews, mints, tableted confections, and fruit snacks;
d. beverages selected from the group consisting of carbonated beverages, fruit juices, concentrated juice mixes, clear waters, and beverage dry mixes;
e. high solids fillings for snack bars, toaster pastries, donuts, or cookies;
f. extruded and sheeted snacks selected from the group consisting of puffed snacks, crackers, tortilla chips, and corn chips;
g. snack bars, nutrition bars, granola bars, protein bars, and cereal bars;
h. cheeses, cheese sauces, and other edible cheese products;
i. edible films;
j. water soluble soups, syrups, sauces, dressings, or coffee creamers; or
k. dietary supplements; preferably in the form of tablets, powders, capsules or sachets.

A composition comprising 0.01 to 99 wt % (dry solids basis) of the present soluble α-glucan fiber composition and: a synbiotic, a peptide, a peptide hydrolysate, a protein, a protein hydrolysate, a soy protein, a dairy protein, an amino acid, a polyol, a polyphenol, a vitamin, a mineral, an herbal, an herbal extract, a fatty acid, a polyunsaturated fatty acid (PUFAs), a phytosteroid, betaine, a carotenoid, a digestive enzyme, a probiotic organism or any combination thereof.

A method according to any of the above methods wherein the isolating step comprises at least one of centrifugation, filtration, fractionation, chromatographic separation, dialysis, evaporation, dilution or any combination thereof.

A method according to any of the above methods wherein the sucrose concentration in the single reaction mixture is initially at least 50 g/L upon when the set of reaction components are combined.

A method according to any of the above methods wherein the ratio of glucosyltransferase activity to α-glucanohydrolase activity ranges from 0.01:1 to 1:0.01.

A method according to any of the above methods wherein the suitable aqueous reaction conditions comprise a reaction temperature between 0° C. and 45° C.

A method according to any of the above methods wherein the suitable aqueous reaction conditions comprise a pH range of 3 to 8, preferably 4 to 8.

A method according to any of the above methods wherein the suitable aqueous reaction conditions comprise including a buffer selected from the group consisting of phosphate, pyrophosphate, bicarbonate, acetate, and citrate

A method according to any of the above methods wherein said at least one glucosyltransferase is selected from the group consisting of SEQ ID NOs: 1, 3, 13, 16, 17, 19, 28, 30, 32, 34, 36, 38, 40, 42, 44, 46, 48, 50, 52, 54, 56, 58, 60, 62 and any combination thereof.

A method according to any of the above embodiments wherein said at least one α-glucanohydrolase is selected from the group consisting of SEQ ID NOs 4, 6, 9, 11 and any combination thereof.

A method according to any of the above embodiments wherein said at least one glucosyltransferase and said at least one α-glucanohydrolase is selected from the combinations of glucosyltransferase GTF0544 (SEQ ID NO: 1, 3 or a combination thereof) and mutanase MUT3264 (SEQ ID NOs: 4, 6, 9 or a combination thereof).

A product produced by any of the above process embodiments; preferably wherein the product produced is the soluble α-glucan fiber composition of the first embodiment.

### EXAMPLES

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “sec” or “s” means second(s), “ms” mean milliseconds, “min” means minute(s), “h” or “hr” means hour(s), “μL” means microliter(s), “mL” means milliliter(s), “L” means liter(s); “mL/min” is milliliters per minute; “μg/mL” is microgram(s) per milliliter(s); “LB” is Luria broth; “μm” is micrometers, “nm” is nanometers; “OD” is optical density; “IPTG” is isopropyl-β-D-thio-galactoside; “g” is gravitational force; “mM” is millimolar; “SDS-PAGE” is sodium dodecyl sulfate polyacrylamide; “mg/mL” is milligrams per milliliters; “N” is normal; “w/v” is weight for volume; “DTT” is dithiothreitol; “BCA” is bicinchoninic acid; “DMAc” is N, N′-dimethyl acetamide; “LiCl” is Lithium chloride′ “NMR” is nuclear magnetic resonance; “DMSO” is dim ethylsulfoxide; “SEC” is size exclusion chromatography; “GI” or “gi” means GenInfo Identifier, a system used by GENBANK® and other sequence databases to uniquely identify polynucleotide and/or polypeptide sequences within the respective databases; “DPx” means glucan degree of polymerization having “x” units in length; “ATCC” means American Type Culture Collection (Manassas, Va.), “DSMZ” and “DSM” will refer to Leibniz Institute DSMZ-German Collection of Microorganisms and Cell Cultures, (Braunschweig, Germany); “EELA” is the Finish Food Safety Authority (Helsinki, Finland;)“CCUG” refer to the Culture Collection, University of Goteborg, Sweden; “Suc.” means sucrose; “Gluc.” means glucose; “Fruc.” means fructose; “Leuc.” means leucrose; and “Rxn” means reaction.

### General Methods

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N Y (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., (American Society for Microbiology Press, Washington, D.C. (1994)), Biotechnology: A Textbook of Industrial Microbiology by Wulf Crueger and Anneliese Crueger (authors), Second Edition, (Sinauer Associates, Inc., Sunderland, Mass. (1990)), and Manual of Industrial Microbiology and Biotechnology, Third Edition, Richard H. Baltz, Arnold L. Demain, and Julian E. Davis (Editors), (American Society of Microbiology Press, Washington, D.C. (2010).

All reagents, restriction enzymes and materials used for the growth and maintenance of bacterial cells were obtained from BD Diagnostic Systems (Sparks, Md.), Invitrogen/Life Technologies Corp. (Carlsbad, Calif.), Life Technologies (Rockville, Md.), QIAGEN (Valencia, Calif.), Sigma-Aldrich Chemical Company (St. Louis, Mo.) or Pierce Chemical Co. (A division of Thermo Fisher Scientific Inc., Rockford, Ill.) unless otherwise specified. IPTG, (cat#16758) and triphenyltetrazolium chloride were obtained from the Sigma Co., (St. Louis, Mo.). Bellco spin flask was from the Bellco Co., (Vineland, N.J.). LB medium was from Becton, Dickinson and Company (Franklin Lakes, N.J.). BCA protein assay was from Sigma-Aldrich (St Louis, Mo.).

Growth of Recombinant E. coli Strains for Production of GTF Enzymes

Escherichia coli strains expressing a functional GTF enzyme were grown in shake flask using LB medium with ampicillin (100 μg/mL) at 37° C. and 220 rpm to OD_{600 nm}=0.4-0.5, at which time isopropyl-β-D-thio-galactoside (IPTG) was added to a final concentration of 0.5 mM and incubation continued for 2-4 hr at 37° C. Cells were harvested by centrifugation at 5,000×g for 15 min and resuspended (20%-25% wet cell weight/v) in 50 mM phosphate buffer pH 7.0). Resuspended cells were passed through a French Pressure Cell (SLM Instruments, Rochester, N.Y.) twice to ensure >95% cell lysis. Cell lysate was centrifuged for 30 min at 12,000×g and 4° C. The resulting supernatant (cell extract) was analyzed by the BCA protein assay and SDS-PAGE to confirm expression of the GTF enzyme, and the cell extract was stored at −80° C.

pHYT Vector

The pHYT vector backbone is a replicative Bacillus subtilis expression plasmid containing the Bacillus subtilis aprE promoter. It was derived from the Escherichia coli-Bacillus subtilis shuttle vector pHY320PLK (GENBANK® Accession No. D00946 and is commercially available from Takara Bio Inc. (Otsu, Japan)). The replication origin for Escherichia coli and ampicillin resistance gene are from pACYC177 (GENBANK® X06402 and is commercially available from New England Biolabs Inc., Ipswich, Mass.). The replication origin for Bacillus subtilis and tetracycline resistance gene were from pAMalpha-1 (Francia et al., J Bacteriol. 2002 September; 184(18):5187-93).

To construct pHYT, a terminator sequence: 5′-ATAAAAAACGCTCGGTTGCCGCCGGGCGTTTTTTAT-3′ (SEQ ID NO: 24) from phage lambda was inserted after the tetracycline resistance gene. The entire expression cassette (EcoRI-BamHI fragment) containing the aprE promoter -AprE signal peptide sequence-coding sequence encoding the enzyme of interest (e.g., coding sequences for various GTFs)-BPN′ terminator was cloned into the EcoRI and HindIII sites of pHYT using a BamHI-HindIII linker that destroyed the HindIII site. The linker sequence is 5′-GGATCCTGACTGCCTGAGCTT-3′ (SEQ ID NO: 25). The aprE promoter and AprE signal peptide sequence (SEQ ID NO: 7) are native to Bacillus subtilis. The BPN′ terminator is from subtilisin of Bacillus amyloliquefaciens. In the case when native signal peptide was used, the AprE signal peptide was replaced with the native signal peptide of the expressed gene.

Biolistic transformation of T. reesei A Trichoderma reesei spore suspension was spread onto the center ˜6 cm diameter of an acetamidase transformation plate (150 μL of a 5×10⁷-5×10⁸ spore/mL suspension). The plate was then air dried in a biological hood. The stopping screens (BioRad 165-2336) and the macrocarrier holders (BioRad 1652322) were soaked in 70% ethanol and air dried. DRIERITE® desiccant (calcium sulfate desiccant; W.A. Hammond DRIERITE® Company, Xenia, Ohio) was placed in small Petri dishes (6 cm Pyrex) and overlaid with Whatman filter paper (GE Healthcare Bio-Sciences, Pittsburgh, Pa.). The macrocarrier holder containing the macrocarrier (BioRad 165-2335; Bio-Rad Laboratories, Hercules, Calif.) was placed flatly on top of the filter paper and the Petri dish lid replaced. A tungsten particle suspension was prepared by adding 60 mg tungsten M-10 particles (microcarrier, 0.7 micron, BioRad #1652266, Bio-Rad Laboratories) to an Eppendorf tube. Ethanol (1 mL) (100%) was added. The tungsten was vortexed in the ethanol solution and allowed to soak for 15 minutes. The Eppendorf tube was microfuged briefly at maximum speed to pellet the tungsten. The ethanol was decanted and washed three times with sterile distilled water. After the water wash was decanted the third time, the tungsten was resuspended in 1 mL of sterile 50% glycerol. The transformation reaction was prepared by adding 25 μL suspended tungsten to a 1.5 mL-Eppendorf tube for each transformation. Subsequent additions were made in order, 2 μL DNA pTrex3 expression vector (SEQ ID NO: 12; see U.S. Pat. No. 6,426,410), 25 μL 2.5M CaCl2, 10 μL 0.1M sperm idine. The reaction was vortexed continuously for 5-10 minutes, keeping the tungsten suspended. The Eppendorf tube was then microfuged briefly and decanted. The tungsten pellet was washed with 200 μL of 70% ethanol, microfuged briefly to pellet and decanted. The pellet was washed with 200 μL of 100% ethanol, microfuged briefly to pellet, and decanted. The tungsten pellet was resuspended in 24 μL 100% ethanol. The Eppendorf tube was placed in an ultrasonic water bath for 15 seconds and 8 μL aliquots were transferred onto the center of the desiccated macrocarriers. The macrocarriers were left to dry in the desiccated Petri dishes.

A Helium tank was turned on to 1500 psi (˜10.3 MPa). 1100 psi (˜7.58 MPa) rupture discs (BioRad 165-2329) were used in the Model PDS-1000/He™ BIOLISTIC® Particle Delivery System (BioRad). When the tungsten solution was dry, a stopping screen and the macrocarrier holder were inserted into the PDS-1000. An acetamidase plate, containing the target T. reesei spores, was placed 6 cm below the stopping screen. A vacuum of 29 inches Hg (˜98.2 kPa) was pulled on the chamber and held. The He BIOLISTIC® Particle Delivery System was fired. The chamber was vented and the acetamidase plate removed for incubation at 28° C. until colonies appeared (5 days).

Modified amdS Biolistic agar (MABA) per liter
Part I, make in 500 mL distilled water (dH₂O)
1000× salts 1 mL
Noble agar 20 g
pH to 6.0, autoclave
Part II, make in 500 mL dH₂O

### Acetamide 0.6 g

### CsCl 1.68 g

### Glucose 20 g

KH₂PO₄ 15 g
MgSO₄.7H₂O 0.6 g
CaCl₂.2H₂O 0.6 g
pH to 4.5, 0.2 micron filter sterilize; leave in 50° C. oven to warm, add to agar, mix, pour plates. Stored at room temperature (˜21° C.)
1000× Salts per liter
FeSO₄.7H₂O 5 g
MnSO₄.H₂O 1.6 g
ZnSO₄.7H₂O 1.4 g
CoCl₂.6H₂O 1 g
Bring up to 1 L dH₂O.
0.2 micron filter sterilize

### Determination of the Glucosyltransferase Activity

Glucosyltransferase activity assay was performed by incubating 1-10% (v/v) crude protein extract containing GTF enzyme with 200 g/L sucrose in 25 mM or 50 mM sodium acetate buffer at pH 5.5 in the presence or absence of 25 g/L dextran (MW ˜1500, Sigma-Aldrich, Cat.#31394) at 37° C. and 125 rpm orbital shaking. One aliquot of reaction mixture was withdrawn at 1 h, 2 h and 3 h and heated at 90° C. for 5 min to inactivate the GTF. The insoluble material was removed by centrifugation at 13,000×g for 5 min, followed by filtration through 0.2 μm RC (regenerated cellulose) membrane. The resulting filtrate was analyzed by HPLC using two Aminex HPX-87C columns series at 85° C. (Bio-Rad, Hercules, Calif.) to quantify sucrose concentration. The sucrose concentration at each time point was plotted against the reaction time and the initial reaction rate was determined from the slope of the linear plot. One unit of GTF activity was defined as the amount of enzyme needed to consume one micromole of sucrose in one minute under the assay condition.

### Determination of the α-Glucanohydrolase Activity

Insoluble mutan polymers required for determining mutanase activity were prepared using secreted enzymes produced by Streptococcus sobrinus ATCC® 33478™. Specifically, one loop of glycerol stock of S. sobrinus ATCC® 33478™ was streaked on a BHI agar plate (Brain Heart Infusion agar, Teknova, Hollister, Calif.), and the plate was incubated at 37° C. for 2 days; A few colonies were picked using a loop to inoculate 2×100 mL BHI liquid medium in the original medium bottle from Teknova, and the culture was incubated at 37° C., static for 24 h. The resulting cells were removed by centrifugation and the resulting supernatant was filtered through 0.2 μm sterile filter; 2×101 mL of filtrate was collected. To the filtrate was added 2×11.2 mL of 200 g/L sucrose (final sucrose 20 g/L). The reaction was incubated at 37° C., with no agitation for 67 h. The resulting polysaccharide polymers were collected by centrifugation at 5000×g for 10 min. The supernatant was carefully decanted. The insoluble polymers were washed 4 times with 40 mL of sterile water. The resulting mutan polymers were lyophilized for 48 h. Mutan polymer (390 mg) was suspended in 39 mL of sterile water to make suspension of 10 mg/mL. The mutan suspension was homogenized by sonication (40% amplitude until large lumps disappear, ˜10 min in total). The homogenized suspension was aliquoted and stored at 4° C.

A mutanase assay was initiated by incubating an appropriate amount of enzyme with 0.5 mg/mL mutan polymer (prepared as described above) in 25 mM KOAc buffer at pH 5.5 and 37° C. At various time points, an aliquot of reaction mixture was withdrawn and quenched with equal volume of 100 mM glycine buffer (pH 10). The insoluble material in each quenched sample was removed by centrifugation at 14,000×g for 5 min. The reducing ends of oligosaccharide and polysaccharide polymer produced at each time point were quantified by the p-hydroxybenzoic acid hydrazide solution (PAHBAH) assay (Lever M., Anal. Biochem., (1972) 47:273-279) and the initial rate was determined from the slope of the linear plot of the first three or four time points of the time course. The PAHBAH assay was performed by adding 10 μL of reaction sample supernatant to 100 μL of PAHBAH working solution and heated at 95° C. for 5 min. The working solution was prepared by mixing one part of reagent A (0.05 g/mL p-hydroxy benzoic acid hydrazide and 5% by volume of concentrated hydrochloric acid) and four parts of reagent B (0.05 g/mL NaOH, 0.2 g/mL sodium potassium tartrate). The absorption at 410 nm was recorded and the concentration of the reducing ends was calculated by subtracting appropriate background absorption and using a standard curve generated with various concentrations of glucose as standards.

### Determination of Glycosidic Linkages

One-dimensional ¹H NMR data were acquired on a Varian Unity Inova system (Agilent Technologies, Santa Clara, Calif.) operating at 500 MHz using a high sensitivity cryoprobe. Water suppression was obtained by carefully placing the observe transmitter frequency on resonance for the residual water signal in a “presat” experiment, and then using the “tnnoesy” experiment with a full phase cycle (multiple of 32) and a mix time of 10 ms.

Typically, dried samples were taken up in 1.0 mL of D₂O and sonicated for 30 min. From the soluble portion of the sample, 1004 was added to a 5 mm NMR tube along with 3504 D₂O and 1004 of D₂O containing 15.3 mM DSS (4,4-dimethyl-4-silapentane-1-sulfonic acid sodium salt) as internal reference and 0.29% NaN₃ as bactericide. The abundance of each type of anomeric linkage was measured by the integrating the peak area at the corresponding chemical shift. The percentage of each type of anomeric linkage was calculated from the abundance of the particular linkage and the total abundance anomeric linkages from oligosaccharides.

### Methylation Analysis

The distribution of glucosidic linkages in glucans was determined by a well-known technique generally named “methylation analysis,” or “partial methylation analysis” (see: F. A. Pettolino, et al., Nature Protocols, (2012) 7(9):1590-1607). The technique has a number of minor variations but always includes: 1. methylation of all free hydroxyl groups of the glucose units, 2. hydrolysis of the methylated glucan to individual monomer units, 3. reductive ring-opening to eliminate anomers and create methylated glucitols; the anomeric carbon is typically tagged with a deuterium atom to create distinctive mass spectra, 4. acetylation of the free hydroxyl groups (created by hydrolysis and ring opening) to create partially methylated glucitol acetates, also known as partially methylated products, 5. analysis of the resulting partially methylated products by gas chromatography coupled to mass spectrometry and/or flame ionization detection.

The partially methylated products include non-reducing terminal glucose units, linked units and branching points. The individual products are identified by retention time and mass spectrometry. The distribution of the partially-methylated products is the percentage (area %) of each product in the total peak area of all partially methylated products. The gas chromatographic conditions were as follows: RTx-225 column (30 m×250 μm ID×0.1 μm film thickness, Restek Corporation, Bellefonte, Pa., USA), helium carrier gas (0.9 mL/min constant flow rate), oven temperature program starting at 80° C. (hold for 2 min) then 30° C./min to 170° C. (hold for 0 min) then 4° C./min to 240° C. (hold for 25 min), 1 μL injection volume (split 5:1), detection using electron impact mass spectrometry (full scan mode)

### Viscosity Measurement

The viscosity of 12 wt % aqueous solutions of soluble fiber was measured using a TA Instruments AR-G2 controlled-stress rotational rheometer (TA Instruments—Waters, LLC, New Castle, Del.) equipped with a cone and plate geometry. The geometry consists of a 40 mm 2° upper cone and a peltier lower plate, both with smooth surfaces. An environmental chamber equipped with a water-saturated sponge was used to minimize solvent (water) evaporation during the test. The viscosity was measured at 20° C. The peltier was set to the desired temperature and 0.65 mL of sample was loaded onto the plate using an Eppendorf pipette (Eppendorf North America, Hauppauge, N.Y.). The cone was lowered to a gap of 50 μm between the bottom of the cone and the plate. The sample was thermally equilibrated for 3 minutes. A shear rate sweep was performed over a shear rate range of 500-10 s⁻¹. Sample stability was confirmed by running repeat shear rate points at the end of the test.

### Determination of the Concentration of Sucrose, Glucose, Fructose and Leucrose

Sucrose, glucose, fructose, and leucrose were quantitated by HPLC with two tandem Aminex HPX-87C Columns (Bio-Rad, Hercules, Calif.). Chromatographic conditions used were 85° C. at column and detector compartments, 40° C. at sample and injector compartment, flow rate of 0.6 mL/min, and injection volume of 10 μL. Software packages used for data reduction were EMPOWER™ version 3 from Waters (Waters Corp., Milford, Mass.). Calibrations were performed with various concentrations of standards for each individual sugar.

### Determination of the Concentration of Oligosaccharides

Soluble oligosaccharides were quantitated by HPLC with two tandem Aminex HPX-42A columns (Bio-Rad). Chromatographic conditions used were 85° C. column temperature and 40° C. detector temperature, water as mobile phase (flow rate of 0.6 m L/min), and injection volume of 10 μL. Software package used for data reduction was EMPOWER™ version 3 from Waters Corp. Oligosaccharide samples from DP2 to DP7 were obtained from Sigma-Aldrich: maltoheptaose (DP7, Cat.#47872), maltohexanose (DP6, Cat.#47873), maltopentose (DP5, Cat.#47876), maltotetraose (DP4, Cat.#47877), isomaltotriose (DP3, Cat.#47884) and maltose (DP2, Cat.#47288). Calibration was performed for each individual oligosaccharide with various concentrations of the standard.

### Determination of Digestibility

The digestibility test protocol was adapted from the Megazyme Integrated Total Dietary Fiber Assay (AOAC method 2009.01, Ireland). The final enzyme concentrations were kept the same as the AOAC method: 50 Unit/mL of pancreatic α-amylase (PAA), 3.4 Units/mL for amyloglucosidase (AMG). The substrate concentration in each reaction was 25 mg/mL as recommended by the AOAC method. The total volume for each reaction was 1 mL instead of 40 mL as suggested by the original protocol. Every sample was analyzed in duplicate with and without the treatment of the two digestive enzymes. The detailed procedure is described below:

The enzyme stock solution was prepared by dissolving 20 mg of purified porcine pancreatic α-amylase (150,000 Units/g; AOAC Method 2002.01) from the Integrated Total Dietary Fiber Assay Kit in 29 mL of sodium maleate buffer (50 mM, pH 6.0 plus 2 mM CaCl₂) and stir for 5 min, followed by the addition of 60 uL amyloglucosidase solution (AMG, 3300 Units/mL) from the same kit. 0.5 mL of the enzyme stock solution was then mixed with 0.5 mL soluble fiber sample (50 mg/mL) in a glass vial and the digestion reaction mixture was incubated at 37° C. and 150 rpm in orbital motion in a shaking incubator for exactly 16 h. Duplicated reactions were performed in parallel for each fiber sample. The control reactions were performed in duplicate by mixing 0.5 mL maleate buffer (50 mM, pH 6.0 plus 2 mM CaCl₂) and 0.5 mL soluble fiber sample (50 mg/mL) and reaction mixtures was incubated at 37° C. and 150 rpm in orbital motion in a shaking incubator for exactly 16 h. After 16 h, all samples were removed from the incubator and immediately 75 μL of 0.75 M TRIZMA® base solution was added to terminate the reaction. The vials were immediately placed in a heating block at 95-100° C., and incubate for 20 min with occasional shaking (by hand). The total volume of each reaction mixture is 1.075 mL after quenching. The amount of released glucose in each reaction was quantified by HPLC with the Aminex HPX-87C Columns (BioRad) as described in the General Methods. Maltodextrin (DE4-7, Sigma) was used as the positive control for the enzymes. To calculate the digestibility, the following formula was used:

Digestibility=100%*[amount of glucose (mg) released after treatment with enzyme−amount of glucose (mg) released in the absence of enzyme]/1.1*amount of total fiber (mg)”

### Purification of Soluble Oligosaccharide Fiber

Soluble oligosaccharide fiber present in product mixtures produced by the conversion of sucrose using glucosyltransferase enzymes with or without added mutanases as described in the following examples were purified and isolated by size-exclusion column chromatography (SEC). In a typical procedure, product mixtures were heat-treated at 60° C. to 90° C. for between 15 min and 30 min and then centrifuged at 4000 rpm for 10 min. The resulting supernatant was injected onto an ÄKTAprime purification system (SEC; GE Healthcare Life Sciences) (10 mL-50 mL injection volume) connected to a GE HK 50/60 column packed with 1.1 L of Bio-Gel P2 Gel (Bio-Rad, Fine 45-90 μm) using water as eluent at 0.7 mL/min. The SEC fractions (˜5 mL per tube) were analyzed by HPLC for oligosaccharides using a Bio-Rad HPX-47A column. Fractions containing >DP2 oligosaccharides were combined and the soluble fiber isolated by rotary evaporation of the combined fractions to produce a solution containing between 3% and 6% (w/w) solids, where the resulting solution was lyophilized to produce the soluble fiber as a solid product.

### Pure Culture Growth on Specific Carbon Sources

To test the capability of microorganisms to grow on specific carbon sources (oligosaccharide or polysaccharide soluble fibers), selected microbes were grown in appropriate media free from carbon sources other than the ones under study. Growth was evaluated by regular (every 30 min) measurement of optical density at 600 nm in an anaerobic environment (80% N₂, 10% CO₂, 10% H₂). Growth was expressed as area under the curve and compared to a positive control (glucose) and a negative control (no added carbon source).

Stock solutions of oligosaccharide soluble fibers (10% w/w) were prepared in demineralised water. The solutions were either sterilised by UV radiation or filtration (0.2 μm). Stocks were stored frozen until used. Appropriate carbon source-free medium was prepared from single ingredients. Test organisms were pre-grown anaerobically in the test medium with the standard carbon source. In honeycomb wells, 20 μL of stock solution was pipetted and 180 μL carbon source-free medium with 1% test microbe was added. As positive control, glucose was used as carbon source, and as negative control, no carbon source was used. To confirm sterility of the stock solutions, uninocculated wells were used. At least three parallel wells were used per run.

The honeycomb plates were placed in a Bioscreen and growth was determined by measuring absorbance at 600 nm. Measurements were taken every 30 min and before measurements, the plates were shaken to assure an even suspension of the microbes. Growth was followed for 24 h. Results were calculated as area under the curve (i.e., OD₆₀₀/24 h). Organisms tested (and their respective growth medium) were: Clostridium perfringens ATCC® 3626™ (anaerobic Reinforced Clostridial Medium (from Oxoid Microbiology Products, ThermoScientific) without glucose), Clostridium difficile DSM 1296 (Deutsche Sammlung von Mikroorganismen and Zellkulturen DSMZ, Braunschweig, Germany) (anaerobic Reinforced Clostridial Medium (from Oxoid Microbiology Products, Thermo Fisher Scientific Inc., Waltham, Mass.) without glucose), Escherichia coli ATCC® 11775™ (anaerobic Trypticase Soy Broth without glucose), Salmonella typhimurium EELA (available from DSMZ, Brauchschweig, Germany) (anaerobic Trypticase Soy Broth without glucose), Lactobacillus acidophilus NCFM 145 (anaerobic de Man, Rogosa and Sharpe Medium (from DSMZ) without glucose), Bifidobacterium animalis subsp. Lactis Bi-07 (anaerobic Deutsche Sammlung vom Mikroorgnismen and Zellkulturen medium 58 (from DSMZ), without glucose).

### In Vitro Gas Production

To measure the formation of gas by the intestinal microbiota, a pre-conditioned faecal slurry was incubated with test prebiotic (oligosaccharide or polysaccharide soluble fibers) and the volume of gas formed was measured. Fresh faecal material was pre-conditioned by dilution with 3 parts (w/v) of anaerobic simulator medium, stirring for 1 h under anaerobic conditions and filtering through 0.3-mm metal mesh after which it was incubated anaerobically for 24 h at 37° C.

The simulator medium used was composed as described by G. T. Macfarlane et al. (Microb. Ecol. 35(2):180-7 (1998)) containing the following constituents (g/L) in distilled water: starch (BDH Ltd.), 5.0; peptone, 0.05; tryptone, 5.0; yeast extract, 5.0; NaCl, 4.5; KCl, 4.5; mucin (porcine gastric type III), 4.0; casein (BDH Ltd.), 3.0; pectin (citrus), 2.0; xylan (oatspelt), 2.0; arabinogalactan (larch wood), 2.0; NaHCO₃, 1.5; MgSO₄, 1.25; guar gum, 1.0; inulin, 1.0; cysteine, 0.8; KH₂PO₄, 0.5; K₂HPO₄, 0.5; bile salts No. 3, 0.4; CaCl₂×6 H₂O, 0.15; FeSO₄×7 H₂O, 0.005; hemin, 0.05; and Tween 80, 1.0; cysteine hydrochloride, 6.3; Na₂S×9H₂O, and 0.1% resazurin as an indication of sustained anaerobic conditions. The simulation medium was filtered through 0.3 mm metal mesh and divided into sealed serum bottles.

Test prebiotics were added from 10% (w/w) stock solutions to a final concentration of 1° A. The incubation was performed at 37° C. while maintaining anaerobic conditions. Gas production due to microbial activity was measured manually after 24 h incubation using a scaled, airtight glass syringe, thereby also releasing the overpressure from the simulation unit.

### Example 1

### Production of GTF-B GI:290580544 in E. coli TOP10

A polynucleotide encoding a truncated version of a glucosyltransferase enzyme identified in GENBANK® as GI:290580544 (SEQ ID NO: 1; Gtf-B from Streptococcus mutans NN2025) was synthesized using codons optimized for expression in E. coli (DNA 2.0). The nucleic acid product (SEQ ID NO: 2) encoding protein “GTF0544” (SEQ ID NO: 3) was subcloned into PJEXPRESS404® to generate the plasmid identified as pMP67. The plasmid pMP67 was used to transform E. coli TOP10 to generate the strain identified as TOP10/pMP67. Growth of the E. coli strain TOP10/pMP67 expressing the Gtf-B enzyme “GTF0544” (SEQ ID NO: 3) and determination of the GTF0544 activity followed the methods described above.

### Example 2

### Production of Mutanase MUT3264 GI: 257153264 in E. coli BL21(DE3)

A gene encoding mutanase from Paenibacillus Humicus NA1123 identified in GENBANK® as GI:257153264 (SEQ ID NO: 4) was synthesized by GenScript (GenScript USA Inc., Piscataway, N.J.). The nucleotide sequence (SEQ ID NO: 5) encoding protein sequence (“MUT3264”; SEQ ID NO: 6) was subcloned into pET24a (Novagen; Merck KGaA, Darmstadt, Germany). The resulting plasmid was transformed into E. coli BL21(DE3) (Invitrogen) to generate the strain identified as SGZY6. The strain was grown at 37° C. with shaking at 220 rpm to OD₆₀₀ of ˜0.7, then the temperature was lowered to 18° C. and IPTG was added to a final concentration of 0.4 mM. The culture was grown overnight before harvest by centrifugation at 4000 g. The cell pellet from 600 mL of culture was suspended in 22 mL 50 mM KPi buffer, pH 7.0. Cells were disrupted by French Cell Press (2 passages @ 15,000 psi (103.4 MPa)); cell debris was removed by centrifugation (SORVALL™ SS34 rotor, @13,000 rpm; Thermo Fisher Scientific, Inc., Waltham, Mass.) for 40 min. The supernatant was analyzed by SDS-PAGE to confirm the expression of the “mut3264” mutanase and the crude extract was used for activity assay. A control strain without the mutanase gene was created by transforming E. coli BL21(DE3) cells with the pET24a vector.

### Example 3

### Production of Mutanase MUT3264 GI: 257153264 in B. subtilis Strain BG6006 Strain SG1021-1

SG1021-1 is a Bacillus subtilis mutanase expression strain that expresses the mutanase from Paenibacillus humicus NA1123 isolated from fermented soy bean natto. For recombinant expression in B. subtilis, the native signal peptide was replaced with a Bacillus AprE signal peptide (GENBANK® Accession No. AFG28208; SEQ ID NO: 7). The polynucleotide encoding MUT3264 (SEQ ID NO: 8) was operably linked downstream of an AprE signal peptide (SEQ ID NO: 7) encoding Bacillus expressed MUT3264 provided as SEQ ID NO: 9. A C-terminal lysine was deleted to provide a stop codon prior to a sequence encoding a poly histidine tag.

The B. subtilis host BG6006 strain contains 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB). The wild type mut3264 (as found under GENBANK® GI: 257153264) has 1146 amino acids with the N terminal 33 amino acids deduced as the native signal peptide by the SignalP 4.0 program (Nordahl et al., (2011) Nature Methods, 8:785-786). The mature mut3264 without the native signal peptide was synthesized by GenScript and cloned into the NheI and HindIII sites of the replicative Bacillus expression pHYT vector under the aprE promoter and fused with the B. subtilis AprE signal peptide (SEQ ID NO: 7) on the vector. The construct was first transformed into E. coli DH10B and selected on LB with ampicillin (100 μg/mL) plates. The confirmed construct pDCQ921 was then transformed into B. subtilis BG6006 and selected on the LB plates with tetracycline (12.5 μg/mL). The resulting B. subtilis expression strain SG1021 was purified and a single colony isolate, SG1021-1, was used as the source of the mutanase mut3264. SG1021-1 strain was first grown in LB containing 10 μg/mL tetracycline, and then sub-cultured into GrantsII medium containing 12.5 μg/mL tetracycline and grown at 37° C. for 2-3 days. The cultures were spun at 15,000 g for 30 min at 4° C. and the supernatant filtered through a 0.22 μm filter. The filtered supernatant containing MUT3264 was aliquoted and frozen at −80° C.

### Example 4

### Production of Mutanase MUT3325 GI: 212533325

A gene encoding the Penicillium marneffei ATCC® 18224™ mutanase identified in GENBANK® as GI:212533325 was synthesized by GenScript (Piscataway, N.J.). The nucleotide sequence (SEQ ID NO: 10) encoding protein sequence (MUT3325; SEQ ID NO: 11) was subcloned into plasmid pTrex3 (SEQ ID NO: 12) at SacII and AscI restriction sites, a vector designed to express the gene of interest in Trichoderma reesei, under control of CBHI promoter and terminator, with Aspergillus niger acetamidase for selection. The resulting plasmid was transformed into T. reesei by biolistic injection as described in the general method section, above. The detailed method of biolistic transformation is described in International PCT Patent Application Publication WO2009/126773 A1. A 1 cm² agar plug with spores from a stable clone TRM05-3 was used to inoculate the production media (described below). The culture was grown in the shake flasks for 4-5 days at 28° C. and 220 rpm. To harvest the secreted proteins, the cell mass was first removed by centrifugation at 4000 g for 10 min and the supernatant was filtered through 0.2 μM sterile filters. The expression of mutanase MUT3325 was confirmed by SDS-PAGE.

The production media component is listed below.

### NREL-Trich Lactose Defined

| Formula | Amount | Units |
|---|---|---|
| ammonium sulfate | 5 | g |
| PIPPS | 33 | g |
| BD Bacto casamino acid | 9 | g |
| KH₂PO₄ | 4.5 | g |
| CaCl₂•2H₂O | 1.32 | g |
| MgSO₄•7H₂O | 1 | g |
| T. reesei trace elements | 2.5 | mL |
| NaOH pellet | 4.25 | g |
| Adjust pH to 5.5 with 50% | | |
| NaOH | | |
| Bring volume to | 920 | mL |
| Add to each aliquot: | 5 | Drops |
| Foamblast | | |
| Autoclave, then add | 80 | mL |
| 20% lactose filter sterilized | | |

T. reesei Trace Elements

| Formula | Amount | Units |
|---|---|---|
| citric acid•H₂O | 191.41 | g |
| FeSO₄•7H₂O | 200 | g |
| ZnSO₄•7H₂O | 16 | g |
| CuSO₄•5H₂O | 3.2 | g |
| MnSO₄•H₂O | 1.4 | g |
| H₃BO₃ (boric acid) | 0.8 | g |
| Bring volume to | 1 | L |

### Example 5

### Production of MUT3325 by Fermentation

Fermentation seed culture was prepared by inoculating 0.5 L of minimal medium in a 2-L baffled flask with 1.0 mL frozen spore suspension of the MUT3325 expression strain TRM05-3 (Example 4) (The minimal medium was composed of 5 g/L ammonium sulfate, 4.5 g/L potassium phosphate monobasic, 1.0 g/L magnesium sulfate heptahydrate, 14.4 g/L citric acid anhydrous, 1 g/L calcium chloride dihydrate, 25 g/L glucose and trace elements including 0.4375 g/L citric acid, 0.5 g/L ferrous sulfate heptahydrate, 0.04 g/L zinc sulfate heptahydrate, 0.008 g/L cupric sulfate pentahydrate, 0.0035 g/L manganese sulfate monohydrate and 0.002 g/L boric acid. The pH was 5.5.). The culture was grown at 32° C. and 170 rpm for 48 hours before transferred to 8 L of the production medium in a 14-L fermentor. The production medium was composed of 75 g/L glucose, 4.5 g/L potassium phosphate monobasic, 0.6 g/L calcium chloride dehydrate, 1.0 g/L magnesium sulfate heptahydrate, 7.0 g/L ammonium sulfate, 0.5 g/L citric acid anhydrous, 0.5 g/L ferrous sulfate heptahydrate, 0.04 g/L zinc sulfate heptahydrate, 0.00175 g/L cupric sulfate pentahydrate, 0.0035 g/L manganese sulfate monohydrate, 0.002 g/L boric acid and 0.3 mL/L foam blast 882.

The fermentation was first run with batch growth on glucose at 34° C., 500 rpm for 24 h. At the end of 24 h, the temperature was lowered to 28° C. and agitation speed was increased to 1000 rpm. The fermentor was then fed with a mixture of glucose and sophorose (62% w/w) at specific feed rate of 0.030 g glucose-sophorose solids/g biomass/hr. At the end of run, the biomass was removed by centrifugation and the supernatant containing the mutanase was concentrated about 10-fold by ultrafiltration using 10-kD Molecular Weight Cut-Off ultrafiltration cartridge (UFP-10-E-35; GEHealthcare, Little Chalfont, Buckinghamshire, UK). The concentrated protein was stored at −80° C.

### Example 6

### Isolation of Soluble Oligosaccharide Fiber Produced by the Combination of GTF-B and MUT3264

A 200-mL reaction containing 100 g/L sucrose, E. coli crude protein extract (10% v/v) containing GTF-B from Streptococcus mutans NN2025 (GI:290580544; Example 1), and E. coli crude protein extract (10% v/v) comprising a mutanase from Paenibacillus humicus (MUT3264, GI:257153264; Example 2) in distilled, deionized H₂O, was stirred at 37° C. for 24 h, then heated to 90° C. for 15 min to inactivate the enzymes. The resulting product mixture was centrifuged and the resulting supernatant analyzed by HPLC for soluble monosaccharides, disaccharides and oligosaccharides, then 132 mL of the supernatant was purified by SEC using BioGel P2 resin (BioRad). The SEC fractions that contained oligosaccharides ≧DP3 were combined and concentrated by rotary evaporation for analysis by HPLC (Table 1).

TABLE 1
Soluble oligosaccharide fiber produced by GTF-B/mut3264 mutanase.
100 g/L sucrose, GTF-B, mut3264, 37° C., 24 h

| | Product | SEC-purified |
|---|---|---|
| | mixture, | product, |
| | g/L | g/L |
| DP7 | 2.8 | 11.7 |
| DP6 | 4.0 | 14.0 |
| DP5 | 4.3 | 13.2 |
| DP4 | 3.5 | 9.4 |
| DP3 | 4.4 | 2.4 |
| DP2 | 9.8 | 0.0 |
| Sucrose | 10.3 | 0.2 |
| Leucrose | 15.6 | 0.0 |
| Glucose | 2.9 | 0.0 |
| Fructose | 41.7 | 0.1 |
| Sum DP2-DP7 | 28.8 | 50.7 |
| Sum DP3-DP7 | 19.0 | 50.7 |

### Example 7

### Production of GTF-C GI:3130088 in E. coli BL21

A gene encoding a truncated version of a glucosyltransferase (gtf) enzyme identified in GENBANK® as GI:3130088 (SEQ ID NO: 13; gtfC from S. mutans MT-4239) was synthesized using codons optimized for expression in E. coli (DNA 2.0, Menlo Park, Calif.). The nucleic acid product encoding a truncated version of the S. mutans GTF0088 glucosyltransferase (SEQ ID NO: 14) was subcloned into PJEXPRESS404® (DNA 2.0, Menlo Park Calif.) to generate the plasmid identified as pMP69 (SEQ ID NO: 15). The plasmid pMP69 was used to transform E. coli BL21 (EMD Millipore, Billerica, Mass.) to generate the strain identified as BL21-GI3130088, producing truncated form of the S. mutans GENBANK® gi:3130088 glucosyltransferase; also referred to herein as “GTF0088” (SEQ ID NO: 16). A single colony from the transformation plate was streaked onto a plate containing LB agar with 100 ug/ml ampicillin and incubated overnight at 37° C. A single colony from the plate was inoculated into LB media containing 100 ug/mL ampicillin and grown at 37° C. with shaking at 220 rpm for 3.5 hours. The culture was diluted 1250 fold into 8 flasks containing 2 L total of LB media with 100 ug/ml ampicillin and grown at 37° C. with shaking at 220 rpm for 4 hours. IPTG was added to a final concentration of 0.5 mM and the cultures were grown overnight before harvesting by centrifugation at 9000×g. The cell pellet was suspended in 50 mM KPi buffer, pH 7.0 at a ratio of 5 ml buffer per gram wet cell weight. Cells were disrupted by French Cell Press (2 passages @ 16,000 psi) and cell debris was removed by centrifugation at 25,000×g. Cell free extract was stored at −80° C.

### Example 8

### Production of S. mutans LJ23 GTF GI:387786207 in E. coli TOP10

The amino acid sequence of the Streptococcus mutans LJ23 glucosyltransferase (gtf) as described in GENBANK® as 387786207 is provided as SEQ ID NO: 17. A coding sequence (SEQ ID NO: 18) encoding a truncated version (SEQ ID NO: 19) of the glucosyltransferase (gtf) enzyme identified in GENBANK® as 387786207 (“GTF6207”) from S. mutans LJ23 was prepared by mutagenesis of the pMP69 plasmid described in Example 7. A 1630 bp DNA fragment encoding a portion of GI:387786207 (SEQ ID NO:20) was ordered from GenScript (Piscataway, N.J.). The resultant plasmid (6207f1 in pUC57) was employed as a template for PCR with primers 8807f1 (5′-AATACAATCAGGTGTATTCGACGGATGC-3′; SEQ ID NO: 21) and 8807r1 (5′-TCCTGATCGCTGTGATACGCTTTGATG-3′; SE Q ID NO: 22). The PCR conditions for amplification were as follows: 1. 95° C. for 2 minutes, 2. 95° C. for 40 seconds, 3. 48° C. for 30 seconds, 4. 72° C. for 1.5 minutes, 5. return to step 2 for 30 cycles, 6. 4° C. indefinitely. The reaction sample contained 0.5 uL of plasmid DNA for 6207f1 in pUC57 (90 ng), 4 uL of a mixture of primers 8807f1 and 8807r1 (40 μmol each), 5 uL of the 10×buffer, 2 uL 10 mM dNTPs mixture, 1 uL of the Pfu Ultra AD (Agilent Technologies, Santa Clara, Calif.) and 37.5 uL distilled water. The PCR product was gel purified with the GFX PCR DNA and Gel Band Purification Kit (GE Healthcare Bio-Sciences Corp., Piscataway, N.J.). The purified product was employed as a megaprimer for mutagenesis of pMP69 with the QuikChange Lightning Site-Directed Mutagenesis Kit (Agilent Technologies, Santa Clara, Calif.). The conditions for the mutagenesis reaction were as follows: 1. 95° C. for 2 minutes, 2. 95° C. for 30 seconds, 3. 60° C. for 30 seconds, 4. 68° C. for 12 minutes, 5. return to step 2 for 18 cycles, 6. 68° C. for 7 minutes, 7. 4° C. indefinitely. The reaction sample contained 1 uL of the pMP69 (50 ng), 17 uL of the PCR product (500 ng), 5 uL of the 10×buffer, 1.5 uL QuikSolution reagent, 1 uL of dNTP mixture, 1 uL of QuikChange Lightning Enzyme and 23.5 uL distilled water. 2 uL of DpnI was added and the mixture was incubated for 1 hr at 37° C. The resultant product was then transformed into ONE SHOT® TOP10 Chemically Competent E. coli (Life Technologies, Grand Island, N.Y.). Colonies from the transformation were grown overnight in LB media containing 100 ug/mL ampicillin and plasmids were isolated with the QIAprep Spin Miniprep Kit (Qiaqen, Valencia, Calif.). Sequence analysis was performed to confirm the presence of the gene encoding gi:387786207. The resultant plasmid p6207-1 (SEQ ID NO:22) was transformed into E. coli BL21 (EMD Millipore, Billerica, Mass.) to generate the strain identified as BL21-6207. A single colony from the plate was inoculated into 5 mL LB media containing 100 ug/mL ampicillin and grown at 37° C. with shaking at 220 rpm for 8 hours. The culture was diluted 200 fold into 4 flasks containing 1 L total of LB media with 100 ug/mL ampicillin and 1 mM IPTG. Cultures were grown at 33° C. overnight before harvesting by centrifugation at 9000×g. The cell pellet was suspended in 50 mM KPi buffer, pH 7.0 at a ratio of 5 mL buffer per gram wet cell weight. Cells were disrupted by French Cell Press (2 passages @ 16,000 psi) and cell debris was removed by centrifugation at 25,000×g. Cell free extract was stored at −80° C.

### Example 9

### Isolation of Soluble Oligosaccharide Fiber Produced by GTF-C GI:3130088

A 600-mL reaction containing 200 g/L sucrose, E. coli concentrated crude protein extract (10.0% v/v) containing GTF GI:3130088 from S. mutans MT-4239 GTF-C (Example 7) in distilled, deionized H₂O, was stirred at 30° C. for 22 h, then heated to 90° C. for 10 min to inactivate the enzyme. The resulting product mixture was centrifuged and the resulting supernatant analyzed by HPLC for soluble monosaccharides, disaccharides and oligosaccharides, then the supernatant was purified by SEC using BioGel P2 resin (BioRad). The SEC fractions that contained oligosaccharides ≧DP3 were combined and concentrated by rotary evaporation for analysis by HPLC (Table 2).

TABLE 2
Soluble oligosaccharide fiber produced by GTF GI:3130088.
200 g/L sucrose, GTF-C, 30° C., 22 h

| | Product | SEC-purified |
|---|---|---|
| | mixture, | product, |
| | g/L | g/L |
| ≧DP8 | 29.2 | 49.3 |
| DP7 | 10.0 | 14.5 |
| DP6 | 9.5 | 11.6 |
| DP5 | 9.0 | 8.6 |
| DP4 | 6.2 | 4.3 |
| DP3 | 4.5 | 2.0 |
| DP2 | 5.0 | 1.0 |
| Sucrose | 0.7 | 0.1 |
| Leucrose | 41.3 | 0.0 |
| Glucose | 8.6 | 0.0 |
| Fructose | 64.3 | 0.2 |
| Sum DP2-≧DP8 | 73.4 | 91.3 |
| Sum DP3-≧DP8 | 68.4 | 90.3 |

### Example 10

### Isolation of Soluble Oligosaccharide Fiber Produced by GTF GI: 387786207

A 600-mL reaction containing 200 g/L sucrose, E. coli concentrated crude protein extract (10.0% v/v) containing GTF6207 (SEQ ID NO: 19) from S. mutans 1123 (Example 8) in distilled, deionized H₂O, was stirred at 37° C. for 72 h, then heated to 90° C. for 10 min to inactivate the enzyme. The resulting product mixture was centrifuged and the resulting supernatant analyzed by HPLC for soluble monosaccharides, disaccharides and oligosaccharides, then 580 mL of the supernatant was purified by SEC using BioGel P2 resin (BioRad). The SEC fractions that contained oligosaccharides ≧DP3 were combined and concentrated by rotary evaporation for analysis by HPLC (Table 3).

TABLE 3
Soluble oligosaccharide fiber produced by GTF GI:387786207.
200 g/L sucrose, GIF GI:387786207, 30° C., 72 h

| | Product | SEC-purified |
|---|---|---|
| | mixture, | product, |
| | g/L | g/L |
| ≧DP8 | 19.2 | 83.2 |
| DP7 | 7.9 | 28.3 |
| DP6 | 8.5 | 26.2 |
| DP5 | 7.4 | 24.8 |
| DP4 | 4.9 | 13.1 |
| DP3 | 3.3 | 5.0 |
| DP2 | 4.2 | 2.0 |
| Sucrose | 36.5 | 0.0 |
| Leucrose | 31.5 | 1.5 |
| Glucose | 6.0 | 0.0 |
| Fructose | 56.5 | 1.3 |
| Sum DP2-≧DP8 | 55.4 | 182.6 |
| Sum DP3-≧DP8 | 51.2 | 180.6 |

### Example 11

### Anomeric Linkage Analysis of Soluble Oligosaccharide Fiber Produced by GTF-C and by GTF-6207

Solutions of chromatographically-purified soluble oligosaccharide fibers prepared as described in Examples 6, 9 and 10 were dried to a constant weight by lyophilization, and the resulting solids analyzed by ¹H NMR spectroscopy and by GC/MS as described in the General Methods section (above). The anomeric linkages for each of these soluble oligosaccharide fiber mixtures are reported in Tables 4 and 5.

TABLE 4
Anomeric linkage analysis of soluble oligosaccharides
by ¹H NMR spectroscopy.

| | | % | % | % | % | % |
|---|---|---|---|---|---|---|
| | | α- | α- | α- | α- | α- |
| Example # | GTF | (1,3) | (1,2) | (1,3,6) | (1,2,6) | (1,6) |
| 6 | GTF0544/MUT3264 | 15 | 0 | 3.4 | 0 | 81.6 |
| 9 | GTF-C GI:3130088 | 7.8 | 0.0 | 1.3 | 0 | 90.9 |
| 10 | GTF GI:387786207 | 6.0 | 1.7 | 1.4 | 0 | 90.9 |

TABLE 5
Anomeric linkage analysis of soluble oligosaccharides by GC/MS.

| | | | | | | | | | | % |
|---|---|---|---|---|---|---|---|---|---|---|
| | | % | % | % | % | % | % | % | % | α-(1,4,6) + |
| Example # | GTF | α-(1,4) | α-(1,3) | α-(1,3,6) | 2,1 Fruc | α-(1,2) | α-(1,6) | α-(1,3,4) | α-(1,2,3) | α-(1,2,6) |
| 6 | GTF0544/MUT3264 | 0.4 | 24.1 | 2.5 | 1.0 | 0.5 | 70.9 | 0.0 | 0.0 | 0.6 |
| 9 | GTF-C GI:3130088 | 0.6 | 14.0 | 1.4 | 1.1 | 0.9 | 80.8 | 0.0 | 0.0 | 1.2 |
| 10 | GTF GI:387786207 | 0.3 | 11.8 | 0.0 | 1.1 | 0.5 | 86.3 | 0.0 | 0.0 | 0.0 |

### Example 12

### Viscosity of Soluble Oligosaccharide Fiber Produced by GTF-C and by GTF-6207

Solutions of chromatographically-purified soluble oligosaccharide fibers prepared as described in Examples 6, 9 and 10 were dried to a constant weight by lyophilization, and the resulting solids were used to prepare a 12 wt % solution of soluble fiber in distilled, deionized water. The viscosity of the soluble fiber solutions (reported in centipoise (cP), where 1 cP=1 millipascal-s (mPa-s)) (Table 6) was measured at 20° C. as described in the General Methods section.

TABLE 6
Viscosity of 12% (w/w) soluble oligosaccharide fiber solutions
measured at 20° C. (ND = not determined).

| Example # | GTF | viscosity (cP) |
|---|---|---|
| 6 | GTF0544/MUT3264 | 6.7 |
| 9 | GTF-C GI:3130088 | 1.8 |
| 10 | GTF GI:387786207 | 1.7 |

### Example 13

### Digestibility of Soluble Oligosaccharide Fiber Produced by GTF-C and by GTF-6207

Solutions of chromatographically-purified soluble oligosaccharide fibers prepared as described in Examples 6, 9 and 10 were dried to a constant weight by lyophilization. The digestibility test protocol was adapted from the Megazyme Integrated Total Dietary Fiber Assay (AOAC method 2009.01, Ireland). The final enzyme concentrations were kept the same as the AOAC method: 50 Unit/mL of pancreatic α-amylase (PAA), 3.4 Units/mL for amyloglucosidase (AMG). The substrate concentration in each reaction was 25 mg/mL as recommended by the AOAC method. The total volume for each reaction was 1 mL. Every sample was analyzed in duplicate with and without the treatment of the two digestive enzymes. The amount of released glucose was quantified by HPLC with the Aminex HPX-87C Columns (BioRad) as described in the General Methods. Maltodextrin (DE4-7, Sigma) was used as the positive control for the enzymes (Table 7).

TABLE 7
Digestibility of soluble oligosaccharide fiber.

| Example # | GTF | Digestibility (%) |
|---|---|---|
| 6 | GTF0544/MUT3264 | 9.0 |
| 9 | GTF-C GI:3130088 | 5.6 |
| 10 | GTF GI:387786207 | 6.9 |

### Example 14

### Molecular Weight of Oligosaccharide Fiber Produced by GTF-C or by the Combination of GTF-B and MUT3264

A solution of chromatographically-purified soluble oligosaccharide fibers prepared as described in Examples 9 and Example 6 were dried to a constant weight by lyophilization, and the resulting solids were analyzed by SEC chromatography for number average molecular weight (Mₙ), weight average molecular weight (M_{w}), peak molecular weight (Mₚ), z-average molecular weight (M_{z}), and polydispersity index (PDI=M_{w}/Mₙ) as described in the General Methods section (Table 8).

TABLE 8
Characterization of soluble oligosaccharide fiber by SEC.

| | | Mₙ | M_{w} | Mₚ | M_{z} | |
|---|---|---|---|---|---|---|
| | GTF or | (Dal- | (Dal- | (Dal- | (Dal- | |
| Example # | GTF/mutanase | tons) | tons) | tons) | tons) | PDI |
| 9 | GTF-C GI:3130088 | 821 | 1265 | 1560 | 1702 | 1.54 |
| 6 | GTF0544/mut3264 | 1314 | 1585 | 1392 | 1996 | 1.21 |

### Example 14A

### Construction of Bacillus Subtilis Strains Expressing Homolog Genes of GTF0088

The amino acid sequence of the GTF0088 enzyme (GI 3130088) was used as a query to search the NR database (non-redundant version of the NCBI protein database) with BLAST. From the BLAST search, over 60 sequences were identified having at least 80% identity over an alignment length of at least 1000 amino acids. These sequences were then aligned using CLUSTALW. Using Discovery Studio, a phylogenetic tree was also generated. The tree had three major branches. More than two dozen of the homologs belonged to the same branch as GTF0088. These sequences have amino acid sequence identities between 91.5%-99.5% in an aligned region of ˜1455 residues, which extends from position 1 to 1455 in GTF0088. One of the homologs, GTF6207, was evaluated as described in Examples 10-13. Ten additional homologs, together with GTF0088 in native codons (Table 9) were synthesized with N terminal variable region truncation by Genscript. The synthetic genes were cloned into the NheI and HindIII sites of the Bacillus subtilis integrative expression plasmid p4JH under the aprE promoter and fused with the B. subtilis AprE signal peptide on the vector. In some cases, they were cloned into the SpeI and HindIII sites of the Bacillus subtilis integrative expression plasmid p4JH under the aprE promoter without a signal peptide. The constructs were first transformed into E. coli DH10B and selected on LB with ampicillin (100 ug/ml) plates. The confirmed constructs expressing the particular GTFs were then transformed into B. subtilis host containing 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB) and selected on the LB plates with chloramphenicol (5 ug/ml). The colonies grown on LB plates with 5 ug/ml chloramphenicol were streaked several times onto LB plates with 25 ug/ml chloramphenicol. The resulted B. subtilis expression strains were grown in LB medium with 5 ug/ml chloramphenicol first and then subcultured into GrantsII medium grown at 30° C. for 2-3 days. The cultures were spun at 15,000 g for 30 min at 4° C. and the supernatants were filtered through 0.22 um filters. The filtered supernatants were aliquoted and frozen at −80° C.

TABLE 9
GTF0088 homologues with N terminal truncation
tested in this application

| | | | DNA | aa |
|---|---|---|---|---|
| | | | seq | seq |
| | % | | SEQ | SEQ |
| GI number | Identity | Source Organism | ID | ID |
| gi\|3130088\| | 100.00 | Streptococcus mutans MT4239 | 26 | 16 |
| gi\|387786207\| | 99.50 | Streptococcus mutans LJ23 | 18 | 19 |
| gi\|440355330\| | 99.45 | Streptococcus mutans UA113 | 27 | 28 |
| gi\|440355318\| | 99.45 | Streptococcus mutans BZ15 | 29 | 30 |
| gi\|440355326\| | 99.29 | Streptococcus mutans Leo | 31 | 32 |
| gi\|440355312\| | 99.21 | Streptococcus mutans Asega | 33 | 34 |
| gi\|440355334\| | 99.13 | Streptococcus mutans UA140 | 35 | 36 |
| gi\|3130095\| | 98.97 | Streptococcus mutans MT4251 | 37 | 38 |
| gi\|3130074\| | 98.82 | Streptococcus mutans MT8148 | 39 | 40 |
| gi\|440355320\| | 98.82 | Streptococcus mutans CH638 | 41 | 42 |
| gi\|3130081\| | 97.58 | Streptococcus mutans MT4245 | 43 | 44 |
| gi\|440355328\| | 97.31 | Streptococcus troglodytae Mark | 45 | 46 |

The supernatants containing the GTF0088 homolog enzymes with N terminal truncation were tested for activity in the sucrose conversion assay. After three days, the samples were analyzed by HPLC. The following table shows that all the N terminal truncated homolog enzymes were active in converting sucrose and the profile of the produced small sugars and oligomers was similar.

TABLE 10
HPLC analysis of sucrose conversion by the GTF0088 homologs.

| | DP8 | | | | | | | | | | | | |
|---|---|---|---|---|---|---|---|---|---|---|---|---|---|
| | & up | | | | | | DP3 | | | | | | Total |
| | est. | DP7 | DP6 | DP5 | DP4 | DP3 | & up | DP2 | Sucrose | Leucrose | Glucose | Frucrose | Sugar |
| gene | (g/L) | (g/L) | (g/L) | (g/L) | (g/L) | (g/L) | (g/L) | (g/L) | (g/L) | (g/L) | (g/L) | (g/L) | (g/L) |
| gtf0074NT | 21.6 | 6.6 | 8.6 | 7.5 | 5.6 | 4.2 | 53.9 | 6.0 | 1.1 | 21.0 | 7.0 | 44.5 | 133.4 |
| gtf0081NT | 29.3 | 5.5 | 5.6 | 5.2 | 4.2 | 3.7 | 53.4 | 6.0 | 1.1 | 21.3 | 6.4 | 45.1 | 133.2 |
| gtf0088NT | 20.9 | 6.7 | 7.7 | 7.6 | 5.5 | 4.0 | 52.5 | 5.2 | 1.2 | 19.2 | 7.1 | 45.5 | 130.7 |
| gtf0095NT | 28.6 | 5.6 | 6.3 | 5.5 | 3.9 | 3.2 | 53.0 | 5.2 | 0.9 | 23.0 | 6.8 | 44.3 | 133.3 |
| gtf5312NT | 24.7 | 7.0 | 7.2 | 7.5 | 5.6 | 3.7 | 55.6 | 5.1 | 1.0 | 18.2 | 6.6 | 46.2 | 132.6 |
| gtf5318NT | 25.9 | 7.2 | 6.7 | 7.2 | 5.0 | 3.7 | 55.6 | 4.9 | 1.0 | 18.6 | 6.4 | 46.3 | 132.8 |
| gtf5320NT | 26.6 | 6.1 | 6.4 | 6.1 | 4.7 | 3.9 | 53.8 | 5.3 | 0.9 | 23.7 | 6.6 | 44.9 | 135.3 |
| gtf5326NT | 28.6 | 7.3 | 6.5 | 6.5 | 4.7 | 3.4 | 57.0 | 5.0 | 0.8 | 19.0 | 6.6 | 46.8 | 135.2 |
| gtf5328NT | 23.7 | 7.1 | 7.1 | 7.1 | 5.5 | 4.2 | 54.7 | 6.1 | 1.1 | 18.2 | 6.7 | 46.9 | 133.7 |
| gtf5330NT | 24.7 | 6.8 | 7.8 | 7.5 | 5.6 | 3.9 | 56.4 | 5.2 | 1.0 | 19.0 | 6.6 | 46.7 | 134.8 |
| gtf5334NT | 13.0 | 6.4 | 8.3 | 8.3 | 7.3 | 4.7 | 48.0 | 6.0 | 1.8 | 18.2 | 6.5 | 47.4 | 127.9 |

### Example 14B

### Construction of Bacillus Subtilis Strains Expressing C Terminal Truncations of GTF0088 Homolog Genes

Glucosyltransferases usually contain an N-terminal variable domain, a middle catalytic domain followed by multiple glucan binding domains at the C terminus. The GTF0088 homologs tested in Example 14A all contained the N terminal variable region truncation. Homologs with additional C terminal truncations of part of the glucan binding domains were also prepared and evaluated. This example describes the construction of Bacillus subtilis strains expressing two of the C terminal truncations of GTF0088 homologs.

The C terminal T1 or T3 truncation was made to the GTF0088, GTF5318, GTF5328 and GTF5330 listed in the table in Example 14A. The nucleotide sequences of these T1 strains are shown in SEQ ID NOs: 47-53 (odd numbers); the amino acid sequences of these T1 strains are shown in SEQ ID NOs: 48-54 (even numbers). The nucleotide sequences of the T3 strains are shown in SEQ ID NOs: 55-61 (odd numbers); the amino acid sequences of the T3 strains are shown in SEQ ID NOs: 56-62 (even numbers). The DNA fragments encoding the T1 or T3 truncation were PCR amplified from the synthetic gene plasmids provided by Genscript and cloned into the SpeI and HindIII sites of the Bacillus subtilis integrative expression plasmid p4JH under the aprE promoter without a signal peptide. The constructs were first transformed into E. coli DH10B and selected on LB with ampicillin (100 ug/ml) plates. The confirmed constructs expressing the particular GTFs were then transformed into B. subtilis host strains containing 9 protease deletions (amyE::xylRPxylAcomK-ermC, degUHy32, oppA, ΔspoIIE3501, ΔaprE, ΔnprE, Δepr, ΔispA, Δbpr, Δvpr, ΔwprA, Δmpr-ybfJ, ΔnprB) and selected on the LB plates with chloramphenicol (5 ug/ml). The colonies grown on LB plates with 5 ug/ml chloramphenicol were streaked several times onto LB plates with 25 ug/ml chloramphenicol. The resulting B. subtilis expression strains were grown first in LB medium with 5 ug/ml chloramphenicol and then subcultured into GrantsII medium grown at 30° C. for 2-3 days. The cultures were spun at 15,000 g for 30 min at 4° C. and the supernatants were filtered through 0.22 um filters. The filtered supernatants were aliquoted and frozen at −80° C.

### Example 14C

### Isolation of Soluble Oligosaccharide Fiber Produced by the C-Terminal Truncated GTF0088T1

A 250 mL reaction containing 450 g/L sucrose and B. subtilis crude protein extract (5% v/v) containing a version of GTF0088 from Streptococcus mutans MT4239 (GI: 3130088; Example 14A) having additional C terminal truncations of part of the glucan binding domains (GTF0088-T1, Example 14B) in distilled, deionized H₂O, was stirred at pH 5.5 and 47° C. for 22 h, then heated to 90° C. for 30 min to inactivate the enzymes. The resulting product mixture was centrifuged and the resulting supernatant analyzed by HPLC for soluble monosaccharides, disaccharides and oligosaccharides (Table 11), then the oligosaccharides were isolated from the supernatant by SEC at 40° C. using Diaion UBK 530 (Na⁺ form) resin (Mitsubishi). The SEC fractions that contained oligosaccharides ≧DP3 were combined and concentrated by rotary evaporation for analysis by HPLC (Table 11). The combined SEC fractions were diluted to 5 wt % dry solids (DS) and freeze-dried to produce the fiber as a dry solid.

TABLE 11
Soluble oligosaccharide fiber produced by GTF0088-T1.
450 g/L sucrose, GTF0088-T1, 47° C., 22 h

| | Product | SEC-purified | SEC-purified |
|---|---|---|---|
| | mixture, | product, | product |
| | g/L | g/L | % (wt/wt DS) |
| DP8+ | 74.8 | 47.3 | 44.8 |
| DP7 | 27.1 | 16.4 | 15.5 |
| DP6 | 28.2 | 13.8 | 13.1 |
| DP5 | 26.4 | 12.8 | 12.1 |
| DP4 | 18.5 | 7.2 | 6.8 |
| DP3 | 13.8 | 4.5 | 4.3 |
| DP2 | 16.8 | 2.3 | 2.2 |
| Sucrose | 5.5 | 1.1 | 1.1 |
| Leucrose | 82.4 | 0.2 | 0.2 |
| Glucose | 9.4 | 0.0 | 0.0 |
| Fructose | 156.7 | 0.0 | 0.0 |
| Sum DP2-DP8+ | 205.6 | 104.3 | 98.7 |
| Sum DP3-DP8+ | 188.8 | 102.0 | 96.5 |

### Example 14D

### Isolation of Soluble Oligosaccharide Fiber Produced by the C-Terminal Truncated GTF5318-T1

A 250 mL reaction containing 450 g/L sucrose and B. subtilis crude protein extract (5% v/v) containing a version of GTF5318 from Streptococcus mutans BZ15 (GI: 440355318; Example 14A) having additional C terminal truncations of part of the glucan binding domains (GTF5318-T1, Examples 14A and 14B) in distilled, deionized H₂O, was stirred at pH 5.5 and 47° C. for 4 h, then heated to 90° C. for 30 min to inactivate the enzymes. The resulting product mixture was centrifuged and the resulting supernatant analyzed by HPLC for soluble monosaccharides, disaccharides and oligosaccharides (Table 12), then the oligosaccharides were isolated from the supernatant by SEC at 40° C. using Diaion UBK 530 (Na⁺ form) resin (Mitsubishi). The SEC fractions that contained oligosaccharides ≧DP3 were combined and concentrated by rotary evaporation for analysis by HPLC (Table 12). The combined SEC fractions were diluted to 5 wt % dry solids (DS) and freeze-dried to produce the fiber as a dry solid.

TABLE 12
Soluble oligosaccharide fiber produced by GTF5318-T1.
450 g/L sucrose, GTF5318-T1, 47° C., 4 h

| | Product | SEC-purified | SEC-purified |
|---|---|---|---|
| | mixture, | product, | product |
| | g/L | g/L | % (wt/wt DS) |
| DP8+ | 111.2 | 75.6 | 62.7 |
| DP7 | 19.9 | 13.0 | 10.8 |
| DP6 | 19.5 | 11.6 | 9.6 |
| DP5 | 18.2 | 8.2 | 6.8 |
| DP4 | 14.0 | 5.8 | 4.8 |
| DP3 | 10.7 | 3.6 | 3.0 |
| DP2 | 14.8 | 2.4 | 2.0 |
| Sucrose | 6.4 | 0.0 | 0.0 |
| Leucrose | 82.9 | 0.4 | 0.3 |
| Glucose | 7.7 | 0.0 | 0.0 |
| Fructose | 166.6 | 0.0 | 0.0 |
| Sum DP2-DP8+ | 208.3 | 120.3 | 99.7 |
| Sum DP3-DP8+ | 193.5 | 117.9 | 97.7 |

### Example 14E

### Isolation of Soluble Oligosaccharide Fiber Produced by the C-Terminal Truncated GTF5328-T1

A 250 mL reaction containing 450 g/L sucrose and B. subtilis crude protein extract (5% v/v) containing a version of GTF5328 from Streptococcus troglodytae Mark (GI: 440355328; Example 14A) having additional C terminal truncations of part of the glucan binding domains (GTF5328-T1, Examples 14A and 14B) in distilled, deionized H₂O, was stirred at pH 5.5 and 47° C. for 4 h, then heated to 90° C. for 30 min to inactivate the enzymes. The resulting product mixture was centrifuged and the resulting supernatant analyzed by HPLC for soluble monosaccharides, disaccharides and oligosaccharides (Table 13), then the oligosaccharides were isolated from the supernatant by SEC at 40° C. using Diaion UBK 530 (Na⁺ form) resin (Mitsubishi). The SEC fractions that contained oligosaccharides ≧DP3 were combined and concentrated by rotary evaporation for analysis by HPLC (Table 13). The combined SEC fractions were diluted to 5 wt % dry solids (DS) and freeze-dried to produce the fiber as a dry solid.

TABLE 13
Soluble oligosaccharide fiber produced by GTF5328-T1.
450 g/L sucrose, GTF5328-T1, 47° C., 4 h

| | Product | SEC-purified | SEC-purified |
|---|---|---|---|
| | mixture, | product, | product |
| | g/L | g/L | % (wt/wt DS) |
| DP8+ | 91.3 | 69.2 | 57.6 |
| DP7 | 21.2 | 14.1 | 11.8 |
| DP6 | 21.2 | 13.3 | 11.1 |
| DP5 | 19.4 | 10.5 | 8.7 |
| DP4 | 14.9 | 6.8 | 5.7 |
| DP3 | 10.9 | 3.7 | 3.1 |
| DP2 | 13.6 | 2.2 | 1.8 |
| Sucrose | 5.3 | 0.0 | 0.0 |
| Leucrose | 94.2 | 0.2 | 0.2 |
| Glucose | 8.4 | 0.0 | 0.0 |
| Fructose | 161.6 | 0.0 | 0.0 |
| Sum DP2-DP8+ | 194.3 | 119.9 | 99.8 |
| Sum DP3-DP8+ | 178.7 | 117.7 | 98.0 |

### Example 14F

### Isolation of Soluble Oligosaccharide Fiber Produced by the C-Terminal Truncated GTF5330-T1

A 250 mL reaction containing 450 g/L sucrose and B. subtilis crude protein extract (5% v/v) containing a version of GTF5330 from Streptococcus mutans UA113 (GI: 440355330; Example 14A) having additional C terminal truncations of part of the glucan binding domains (GTF5330-T1, Examples 14A and 14B) in distilled, deionized H₂O, was stirred at pH 5.5 and 47° C. for 4 h, then heated to 90° C. for 30 min to inactivate the enzymes. The resulting product mixture was centrifuged and the resulting supernatant analyzed by HPLC for soluble monosaccharides, disaccharides and oligosaccharides (Table 14), then the oligosaccharides were isolated from the supernatant by SEC at 40° C. using Diaion UBK 530 (Na⁺ form) resin (Mitsubishi). The SEC fractions that contained oligosaccharides ≧DP3 were combined and concentrated by rotary evaporation for analysis by HPLC (Table 14). The combined SEC fractions were diluted to 5 wt % dry solids (DS) and freeze-dried to produce the fiber as a dry solid.

TABLE 14
Soluble oligosaccharide fiber produced by GTF5330-T1.
450 g/L sucrose, GTF5330-T1, 47° C., 4 h

| | Product | SEC-purified | SEC-purified |
|---|---|---|---|
| | mixture, | product, | product |
| | g/L | g/L | % (wt/wt DS) |
| DP8+ | 89.5 | 67.5 | 56.6 |
| DP7 | 22.1 | 14.3 | 12.0 |
| DP6 | 22.0 | 12.8 | 10.7 |
| DP5 | 19.1 | 10.6 | 8.9 |
| DP4 | 14.3 | 7.0 | 5.9 |
| DP3 | 11.6 | 4.2 | 3.5 |
| DP2 | 15.7 | 2.8 | 2.3 |
| Sucrose | 6.1 | 0.0 | 0.0 |
| Leucrose | 87.0 | 0.2 | 0.2 |
| Glucose | 8.5 | 0.0 | 0.0 |
| Fructose | 162.9 | 0.0 | 0.0 |
| Sum DP2-DP8+ | 194.3 | 119.1 | 99.8 |
| Sum DP3-DP8+ | 178.7 | 116.3 | 97.5 |

### Example 14G

### Isolation of Soluble Oligosaccharide Fiber Produced by the C-Terminal Truncated GTF5330-T3

A 250 mL reaction containing 450 g/L sucrose and B. subtilis crude protein extract (5% v/v) containing a version of GTF5330 from Streptococcus mutans UA113 (GI: 440355330; Example 14A) having additional C terminal truncations of part of the glucan binding domains (GTF5330-T3, Examples 14A and 14B) in distilled, deionized H₂O, was stirred at pH 5.5 and 47° C. for 4 h, then heated to 90° C. for 30 min to inactivate the enzymes. The resulting product mixture was centrifuged and the resulting supernatant analyzed by HPLC for soluble monosaccharides, disaccharides and oligosaccharides (Table 15), then the oligosaccharides were isolated from the supernatant by SEC at 40° C. using Diaion UBK 530 (Na⁺ form) resin (Mitsubishi). The SEC fractions that contained oligosaccharides ≧DP3 were combined and concentrated by rotary evaporation for analysis by HPLC (Table 15). The combined SEC fractions were diluted to 5 wt % dry solids (DS) and freeze-dried to produce the fiber as a dry solid.

TABLE 15
Soluble oligosaccharide fiber produced by GTF5330-T3.
450 g/L sucrose, GTF5330-T3, 47° C., 4 h

| | Product | SEC-purified | SEC-purified |
|---|---|---|---|
| | mixture, | product, | product |
| | g/L | g/L | % (wt/wt DS) |
| DP8+ | 98.0 | 64.7 | 53.7 |
| DP7 | 23.8 | 15.1 | 12.6 |
| DP6 | 22.5 | 13.2 | 11.0 |
| DP5 | 19.4 | 10.5 | 8.8 |
| DP4 | 16.2 | 7.7 | 6.4 |
| DP3 | 15.5 | 4.9 | 4.1 |
| DP2 | 22.4 | 3.5 | 2.9 |
| Sucrose | 6.9 | 0.3 | 0.2 |
| Leucrose | 79.4 | 0.3 | 0.2 |
| Glucose | 9.5 | 0.0 | 0.0 |
| Fructose | 162.2 | 0.0 | 0.0 |
| Sum DP2-DP8+ | 217.8 | 119.8 | 99.5 |
| Sum DP3-DP8+ | 195.4 | 116.2 | 96.6 |

### Example 14H

### Anomeric Linkage Analysis of Soluble Oligosaccharide Fiber Produced by C-Terminal Truncated GTF-0088 Homologs

Solutions of chromatographically-purified soluble oligosaccharide fibers prepared as described in Examples 14C-14G were dried to a constant weight by lyophilization, and the resulting solids analyzed by ¹H NMR spectroscopy and by GC/MS as described in the General Methods section (above). The anomeric linkages for each of these soluble oligosaccharide fiber mixtures are reported in Tables 16 and 17, and compared to the soluble oligosaccharide fiber prepared using the non C-terminal truncated GTF0088 (Example 9).

TABLE 16
Anomeric linkage analysis of soluble oligosaccharides
by ¹H NMR spectroscopy.

| | | % | % | % | % | % | % |
|---|---|---|---|---|---|---|---|
| | | α- | α- | α- | α- | α- | α- |
| Example # | GTF | (1,4) | (1,3) | (1,2) | (1,3,6) | (1,2,6) | (1,6) |
| 9 | GTF0088 | 0.0 | 7.8 | 0.0 | 1.3 | 0 | 90.9 |
| 14C | GTF0088-T1 | 0.0 | 8.0 | 0.0 | 5.2 | 0.0 | 86.8 |
| 14D | GTF5318-T1 | 0.0 | 6.8 | 0.0 | 1.1 | 0.0 | 92.1 |
| 14E | GTF5328-T1 | 0.0 | 8.9 | 0.0 | 1.1 | 0.0 | 90.1 |
| 14F | GTF5330-T1 | 0.0 | 7.5 | 0.0 | 1.1 | 0.0 | 91.4 |
| 14G | GTF5330-T3 | 0.0 | 6.8 | 0.0 | 1.7 | 0.0 | 91.5 |

TABLE 17
Anomeric linkage analysis of soluble oligosaccharides by GC/MS.

| | | | | | | | | | % |
|---|---|---|---|---|---|---|---|---|---|
| | | % | % | % | % | % | % | % | α-(1,4,6) + |
| Example # | GTF | α-(1,4) | α-(1,3) | (1,3,6) | α-(1,2) | α-(1,6) | (1,3,4) | α-(1,2,3) | α-(1,2,6) |
| 9 | GTF0088 | 0.6 | 14.0 | 1.4 | 0.9 | 80.8 | 0.0 | 0.0 | 1.2 |
| 14C | GTF0088-T1 | 1.6 | 20.4 | 2.0 | 0.4 | 74.1 | 0.1 | 0.1 | 1.3 |
| 14D | GTF5318-T1 | 1.7 | 17.0 | 3.6 | 0.5 | 77.2 | 0.0 | 0.1 | 0.0 |
| 14E | GTF5328-T1 | 1.3 | 19.0 | 2.1 | 0.4 | 75.8 | 0.0 | 0.0 | 1.4 |
| 14F | GTF5330-T1 | 1.6 | 14.3 | 2.7 | 0.4 | 79.3 | 0.0 | 0.0 | 1.6 |
| 14G | GTF5330-T3 | 1.7 | 15.0 | 2.0 | 0.4 | 79.7 | 0.2 | 0.1 | 1.0 |

### Example 141

### Viscosity of Soluble Oligosaccharide Fiber

Solutions of chromatographically-purified soluble oligosaccharide fibers prepared as described in Examples 6, 9 and 10 were dried to a constant weight by lyophilization, and the resulting solids were used to prepare a 12 wt % solution of soluble fiber in distilled, deionized water. The viscosity of the soluble fiber solutions (reported in centipoise (cP), where 1 cP=1 millipascal-s (mPa-s)) (Table 18) was measured at 20° C. as described in the General Methods section.

TABLE 18
Viscosity of 12% (w/w) soluble oligosaccharide fiber solutions
measured at 20° C. (ND = not determined).

| Example # | GTF | viscosity (cP) |
|---|---|---|
| 6 | GTF0544/MUT3264 | 6.7 |
| 9 | GTF-C GI:3130088 | 1.8 |
| 10 | GTF GI:387786207 | 1.7 |
| 14D | GTF5318-T1 | 4.1 |
| 14E | GTF5328-T1 | 4.1 |
| 14F | GTF5330-T1 | 4.1 |
| 14G | GTF5330-T3 | 1.7 |

### Example 14J

### Digestibility of Soluble Oligosaccharide Fiber Produced by C-Terminal Truncated GTF-0088 Homologs

Solutions of chromatographically-purified soluble oligosaccharide fibers prepared as described in Examples 14C-14G were dried to a constant weight by lyophilization. The digestibility test protocol was adapted from the Megazyme Integrated Total Dietary Fiber Assay (AOAC method 2009.01, Ireland). The final enzyme concentrations were kept the same as the AOAC method: 50 Unit/mL of pancreatic α-amylase (PAA), 3.4 Units/mL for amyloglucosidase (AMG). The substrate concentration in each reaction was 25 mg/mL as recommended by the AOAC method. The total volume for each reaction was 1 mL. Every sample was analyzed in duplicate with and without the treatment of the two digestive enzymes. The amount of released glucose was quantified by HPLC with the Aminex HPX-87C Columns (BioRad) as described in the General Methods, and compared to the digestibility of the soluble oligosaccharide fiber prepared using the non C-terminal truncated GTF0088 (Example 9) (Table 19).

TABLE 19
Digestibility of soluble oligosaccharide fiber.

| Example # | GTF | Digestibility (%) |
|---|---|---|
| 9 | GTF0088 | 5.6 |
| 14C | GTF0088-T1 | 11.8 |
| 14D | GTF5318-T1 | 6.0 |
| 14E | GTF5328-T1 | 7.6 |
| 14F | GTF5330-T1 | 7.7 |
| 14G | GTF5330-T3 | 3.2 |

### Example 15

### In Vitro Gas Production Using Soluble Oligosaccharide/Polysaccharide Fiber as Carbon Source

Solutions of chromatographically-purified soluble oligosaccharide/polysaccharide fibers were dried to a constant weight by lyophilization. The individual soluble oligosaccharide/polysaccharide soluble fiber samples were subsequently evaluated as carbon source for in vitro gas production using the method described in the General Methods. PROMITOR® 85 (soluble corn fiber, Tate & Lyle), NUTRIOSE® FM06 (soluble corn fiber or dextrin, Roquette), FIBERSOL-2® 600F (digestion-resistant maltodextrin, Archer Daniels Midland Company & Matsutani Chemical), ORAFTI® GR (inulin from Beneo, Mannheim, Germany), LITESSE® Ultra™ (polydextrose, Danisco), GOS (galactooligosaccharide, Clasado Inc., Reading, UK), ORAFTI® P95 (oligofructose (fructooligosaccharide, FOS, Beneo), LACTITOL MC (4-O-β-D-Galactopyranosyl-D-glucitol monohydrate, Danisco) and glucose were included as control carbon sources. Table 20 lists the In vitro gas production by intestinal microbiota at 3 h and 24 h. Table 21 lists the in vitro gas production by intestinal microbiota fed fibers produced using truncated enzymes versus the gas production from the microbiota's ingestion of the control substances at 3, 24.5, and/or 26 hours after ingestion.

TABLE 20
In vitro gas production by intestinal microbiota.

| | mL gas | mL gas |
|---|---|---|
| | formation | formation |
| Sample | in 3 h | in 24 h |
| PROMITOR ® 85 | 2.6 | 8.5 |
| NUTRIOSE ® FM06 | 3.0 | 9.0 |
| FIBERSOL-2 ® 600F | 2.8 | 8.8 |
| ORAFTI ® GR | 3.0 | 7.3 |
| LITESSE ® ULTRA ™ | 2.3 | 5.8 |
| GOS | 2.6 | 5.2 |
| ORAFTI ® P95 | 2.6 | 7.5 |
| LACTITOL ® MC | 2.0 | 4.8 |
| Glucose | 2.4 | 5.2 |
| GTF0544/MUT3264 | 3.2 | 6.2 |
| GTF6207 | 2.5 | 6.3 |
| GTF0088 | 3.7 | 7.2 |

TABLE 21
In vitro gas production by intestinal microbiota.

| | | mL gas | mL gas | mL gas |
|---|---|---|---|---|
| | | formation | formation | formation |
| Example # | Sample | in 3 h | in 24.5 h | in 26 h |
| | ORAFTI ® GR | 4.0 | 8.0 | |
| | LITESSE ® ULTRA ™ | 2.0 | 6.0 | |
| | LACTITOL ® MC | 2.0 | 1.5 | |
| | Glucose | 2.0 | 1.5 | |
| 14C | GTF0088-T1 | 3.0 | | 2.5 |
| 14D | GTF5318-T1 | 2.5 | 3.0 | |
| 14E | GTF5328-T1 | 2.5 | 2.5 | |
| 14F | GTF5330-T1 | 2.5 | 2.0 | |
| 14G | GTF5330-T3 | 4.0 | 2.0 | |

### Example 16

### Colonic Fermentation Modeling and Measurement of Fatty Acids

Colonic fermentation was modeled using a semi-continuous colon simulator as described by Mäkivuokko et al. (Nutri. Cancer (2005) 52(1):94-104); in short; a colon simulator consists of four glass vessels which contain a simulated ileal fluid as described by Macfarlane et al. (Microb. Ecol. (1998) 35(2):180-187). The simulator is inoculated with a fresh human faecal microbiota and fed every third hour with new ileal liquid and part of the contents is transferred from one vessel to the next. The ileal fluid contains one of the described test components at a concentration of 1%. The simulation lasts for 48 h after which the content of the four vessels is harvested for further analysis. The further analysis involves the determination of microbial metabolites such as short chain fatty acids (SCFA); also referred to as volatile fatty acids (VFA) and branched chain fatty acids (BCFA). Analysis was performed as described by Holben et al. (Microb. Ecol. (2002) 44:175-185); in short; simulator content was centrifuged and the supernatant was used for SCFA and BCFA analysis. Pivalic acid (internal standard) and water were mixed with the supernatant and centrifuged. After centrifugation, oxalic acid solution was added to the supernatant and then the mixture was incubated at 4° C., and then centrifuged again. The resulting supernatant was analyzed by gas chromatography using a flame ionization detector and helium as the carrier gas. Comparative data generated from samples of LITESSE® ULTRA™ (polydextrose, Danisco), ORAFTI® P95 (oligofructose; fructooligosaccharide, “FOS”, Beneo), lactitol (Lactitol MC (4-O-β-D-galactopyranosyl-D-glucitol monohydrate, Danisco), and a negative control is also provided. The concentration of acetic, propionic, butyric, isobutyric, valeric, isovaleric, 2-methylbutyric, and lactic acid was determined (Table 22).

TABLE 22
Simulator metabolism and measurement of fatty acid production.

| | | | | | | Short Chain | Branched Chain |
|---|---|---|---|---|---|---|---|
| | | | | | | Fatty Acids | Fatty Acids |
| | Acetic | Propionic | Butyric | Lactic | Valeric | (SCFA) | (BCFA) |
| Sample | (mM) | (mM) | (mM) | (mM) | (mM) | (mM) | (mM) |
| GTF0544/ | 327 | 46 | 100 | 32 | 4 | 509 | 3.9 |
| MUT3264 | | | | | | | |
| GTF6207 | 468 | 62 | 161 | 7 | 3 | 701 | 4.0 |
| GTF0088 | 125 | 10 | 27 | 82 | 1.8 | 245 | 1.8 |
| Control | 83 | 31 | 40 | 3 | 6 | 163 | 7.2 |
| LITESSE ® | 256 | 76 | 84 | 1 | 6 | 423 | 5.3 |
| polydextrose | | | | | | | |
| FOS | 91 | 9 | 8 | 14 | — | 152 | 2.1 |
| Lactitol | 318 | 42 | 94 | 52 | — | 506 | 7.5 |

### Example 17

### Preparation of a Yogurt—Drinkable Smoothie

The following example describes the preparation of a yogurt—drinkable smoothie with the present fibers.

TABLE 23

| Ingredients | wt % |
|---|---|
| Distilled Water | 49.00 |
| Supro XT40 Soy Protein Isolate | 6.50 |
| Fructose | 1.00 |
| Grindsted ASD525, Danisco | 0.30 |
| Apple Juice Concentrate (70 Brix) | 14.79 |
| Strawberry Puree, Single Strength | 4.00 |
| Banana Puree, Single Strength | 6.00 |
| Plain Lowfat Yogurt - Greek Style, Cabot | 9.00 |
| 1% Red 40 Soln | 0.17 |
| Strawberry Flavor (DD-148-459-6) | 0.65 |
| Banana Flavor (#29513) | 0.20 |
| 75/25 Malic/Citric Blend | 0.40 |
| Present Soluble Fiber Sample | 8.00 |
| Total | 100.00 |

| Step | |
|---|---|
| No. | Procedure |
| | Pectin Solution Formation |
| 1 | Heat 50% of the formula water to 160° F. (~71.1° C.). |
| 2 | Disperse the pectin with high shear; mix for 10 minutes. |
| 3 | Add the juice concentrates and yogurt; mix for 5-10 minutes |
| | until the yogurt is dispersed. |
| | Protein Slurry |
| 1 | Into 50% of the batch water at 140° F. (60° C.), add the Supro |
| | XT40 and mix well. |
| 2 | Heat to 170° F. (~76.7° C.) and hold for 15 minutes. |
| 3 | Add the pectin/juice/yogurt slurry to the protein solution; mix |
| | for 5 minutes. |
| 4 | Add the fructose, fiber, flavors and colors; mix for 3 minutes. |
| 5 | Adjust the pH using phosphoric acid to the desired range (pH |
| | range 4.0-4.1). |
| 6 | Ultra High Temperature (UHT) process at 224° F. (~106.7° C.) |
| | for 7 seconds with UHT homogenization after heating at 2500/500 |
| | psig (17.24/3.45 MPa) using the indirect steam (IDS) unit. |
| 7 | Collect bottles and cool in ice bath. |
| 8 | Store product in refrigerated conditions. |

### Example 18

### Preparation of a Fiber Water Formulation

The following example describes the preparation of a fiber water with the present fibers.

TABLE 24

| Ingredient | wt % |
|---|---|
| Water, deionized | 86.41 |
| Pistachio Green #06509 | 0.00 |
| Present Soluble Fiber Sample | 8.00 |
| Sucrose | 5.28 |
| Citric Acid | 0.08 |
| Flavor (M748699M) | 0.20 |
| Vitamin C, ascorbic acid | 0.02 |
| TOTAL | 100.00 |

| Step | |
|---|---|
| No. | Procedure |
| 1 | Add dry ingredients and mix for 15 minutes. |
| 2 | Add remaining dry ingredients; mix for 3 minutes |
| 3 | Adjust pH to 3.0 +/− 0.05 using citric acid as shown in |
| | formulation. |
| 4 | Ultra High Temperature (UHT) processing at 224° F. (~106.7° C.) |
| | for 7 seconds with homogenization at 2500/500 psig (17.24/3.45 |
| | MPa). |
| 5 | Collect bottles and cool in ice bath. |
| 6 | Store product in refrigerated conditions. |

### Example 19

### Preparation of a Spoonable Yogurt Formulation

The following example describes the preparation of a spoonable yogurt with the present fibers.

TABLE 25

| Ingredient | wt % |
|---|---|
| Skim Milk | 84.00 |
| Sugar | 5.00 |
| Yogurt (6051) | 3.00 |
| Cultures (add to pH break point) | |
| Present Soluble Fiber | 8.00 |
| TOTAL | 100.00 |

| Step | |
|---|---|
| No. | Procedure |
| 1 | Add dry ingredients to base milk liquid; mix for 5 min. |
| 2 | Pasteurize at 195° F. (~90.6° C.) for 30 seconds, homogenize |
| | at 2500 psig (~17.24 MPa), and cool to 105-110° F. (~40.6- |
| | 43.3° C.). |
| 3 | Inoculate with culture; mix gently and add to water batch or |
| | hot box at 108° F. (~42.2° C.) until pH reaches 4.5-4.6. |
| | Fruit Prep Procedure |
| 1 | Add water to batch tank, heat to 140° F. (~60° C.). |
| 2 | Pre-blend carbohydrates and stabilizers. Add to batch tank |
| | and mix well. |
| 3 | Add Acid to reduce the pH to the desired range (target pH 3.5-4.0). |
| 4 | Add Flavor. |
| 5 | Cool and refrigerate. |

### Example 20

### Preparation of a Model Snack Bar Formulation

The following example describes the preparation of a model snack bar with the present fibers.

TABLE 26

| Ingredients | wt % |
|---|---|
| Corn Syrup 63 DE | 15.30 |
| Present Fiber solution (70 Brix) | 16.60 |
| Sunflower Oil | 1.00 |
| Coconut Oil | 1.00 |
| Vanilla Flavor | 0.40 |
| Chocolate Chips | 7.55 |
| SUPRO ® Nugget 309 | 22.10 |
| Rolled Oats | 18.00 |
| Arabic Gum | 2.55 |
| Alkalized Cocoa Powder | 1.00 |
| Milk Chocolate Coating Compound | 14.50 |
| TOTAL | 100.00 |

| Step No. | Procedure |
|---|---|
| 1 | Combine corn syrup with liquid fiber solution. Warm syrup in |
| | microwave for 10 seconds. |
| 2 | Combine syrup with oils and liquid flavor in mixing bowl. |
| | Mix for 1 minute at speed 2. |
| 3 | Add all dry ingredient in bowl and mix for 45 seconds at |
| | speed 1. |
| 4 | Scrape and mix for another 30 seconds or till dough is |
| | mixed. |
| 5 | Melt chocolate coating. |
| 6 | Fully coat the bar with chocolate coating. |

### Example 21

### Preparation of a High Fiber Wafer

The following example describes the preparation of a high fiber wafer with the present fibers.

TABLE 27

| Ingredients | wt % |
|---|---|
| Flour, white plain | 38.17 |
| Present fiber | 2.67 |
| Oil, vegetable | 0.84 |
| GRINSTED ® CITREM 2-in-1¹ | 0.61 |
| citric acid ester made from sunflower | |
| or palm oil (emulsifier) | |
| Salt | 0.27 |
| Sodium bicarbonate | 0.11 |
| Water | 57.33 |

¹Danisco.

| Step No. | Procedure |
|---|---|
| 1. | High shear the water, oil and CITREM for 20 seconds. |
| 2. | Add dry ingredients slowly, high shear for 2-4 minutes. |
| 3. | Rest batter for 60 minutes. |
| 4. | Deposit batter onto hot plate set at 200° C. top and bottom, |
| | bake for 1 minute 30 seconds |
| 5. | Allow cooling pack as soon as possible. |

### Example 22

### Preparation of a Soft Chocolate Chip Cookie

The following example describes the preparation of a soft chocolate chip cookie with the present fibers.

TABLE 28

| Ingredients | wt % |
|---|---|
| Stage 1 | |
| Lactitol, C | 16.00 |
| Cake margarine | 17.70 |
| Salt | 0.30 |
| Baking powder | 0.80 |
| Eggs, dried whole | 0.80 |
| Bicarbonate of soda | 0.20 |
| Vanilla flavor | 0.26 |
| Caramel flavor | 0.03 |
| Sucralose powder | 0.01 |
| Stage 2 | |
| Present Fiber Solution (70 brix) | 9.50 |
| water | 4.30 |
| Stage 3 | |
| Flour, pastry | 21.30 |
| Flour, high ratio cake | 13.70 |
| Stage Four | |
| Chocolate chips, 100% lactitol, | 15.10 |
| sugar free | |

| Step No. | Procedure |
|---|---|
| 1. | Cream together stage one, fast speed for 1 minute. |
| 2. | Blend stage two to above, slow speed for 2 minutes. |
| 3. | Add stage three, slow speed for 20 seconds. |
| 4. | Scrape down bowl; add stage four, slow speed for 20 |
| | seconds. |
| 5. | Divide into 30 g pieces, flatten, and place onto silicone lined |
| | baking trays. |
| 6. | Bake at 190° C. for 10 minutes approximately. |

### Example 23

### Preparation of a Reduced Fat Short-Crust Pastry

The following example describes the preparation of a reduced fat short-crust pastry with the present fibers.

TABLE 29

| Ingredients | wt % |
|---|---|
| Flour, plain white | 56.6 |
| Water | 15.1 |
| Margarine | 11.0 |
| Shortening | 11.0 |
| Present fiber | 6.0 |
| Salt | 0.3 |

| Step No. | Procedure |
|---|---|
| 1. | Dry blend the flour, salt and present glucan fiber (dry) |
| 2. | Gently rub in the fat until the mixture resembles fine |
| | breadcrumbs. |
| 3. | Add enough water to make a smooth dough. |

### Example 24

### Preparation of a Low Sugar Cereal Cluster

The following example describes the preparation of a low sugar cereal cluster with one of the present fibers.

TABLE 30

| Ingredients | wt % |
|---|---|
| Syrup Binder | 30.0 |
| Lactitol, MC 50% | |
| Present Fiber Solution (70 brix) 25% | |
| Water 25% | |
| Cereal Mix | 60.0 |
| Rolled Oats 70% | |
| Flaked Oats 10% | |
| Crisp Rice 10% | |
| Rolled Oats 10% | |
| Vegetable oil | 10.0 |

| Step No. | Procedure |
|---|---|
| 1. | Chop the fines. |
| 2. | Weight the cereal mix and add fines. |
| 3. | Add vegetable oil on the cereals and mix well. |
| 4. | Prepare the syrup by dissolving the ingredients. |
| 5. | Allow the syrup to cool down. |
| 6. | Add the desired amount of syrup to the cereal mix. |
| 7. | Blend well to ensure even coating of the cereals. |
| 8. | Spread onto a tray. |
| 9. | Place in a dryer/oven and allow to dry out. |
| 10. | Leave to cool down completely before breaking into clusters. |

### Example 25

### Preparation of a Pectin Jelly

The following example describes the preparation of a pectin jelly with the present fibers.

TABLE 31

| Ingredients | wt % |
|---|---|
| Component A | |
| Xylitol | 4.4 |
| Pectin | 1.3 |
| Component B | |
| Water | 13.75 |
| Sodium citrate | 0.3 |
| Citric Acid, anhydrous | 0.3 |
| Component C | |
| Present Fiber Solution (70 brix) | 58.1 |
| Xylitol | 21.5 |
| Component D | |
| Citric acid | 0.35 |
| Flavor, Color | q.s. |

| Step No. | Procedure |
|---|---|
| 1. | Dry blend the pectin with the xylitol (Component A). |
| 2. | Heat Component B until solution starts to boil. |
| 3. | Add Component A gradually, and then boil until completely |
| | dissolved. |
| 4. | Add Component C gradually to avoid excessive cooling of |
| | the batch. |
| 5. | Boil to 113° C. |
| 6. | Allow to cool to <100° C. and then add colour, flavor and |
| | acid (Component D). Deposit immediately into starch molds. |
| 7. | Leave until firm, then de-starch. |

### Example 26

### Preparation of a Chewy Candy

The following example describes the preparation of a chewy candy with the present fibers.

TABLE 32

| Ingredients | wt % |
|---|---|
| Present glucan fiber | 35 |
| Xylitol | 35 |
| Water | 10 |
| Vegetable fat | 4.0 |
| Glycerol Monostearate (GMS) | 0.5 |
| Lecithin | 0.5 |
| Gelatin 180 bloom (40% solution) | 4.0 |
| Xylitol, CM50 | 10.0 |
| Flavor, color & acid | q.s. |

| Step No. | Procedure |
|---|---|
| 1. | Mix the present glucan fiber, xylitol, water, fat, GMS and |
| | lecithin together and then cook gently to 158° C. |
| 2. | Cool the mass to below 90° C. and then add the gelatin |
| | solution, flavor, color and acid. |
| 3. | Cool further and then add the xylitol CM. Pull the mass |
| | immediately for 5 minutes. |
| 4. | Allow the mass to cool again before processing (cut and |
| | wrap or drop rolling). |

### Example 27

### Preparation of a Coffee—Cherry Ice Cream

The following example describes the preparation of a coffee-cherry ice cream with the present fibers.

TABLE 33

| Ingredients | wt % |
|---|---|
| Fructose, C | 8.00 |
| Present glucan fiber | 10.00 |
| Skimmed milk powder | 9.40 |
| Anhydrous Milk Fat (AMF) | 4.00 |
| CREMODAN ® SE 709 | 0.65 |
| Emulsifier & Stabilizer System¹ | |
| Cherry Flavoring U35814¹ | 0.15 |
| Instant coffee | 0.50 |
| Tri-sodium citrate | 0.20 |
| Water | 67.10 |

¹Danisco.

| Step No. | Procedure |
|---|---|
| 1. | Add the dry ingredients to the water, while agitating |
| | vigorously. |
| 2. | Melt the fat. |
| 3. | Add the fat to the mix at 40° C. |
| 4. | Homogenize at 200 bar/70-75° C. |
| 5. | Pasteurize at 80-85° C./20-40 seconds. |
| 6. | Cool to ageing temperature (5° C.). |
| 7. | Age for minimum 4 hours. |
| 8. | Add flavor to the mix. |
| 9. | Freeze in continuous freezer to desired overrun (100% is |
| | recommended). |
| 10. | Harden and storage at −25° C. |

What is claimed is:

1. A soluble α-glucan fiber composition comprising:
a. 10-30% α-(1,3) glycosidic linkages;
b. 65-87% α-(1,6) glycosidic linkages;
c. less than 5% α-(1,3,6) glycosidic linkages;
d. a weight average molecular weight of less than 5000 Daltons;
e. a viscosity of less than 0.25 Pascal second (Pa·s) at 12 wt % in water at 20° C.;
f. a dextrose equivalence (DE) in the range of 4 to 40; and
g. a digestibility of less than 12% as measured by the Association of Analytical Communities (AOAC) method 2009.01;
h. a solubility of at least 20% (w/w) in pH 7 water at 25° C.; and
i. a polydispersity index of less than 5.

2. A carbohydrate composition comprising: 0.01 to 99 wt % (dry solids basis) of the soluble α-glucan fiber composition of claim 1.

3. A food product comprising the soluble α-glucan fiber composition of claim 1 or the carbohydrate composition of any one of claim 2.

4. A method to produce a soluble α-glucan fiber composition comprising:
a. providing a set of reaction components comprising:
i. sucrose;
ii. at least one polypeptide having glucosyltransferase activity, said polypeptide comprising an amino acid sequence having at least 90% identity to a sequence selected from SEQ ID NOs: 1 and 3;
iii. at least one polypeptide having α-glucanohydrolase activity; and
iv. optionally one or more acceptors;
b. combining the set of reaction components under suitable aqueous reaction conditions whereby a product comprising a soluble α-glucan fiber composition is produced; and
c. optionally isolating the soluble α-glucan fiber composition from the product of step (b).

5. The method of claim 4 wherein the α-glucanohydrolase is an endomutanase.

6. The method of claim 5 wherein the endomutanase comprises an amino acid sequence having at least 90% identity to a sequence selected from SEQ ID NOs: 4, 6, 9, and 11.

7. The method of claim 4 wherein the α-glucanohydrolase is an endodextranase.

8. A method to produce the α-glucan fiber composition of claim 1 comprising:
a. providing a set of reaction components comprising:
i. sucrose;
ii. at least one polypeptide having glucosyltransferase activity, said at least one polypeptide comprising an amino acid sequence having at least 90% identity to a sequence selected from SEQ ID NOs: 13, 16, 17, 19, 28, 30, 32, 34, 36, 38, 40, 42, 44, 46, 48, 50, 52, 54, 56, 58, 60, and 62; and
iii. optionally one or more acceptors;
b. combining the set of reaction components under suitable aqueous reaction conditions to form a single reaction mixture, whereby a product mixture comprising glucose oligomers is formed;
c. optionally isolating the soluble α-glucan fiber composition of claim 1 from the product mixture comprising glucose oligomers; and
d. optionally concentrating the soluble α-glucan fiber composition.

9. The method of claim 4 or 8 wherein combining the set of reaction components under suitable aqueous reaction conditions comprises combining the set of reaction components within a food product.

10. A method to make a blended carbohydrate composition comprising combining the soluble α-glucan fiber composition of claim 1 with: a monosaccharide, a disaccharide, glucose, sucrose, fructose, leucrose, corn syrup, high fructose corn syrup, isomerized sugar, maltose, trehalose, panose, raffinose, cellobiose, isomaltose, honey, maple sugar, a fruit-derived sweetener, sorbitol, maltitol, isomaltitol, lactose, nigerose, kojibiose, xylitol, erythritol, dihydrochalcone, stevioside, α-glycosyl stevioside, acesulfame potassium, alitame, neotame, glycyrrhizin, thaumantin, sucralose, L-aspartyl-L-phenylalanine methyl ester, saccharine, maltodextrin, starch, potato starch, tapioca starch, dextran, soluble corn fiber, a resistant maltodextrin, a branched maltodextrin, inulin, polydextrose, a fructooligosaccharide, a galactooligosaccharide, a xylooligosaccharide, an arabinoxylooligosaccharide, a nigerooligosaccharide, a gentiooligosaccharide, hemicellulose, fructose oligomer syrup, an isomaltooligosaccharide, a filler, an excipient, a binder, or any combination thereof.

11. A method to reduce the glycemic index of a food or beverage comprising incorporating into the food or beverage the soluble α-glucan fiber composition of claim 1.

12. A method of inhibiting the elevation of blood-sugar level, lowering lipids, treating constipation, or altering fatty acid production in a mammal comprising a step of administering the soluble α-glucan fiber composition of claim 1 to the mammal.

13. A cosmetic composition, a pharmaceutical composition, or a low cariogenicity composition comprising the soluble α-glucan fiber composition of claim 1.

14. Use of the soluble α-glucan fiber composition of claim 1 in a food composition suitable for consumption by animals, including humans.

15. A composition comprising 0.01 to 99 wt % (dry solids basis) of the soluble α-glucan fiber composition of claim 1 and: a synbiotic, a peptide, a peptide hydrolysate, a protein, a protein hydrolysate, a soy protein, a dairy protein, an amino acid, a polyol, a polyphenol, a vitamin, a mineral, an herbal, an herbal extract, a fatty acid, a polyunsaturated fatty acid (PUFAs), a phytosteroid, betaine, a carotenoid, a digestive enzyme, a probiotic organism or any combination thereof.
